SEQUENCE LISTING (27 sequences)

SEQ ID NO: 1 - DNA, 2037 bp, Drosophila melanogaster
  CDS (1)..(1860)
atggcagccggtgtcttcaagagttttatgcgcgacttctttgcggtgaaatacgatgagcagcgaaatgatccgcaagcggaacgactggatggcaacggacgactgtatcccaactgctcgtcggatgtgtggctgcgatcctgcgagcgggagatagttgatcccattgagggccatcacagcgggcacattcccaaatggatatgcggtagtctgttgcgcaatggacccggcagctggaaggtgggcgacatgaccttcggccatctgttcgactgctccgccctgctgcaccgatttgccattcggaatggacgcgtcacctaccagaatcgcttcgtggacacggaaacactgcgaaagaatcgctctgcccagcggattgtggtcacggagtttggcacagctgctgttccggatccctgtcactcgatcttcgatagatttgcggccatttttcgaccggatagtggaacggataactcgatgatttccatatatcctttcggggatcagtattacacatttacggagacgccttttatgcatagaataaatccctgcactttggccaccgaagcacgaatctgcaccaccgacttcgtgggcgtggtgaaccacacatcgcatccgcatgttcttcccagtggcactgtctacaacctgggcaccacaatgaccagatctggaccggcatacactatactcagtttcccgcacggcgagcagatgttcgaggatgctcatgtggtggccacactgccgtgccgctggaaactgcatcccggttatatgcacaccttcggcttaacggatcactactttgtgattgtggagcagccgttgtccgtttcgcttacggagtatatcaaagcccagctaggtggacagaatttatcggcgtgtctcaagtggttcgaggatcgaccgacactatttcaccttatagatcgggtttccggcaaactggtgcagacctacgaatcggaagccttcttctacctgcacatcatcaactgctttgaacgggatggccacgtggtggtggacatttgcagctacaggaatcccgagatgatcaactgcatgtatctggaggccattgccaatatgcaaacgaatcccaattatgctaccctctttcgtggacgtcccttgagattcgtcctgcccttgggcacaattcctccggcaagcatcgccaagcggggactggtcaagtccttctcccttgctggactaagtgctccgcaggtttctcgcaccatgaagcactcggtctcgcaatatgcggatataacctacatgcccaccaatggaaagcaagccactgctggagaggaaagccccaagcgagatgccaaacgtggccgctatgaggaggagaatcttgtcaatctggttaccatggagggcagtcaagcggaggcgtttcagggcaccaatggcatccaactgcgtccggaaatgctgtgtgattggggctgtgaaacacctaggatctattatgaacggtatatgggcaagaactaccgatacttctacgcgattagctccgatgtggatgcagtgaatccgggcaccctcatcaaggtggatgtgtggaataagagctgtctaacctggtgcgaggagaatgtctatcccagtgagcccatttttgtgccttcgccggatccgaaatccgaggacgatggcgttatcctggcctccatggtgctgggcggtctcaacgatcgctatgtgggcctaattgtgctatgtgccaaaacgatgaccgagctgggccgttgtgatttccataccaatggacccgtgcccaagtgtctccatggatggtttgcacccaatgccatttagatacggaactccttatatgggaagactacttagcttaggagatagggtaaagcatatgcccagtattacgtttagatttagactagagcatttaatcttagaacttagaattttggattcaagacattcgcaataaactcctgccacttgcgctggaacaaaaaaaaaaaaaaa

SEQ ID NO: 2 - protein, 620 aa, Drosophila melanogaster
MAAGVFKSFMRDFFAVKYDEQRNDPQAERLDGNGRLYPNCSSDVWLRSCEREIVDPIEGHHSGHIPKWICGSLLRNGPGSWKVGDMTFGHLFDCSALLHRFAIRNGRVTYQNRFVDTETLRKNRSAQRIVVTEFGTAAVPDPCHSIFDRFAAIFRPDSGTDNSMISIYPFGDQYYTFTETPFMHRINPCTLATEARICTTDFVGVVNHTSHPHVLPSGTVYNLGTTMTRSGPAYTILSFPHGEQMFEDAHVVATLPCRWKLHPGYMHTFGLTDHYFVIVEQPLSVSLTEYIKAQLGGQNLSACLKWFEDRPTLFHLIDRVSGKLVQTYESEAFFYLHIINCFERDGHVVVDICSYRNPEMINCMYLEAIANMQTNPNYATLFRGRPLRFVLPLGTIPPASIAKRGLVKSFSLAGLSAPQVSRTMKHSVSQYADITYMPTNGKQATAGEESPKRDAKRGRYEEENLVNLVTMEGSQAEAFQGTNGIQLRPEMLCDWGCETPRIYYERYMGKNYRYFYAISSDVDAVNPGTLIKVDVWNKSCLTWCEENVYPSEPIFVPSPDPKSEDDGVILASMVLGGLNDRYVGLIVLCAKTMTELGRCDFHTNGPVPKCLHGWFAPNAI

SEQ ID NO: 3 - DNA, 27 bp, Artificial Sequence
  Description of Artificial Sequence CrtE up primer derived from Erwinia herbicola
gcgtcgaccgcggtctacggttaactg

SEQ ID NO: 4 - DNA, 27 bp, Artificial Sequence
  Description of Artificial Sequence CrtE down primer derived from Erwinia herbicola
ggggtacccttgaacccaaaagggcgg

SEQ ID NO: 5 - DNA, 28 bp, Artificial Sequence
  Description of Artificial Sequence CrtI up primer derived from Erwinia herbicola
gctctagacgtctggcgacggcccgcca

SEQ ID NO: 6 - DNA, 27 bp, Artificial Sequence
  Description of Artificial Sequence crtI down primer derived from Erwinia herbicola
gcgtcgacacctacaggcgatcctgcg

SEQ ID NO: 7 - DNA, 40 bp, Artificial Sequence
  Description of Artificial Sequence oligo(T)-adapter primer
gaccacgcgtatcgatgtcgactttttttttttttttttt

SEQ ID NO: 8 - DNA, 20 bp, Artificial Sequence
  Description of Artificial Sequence specific up-primer derived from EST (Acc. AI063857)
gcagccggtgtcttcaagag

SEQ ID NO: 9 - DNA, 21 bp, Artificial Sequence
  Description of Artificial Sequence anchor primer
gaccacgcgtatcgatgtcga

SEQ ID NO: 10 - DNA, 27 bp, Artificial Sequence
  Description of Artificial Sequence primer Gex-up derived from Drosophila melanogaster
ggaattcgcagccggtgtcttcaagag

SEQ ID NO: 11 - DNA, 26 bp, Artificial Sequence
  Description of Artificial Sequence primer Gex-down derived from Drosophila melanogaster
cctcgaggtagtcttcccatataagg

SEQ ID NO: 12 - DNA, 21 bp, Artificial Sequence
  Description of Artificial Sequence RT-PCR up-primer for  -diox derived from Drosophila melanogaster
ctgcaaacggaccgaccacgt

SEQ ID NO: 13 - DNA, 21 bp, Artificial Sequence
  Description of Artificial Sequence RT-PCR down-primer for  -diox derived from Drosophila melanogaster
gcaaatctatcgaagatcgag

SEQ ID NO: 14 - DNA, 20 bp, Artificial Sequence
  Description of Artificial Sequence RT-PCR up-primer for rp49 derived from ribosomal protein rp49
gacttcatccgccaccagtc

SEQ ID NO: 15 - DNA, 22 bp, Artificial Sequence
  Description of Artificial Sequence RT-PCR down-primer for rp49 derived from ribosomal protein rp49
caccaggaacttcttgaatccg

SEQ ID NO: 16 - DNA, 1855 bp, Mus musculus
  CDS (1)..(1596)
atgttgggaccgaagcaaagcctgccatgcattgccccactgctgaccacggcggaggagactctgagtgctgtctctgctcgggtccgaggacatattcctgaatggcttaatggttatctacttcgagttggacctgggaagtttgaatttgggaaggatagatacaatcattggtttgatggaatggcgttgcttcaccagttccgaatggagaggggcacagtgacatacaagagcaagtttctacagagtgacacatataaggccaacagtgctggaggtagaattgtgatctcagaatttggcacgctggcccttcctgacccatgcaagagcatctttgaacgtttcatgtcaaggtttgagccacctactatgactgacaacaccaacgtcaactttgtgcagtacaaaggtgattactacatgagcacagagactaattttatgaataaggtggacattgagatgctggaaaggacagaaaaggtggactggagcaaattcattgctgtgaatggagccactgcacatcctcattacgacccagatgggacagcatacaacatggggaacagctatgggccaagaggttcttgctataatattattcgtgttcctccaaaaaagaaagagcccggggagacgattcacggagcacaggtgctatgttccattgcctccactgagaaaatgaagccttcttactaccatagctttggaatgacaaaaaactacataatctttgtcgaacagcctgtaaagatgaagctgtggaaaataatcacttctaaaatccggggaaagccctttgctgatgggataagctgggagccccagtataacacgcggtttcatgtggtggataaacacactggacagcttctcccaggaatgtactacagcatgccttttcttacctatcatcaaatcaatgcctttgaggaccagggctgtattgtgattgatctgtgctgccaggatgatgggagaagcctagacctttaccaactacagaatctcaggaaagctggagaggggcttgatcaggtctatgagttaaaggcaaagtctttccctcgaagatttgtcttgcccttagatgttagtgtggatgctgctgaaggaaagaacctcagcccactgtcctattcttcagccagcgctgtgaaacagggtgatggagagatctggtgctctcctgaaaatctacaccacgaagacctggaagaggaaggggggattgaattccctcagatcaactatggccgattcaatggcaaaaagtatagtttcttctatggctgcggttttcgacatttggtgggggattctctgattaaggttgacgtgacgaacaagacactaagggtttggagagaagaaggcttttatccctcggagcccgtttttgttccggtgccaggagcagatgaggaagacagtggggttatactctctgtggtgatcactcccaaccagagtgaaagcaacttcctccttgtcttggatgccaagagcttcacagagctggggcgagcggaagtacccgtgcagatgccttacgggttccatggcacctttgtgcctatctgacggcagaggcgcaaggaaggctaggatcgggcttcgatgagcacactctgaggaaaagagaaaatggtggatctcactcaaaagctgttgtagtttggacctgaccctgacccctaaggaatcatagacccgactcccgtgggctcatcgaccctgacccccaacgtgctgatagatcctgaccaccacgggatcatatttaaattcttgttcccagcttgtggcaatacttttttttttttttgtagcagtggta

SEQ ID NO: 17 - protein, 532 aa, Mus musculus
MLGPKQSLPCIAPLLTTAEETLSAVSARVRGHIPEWLNGYLLRVGPGKFEFGKDRYNHWFDGMALLHQFRMERGTVTYKSKFLQSDTYKANSAGGRIVISEFGTLALPDPCKSIFERFMSRFEPPTMTDNTNVNFVQYKGDYYMSTETNFMNKVDIEMLERTEKVDWSKFIAVNGATAHPHYDPDGTAYNMGNSYGPRGSCYNIIRVPPKKKEPGETIHGAQVLCSIASTEKMKPSYYHSFGMTKNYIIFVEQPVKMKLWKIITSKIRGKPFADGISWEPQYNTRFHVVDKHTGQLLPGMYYSMPFLTYHQINAFEDQGCIVIDLCCQDDGRSLDLYQLQNLRKAGEGLDQVYELKAKSFPRRFVLPLDVSVDAAEGKNLSPLSYSSASAVKQGDGEIWCSPENLHHEDLEEEGGIEFPQINYGRFNGKKYSFFYGCGFRHLVGDSLIKVDVTNKTLRVWREEGFYPSEPVFVPVPGADEEDSGVILSVVITPNQSESNFLLVLDAKSFTELGRAEVPVQMPYGFHGTFVPI

SEQ ID NO: 18 - DNA, 2134 bp, Danio rerio
  CDS (29)..(1675)
aagatagcaatccataacacctaaagtcatgtctacatctgcaaatgatcaaatgtataaagtgccagctaacaaaaaacgtccatctgccagcggcctggagttcatcggtcctcttgtcagctctgttgaggagatcccggatcccatcactacactcattaaaggtcaaattccctcctggatcaacggcagcttccttagaaatggacctggaaaatttgagtttggtgaaagcaaattcacccactggtttgacggtatggctttgatgcatcgtttcaacattaaggatggccaggtgacctacagcagccgatttttgcaaagtgattcttatgtgcagaactcagagaaaaaccgaattgtggtttctgaatttggtaccctggcaacacctgacccatgcaagaacatcttcgcccgcttcttttcacgctttcagatcccaaaaacaactgataatgcaggagtgaactttgttaagtacaagggagatttctacgtaagcacagagaccaacttcatgcgcaaaattgaccctgtgagcctagaaaccaaagaaaaggtggattggtccaaatttattgcagtcagtgcagccacagctcatccacattatgatcgggaaggagcaacttacaacatgggaaactcatatggccgaaaaggcttcttctaccatatactcagagtaccaccaggtgaaaaacaggacgatgatgctgatctgtctggcgctgaaattctttgctcgattcctgctgctgaccccagaaaaccatcatactaccacagttttgtcatgtcagagaattacatagtctttattgagcagccgatcaagctggacctgctgaagttcatgctgtacagaattgctggaaagagctttcataaggtcatgtcctggaacccggaactagacacaatctttcatgtggcagaccgacacacaggccagctcctcaacacaaaatactacagcagtgccatgttcgccctgcaccagattaatgcatatgaagagaatggatatctgattatggacatgtgctgcggagatgatggcaatgtgattggtgaattcacactggagaatctacagtcgaccggggaagatctcgacaagtttttcaattcactgtgtacaaacttaccacgccgatatgtactgcctctggaggtgaaggaggatgaacccaatgaccaaaacctcatcaatttgccatacaccaccgctagcgctgtgaaaactcaaactggggtgttcctctaccatgaggatctctacaatgatgacctgttgcagtacggtggtcttgagtttccacagataaactacgctaactacaacgctcgtccttatcggtatttctatgcctgtggctttggtcatgtgtttggtgactctctgcttaagatggatttggagggaaagaagctgaaggtgtggcgccatgctggtttgttcccctcagaaccagtgtttattccagcacctgatgctcaggatgaggatgatggcgtggtcatgtctgtgatcattacacctagagagaaaaagagcagtttcctacttgtccttgatgccaagacgttcacagagctcggacgagcagaagttccagtggacatcccatacggcactcatggactcttcaatgagaagagctaaacagaaaatctatcattaaaatatctaatcaaacaatttcactcattttgataatttccatctaaacagggaagagttttttgtaatggagtagtgttttttgtattatgcctgattttccttggctgattgtgatttagtattggtacagtatatttgggtgaaggatctgttataatagggcttttacttatgctttttcgaataagttaagcatgatgttaatctattgtatttatatattctctacagcattttttgttattcaagtgcatattttattcatgtatattttatacttacttttatatacattttaatagttttactttttttaaatatacaaattaattacatctgtgaaatttgtgagaccctcgcctgcaaacccagctcagtggattagccatgtaattcttttttaataaatgttgtgccttaaaaaaaaaaaaaaaa

SEQ ID NO: 19 - protein, 549 aa, Danio rerio
MSTSANDQMYKVPANKKRPSASGLEFIGPLVSSVEEIPDPITTLIKGQIPSWINGSFLRNGPGKFEFGESKFTHWFDGMALMHRFNIKDGQVTYSSRFLQSDSYVQNSEKNRIVVSEFGTLATPDPCKNIFARFFSRFQIPKTTDNAGVNFVKYKGDFYVSTETNFMRKIDPVSLETKEKVDWSKFIAVSAATAHPHYDREGATYNMGNSYGRKGFFYHILRVPPGEKQDDDADLSGAEILCSIPAADPRKPSYYHSFVMSENYIVFIEQPIKLDLLKFMLYRIAGKSFHKVMSWNPELDTIFHVADRHTGQLLNTKYYSSAMFALHQINAYEENGYLIMDMCCGDDGNVIGEFTLENLQSTGEDLDKFFNSLCTNLPRRYVLPLEVKEDEPNDQNLINLPYTTASAVKTQTGVFLYHEDLYNDDLLQYGGLEFPQINYANYNARPYRYFYACGFGHVFGDSLLKMDLEGKKLKVWRHAGLFPSEPVFIPAPDAQDEDDGVVMSVIITPREKKSSFLLVLDAKTFTELGRAEVPVDIPYGTHGLFNEKS

SEQ ID NO: 20 - DNA, 1934 bp, Homo sapiens
  CDS (1)..(1668)
atggtgtaccggctcccagttttcaaaaggtacatgggaaatactcctcagaaaaaagccgtctttgggcagtgtcggggtctgccatgtgttgcaccgctgctgaccacagtggaagaggctccacggggcatctctgctcgagtctggggacattttcctaagtggctcaatggctctctacttcgaattggacctgggaaattcgagtttgggaaggataagtacaatcattggtttgatgggatggcgctgcttcaccagttcagaatggcaaagggcacagtgacatacaggagcaagtttctacagagtgatacatataaggccaacagtgctaaaaaccgaattgtgatctcagaatttggcacactggctctcccggatccatgcaagaatgtttttgaacgtttcatgtccaggtttgagctgcctggtaaagctgcagccatgactgacgatactaatgtcaactatgtgcggtacaagggtgattactacctctgcaccgagaccaactttatgaataaagtggacattgaaactctggaaaaaacagaaaaggtagattggagcaaatttattgctgtgaatggagcaactgcacatcctcattatgacccggatggaacagcatacaatatggggaactcctttgggccatatggtttctcctataaggttattcgggttcctccagaggaggtggaccttggggagacaatccatggagtccaggtgatatgttctattgcttctacagagaaagggaaaccttcttactaccatagctttggaatgacaaggaactatataattttcattgaacaacctctaaagatgaacctgtggaaaattgccacttctaaaattcggggaaaggccttttcagatgggataagctgggaaccccagtgtaatacgcggtttcatgtggtggaaaaacgcactggacagctccttccagggagatactacagcaaaccttttgttacatttcatcaaatcaatgcctttgaggaccagggctgtgttataattgatttgtgctgtcaagataatggaagaaccctagaagtttaccagttacagaatctcaggaaggctggggaagggcttgatcaggtccataattcagcagccaaatctttccctcgaaggtttgttttgcctttaaatgtcagtttgaatgcccctgagggagacaacctgagtccattgtcctatacttcagccagtgctgtgaaacaggctgatggaacgatctgctgctctcatgaaaatctacatcaggaggacctagaaaaggaaggaggcattgaatttcctcagatctactatgatcgattcagtggcaaaaagtatcatttcttttatggctgtggctttcggcatttagtgggggattctctgatcaaggttgatgtggtgaataagacactgaaggtttggagagaagatggcttttatccctcagaacctgtttttgttccagcaccaggaaccaatgaagaagatggtggggttattctttctgtggtgatcactcccaaccagaatgaaagcaattttctcctagttttggatgccaagaactttgaagagctgggccgagcagaggtacctgtgcagatgccttatgggttccatggtaccttcatacccatctgatgggacaaccacaaggtctggaaactaggtttaaaataagtgtgcacttggacataaagactggagaaataaacactgaggactccaaaaggggggcaaggaggaagaggggcaggggttaaaaagctacctattgaatactatgttccctatttgggtgatgggttcgttagaagtccaaacctcagcagcacacaatatactcatgtaacaagcctgcacatgtaccccagaatttaaaataaaatttttttttttttttt

SEQ ID NO: 21 - protein, 556 aa, Homo sapiens
MVYRLPVFKRYMGNTPQKKAVFGQCRGLPCVAPLLTTVEEAPRGISARVWGHFPKWLNGSLLRIGPGKFEFGKDKYNHWFDGMALLHQFRMAKGTVTYRSKFLQSDTYKANSAKNRIVISEFGTLALPDPCKNVFERFMSRFELPGKAAAMTDDTNVNYVRYKGDYYLCTETNFMNKVDIETLEKTEKVDWSKFIAVNGATAHPHYDPDGTAYNMGNSFGPYGFSYKVIRVPPEEVDLGETIHGVQVICSIASTEKGKPSYYHSFGMTRNYIIFIEQPLKMNLWKIATSKIRGKAFSDGISWEPQCNTRFHVVEKRTGQLLPGRYYSKPFVTFHQINAFEDQGCVIIDLCCQDNGRTLEVYQLQNLRKAGEGLDQVHNSAAKSFPRRFVLPLNVSLNAPEGDNLSPLSYTSASAVKQADGTICCSHENLHQEDLEKEGGIEFPQIYYDRFSGKKYHFFYGCGFRHLVGDSLIKVDVVNKTLKVWREDGFYPSEPVFVPAPGTNEEDGGVILSVVITPNQNESNFLLVLDAKNFEELGRAEVPVQMPYGFHGTFIPI

SEQ ID NO: 22 - DNA, 21 bp, Artificial Sequence
  Description of Artificial Sequence RT-PCR up-primer for beta-diox I
atggagataatatttggccag

SEQ ID NO: 23 - DNA, 19 bp, Artificial Sequence
  Description of Artificial Sequence RT-PCR down-primer for beta-diox I
aactcagacaccacgattc

SEQ ID NO: 24 - DNA, 21 bp, Artificial Sequence
  Description of Artificial Sequence RT-PCR up-primer for beta-diox II
atgttgggaccgaagcaaagc

SEQ ID NO: 25 - DNA, 21 bp, Artificial Sequence
  Description of Artificial Sequence RT-PCR down-primer for beta-diox II
tgtgctcatgtagtaatcacc

SEQ ID NO: 26 - DNA, 21 bp, Artificial Sequence
  Description of Artificial Sequence RT-PCR up-primer for beta-actin
ccaaccgtgaaaagatgaccc

SEQ ID NO: 27 - DNA, 21 bp, Artificial Sequence
  Description of Artificial Sequence RT-PCR down-primer for beta-actin
cagcaatgcctgggtacatgg

(19) United States
(12) Patent Application Publication
(10) Pub. No.: US 2003/0166595 A1
(43) Pub. Date: Sep. 4, 2003

(54) Novel deoxygenases catalyzing cleavage of beta-carotene

(76) Inventors: Johannes Von Lintig, Freiburg im Breisgau (DE); Klaus Vogt, Frelburg im Brelsgau (DE)

Correspondence Address: SYNGENTA BIOTECHNOLOGY, INC., PATENT DEPARTMENT, 3054 CORNWALLIS ROAD, P.O. BOX 12257, RESEARCH TRIANGLE PARK, NC, 27709-2257, US

(21) Appl. No.: 10/168,517

(22) PCT Filed: Dec. 27, 2000
(86) PCT No.: PCT/EP00/13273

(30) Foreign Application Priority Data
Mar. 20, 2000 (EP) 00105822.1

(51) Int. Cl. A61K 48/00; C12Q 1/68; G01N 33/53; C12P 21/02; A61K 39/395; C12N 9/02; A01H 1/00; C12N 15/82; C12N 5/04

(52) U.S. Cl. 514/44; 435/189; 435/69.1; 435/320.1; 435/419; 800/282; 530/388.26; 424/146.1; 435/6; 435/7.1

(57) ABSTRACT

The present invention provides means and methods of transforming bacteria, fungi including yeast, animal and plant cells, seeds, tissues and whole plants in order to yield transformants capable of expressing novel β-carotene dioxygenases and accumulating important metabolites of the carotene/retinoid pathway such as vitamin A aldehyde and retinoic acid. The present invention further provides means and methods to biotechnically produce retinoids using cells, tissues, organs or whole organisms which natively or after transformation accumulate β-carotene or which take up β-carotene from the medium. The present invention also provides DNA molecules encoding symmetrically and asymmetrically cleaving β-carotene dioxygenases derived from different sources and taxonomic groups of living organisms designed to be suitable for carrying out the method of the invention, and plasmids or vector systems comprising said molecules. Furthermore, the present invention provides transgenic bacteria, fungi including yeast, animal and plant cells, seeds, tissues and whole plants that display an improved nutritional quality or physiological condition and contain such DNA molecules and/or that have been generated by use of the methods of the present invention.

The present invention relates to the field of transformation of bacteria, yeast, fungi, insect, animal and plant cells, seeds, tissues and whole organisms. More specifically, the present invention relates to the integration of recombinant nucleic acid sequences coding for one or more specific enzymes of the carotenoid/retinoid biosynthetic pathway into suitable host cells or organisms, which, upon transformation, display a desired phenotype and can be used e.g. for commercial production. Furthermore, the present invention provides diagnostic and therapeutic means designed to address specific features involved in the carotenoid/retinoid pathway. In particular, the present invention provides means and processes to biotechnically achieve oxidative cleavage of C₄₀ carotenoids leading to different metabolites characteristic to the carotenoid/retinoid pathway.

### BACKGROUND OF THE INVENTION

Vitamin A (retinol) and its derivatives (retinal, retinoic acid), for which the term “retinoids” is used throughout the specification, represent a group of chemical compounds involved in a broad range of fundamental physiological processes in animals. They are essential e.g. in vision, reproduction, metabolism, cell differentiation, bone development and pattern formation during embryogenesis. To study the effects of retinoids such as vitamin A several species have been used e.g. mice, rats, chicken and pigs as vertebrate model organisms, while in invertebrates most investigations have been performed with the fruit fly Drosophila melanogaster. The fly visual system has served for decades as a model for receptor multiplicity and vitamin A utilisation using electrophysiology, photochemistry, genetics and molecular biology.

Vitamin A and its most important derivatives retinal and retinoic acid (RA) consist of 20 carbon atoms (C₂₀) and belong to the chemical class of isoprenoids. Animals are, in general, unable to synthesize retinoids de novo. For retinoid biosynthesis animals depend on the uptake of carotenoids with provitamin A activity from their diet. In those animals which are able to synthesize retinoids from carotenoids, the provitamin has to be cleaved enzymatically. In mammals, for example, this enzymatic activity has been described in crude extracts derived from small intestine and from liver. This enzyme catalyses the symmetric oxidative cleavage of β-carotene to form two molecules of retinal and has been characterised biochemically as 15,15′-β-carotene dioxygenase (β-diox I). Such enzymes are involved in carotenoid metabolism/retinoid formation all over the animal kingdom. As an example, the biosynthetic pathway of retinoid formation described in mammals is illustrated in FIGS. 1 and 9. Besides β-carotene, xanthophylls (carotenoids containing oxygen) can also be cleaved as long as they have a non-substituted β-ionone ring (e.g. β-cryptoxanthin), and in different animal species the ability to metabolise carotenoids different from β-carotene to form hydroxylated retinoids has been reported. (e.g. zeaxanthin and lutein in the class of Insecta). For further metabolism the retinal produced has to be enzymatically modified to form retinol (vitamin A) or retinoic acids.

Enzymatic oxidative cleavage of carotenoids is also found in bacteria and plants. In higher plants, many examples for eccentric cleavage of carotenoids are found. These examples include the formation of saffron in crocus, citraurin and other apocarotenoids in citrus fruits, and, most interestingly, the plant hormone abscisic acid (ABA), a growth regulator involved e.g. in the autumnal fall of leaves and in seed dormancy. ABA derives from the oxidative cleavage of 9-cis-epoxy-carotenoids at the 11-12 carbon double bound. Recently, analysis of a maize mutant, vp14, which is defective in ABA biosynthesis, has provided a better molecular understanding of this cleavage reaction and led to the cloning and molecular characterisation of the first carotenoid cleaving enzyme (β-diox I) from animal sources. From this finding arose the question as to how similar enzymes are involved in animal carotenoid/retinoid metabolism catalysing the oxidative cleavage of carotenoids with provitamin A activity. In subsequent experiments, similar enzymes (β-diox II) could indeed be identified and characterized which are also involved in the carotenoid/retinoid pathway and specifically cleave β-carotene to form β-apocarotenal, a precursor of retinoic acid. Thus, besides β-diox I as a novel type of β-carotene specific enzymes, still another novel type of enzymes (β-diox II) could be identified according to the present invention also effecting oxidative cleavage of the same substrate, β-carotene. p In animals, the function of these important types of enzymes for carotenoid metabolism/retinoid formation has been under investigation in vitro for almost 40 years. However, all attempts to isolate and purify the proteins and characterise their molecular structure failed. The disclosure of the molecular structure of these enzymes including their nucleotide sequences (cDNA) and their amino acid sequences would be of importance for the whole variety of fields dealing with vitamin A/retinoid effects in animals and also in medicine. Furthermore, this genetic material can then be used to transform whole living organisms to produce retinoids such as vitamin A and retinoic acid in e.g. plants and microorganisms to enhance their nutritional value.

In vertebrates, symmetric versus asymmetric cleavage of β-carotene in the biosynthesis of vitamin A and its derivatives has been controversially discussed. In addition to β-diox I the present invention provides the identification of cDNAs from mouse, human and zebrafish encoding a second type of carotene dioxygenase termed β-diox II catalyzing exclusively the asymmetric oxidative cleavage of β-carotene resulting in the formation of β-apocarotenal and β-ionone, a substance known as a floral scent from, e.g., roses. Besides β-carotene, lycopene is also oxidatively cleaved by the enzyme. The deduced amino acid sequence shares significant sequence identity with the β,β-carotene-15,15′-dioxygenases and the two enzyme types β-diox I and β-diox II have several conserved motifs. As regards their function, the apo-carotenals formed by this enzyme serve—amongst other possible physiological effects—as precursors for the biosynthesis of retinoic acid. Thus, in contrast to Drosophila, in vertebrates both symmetric and asymmetric cleavage pathways exist for carotenes, revealing a greater complexity of carotene metabolism here.

In humans, as is generally known, retinal, the cleavage product of β-diox I, is a decisive factor in vision. It is similarly clear that enzymes that determine the availability of direct precursors of retinoic acid in the whole organism or within a single cell will have a broad impact on retinoic acid signalling pathways and on cellular responses mediated thereby.

There are several medical applications for retinoids, e.g. in cancer treatment. As active ingredient in a (prophylactic or therapeutic) pharmaceutical preparation, retinoids can serve for the prevention and/or for the treatment of different types of cancer. For instance, animal models have shown that retinoids modulate cell growth, differentiation and apoptosis, and suppress carcinogenesis in several tissues such as e.g. lung, skin, mammary glands, prostate and bladder. The latter also applies to clinical studies with patients displaying premalignant or malignant lesions of the oral cavity, cervix, bronchial ephithelium, skin and other tissues and organs Some retinoids show antitumor activity even with respect to highly malignant cells in vitro, as could be demonstrated by inhibition of proliferation and by induction of differentiation or apoptosis. An outstanding example for a therapeutic effect is the differentiation of promyelocytic leukemia cells to granulocytes caused by all-trans retinoic acid which currently is used successfully in the therapy of this type of cancer [Nason-Burchenal and Dmitrovsky, in: Retinoids, p. 301 (1999); Xu and Lotan, in: Retinoids, p. 323 (1999)].

The present invention provides for the first time a complete molecular characterization of enzymes involved in animal carotenoid/retinoid metabolism catalysing the oxidative cleavage of carotenoids with provitamin A activity. The accomplishment of the present invention including the discovery of complete nucleotide sequences encoding these gene types e.g. permits the improvement of the nutritional status, especially in non-developed countries by providing plants or parts thereof transformed according to the present invention. According to the present invention there is provided a novel type of enzymes termed β-diox II also effecting oxidative cleavage of β-carotene but, in contrast to β-diox I, yielding β-apocarotenal which is the second known precursor of retinoic acid. Therefore, the present invention provides two novel types of enzymes being specific for oxidatively cleaving β-carotene and accumulating precursors of retinoic acid.

For instance, vitamin A deficiency represents a very serious health problem leading to severe clinical symptoms in the part of the world's population living on grains such as rice as the major or almost only staple food. In southeast Asia alone, it is estimated that 5 million children develop the eye disease xerophthalmia every year, of which 0.25 million eventually go blind. Furthermore, although vitamin A deficiency is not a proximal determinant of death, it is correlated with an increased susceptibility to potential fatal afflictions such as diarrhoea, respiratory diseases and childhood diseases, such as measles. According to statistics compiled by UNICEF, improved provitamin nutrition could prevent 1-2 million deaths annually among children aged 1-4 years, and an additional 0.25-0.5 million deaths during later childhood. For these reasons it is very desirable to raise the vitamin A level in staple foods.

In developed countries vitamin deficiency can no longer be regarded as posing a general problem, because sufficient provitamin A is provided by plant food and vitamin A is directly available from animal products. However, for prophylactic reasons or in the context of certain clinical and/or genetic disorders or malfunctions afflicting e.g. resorption or the ability to correctly cleave provitamins to vitamin A, it may be desired to provide retinoids e.g. as functional ingredients of so-called “functional food”.

Despite numerous publications and patents concerning the total chemical synthesis of retinol and its analogs, there is a strong need for the biotechnical production of these substances, which are highly valuable for nutritional, diagnostic and pharmaceutical/therapeutical applications.

### SUMMARY OF THE INVENTION

The present invention provides means and methods of transforming bacteria, yeast, fungi, insect, animal and plant cells, seeds, tissues and whole organisms in order to yield transformants capable of expressing an asymmetrically cleaving β-carotene dioxygenase (β-diox II) polypeptide or functional fragment thereof and accumulating β-apocarotenal and β-ionone as well as apolycopenals. The present invention further provides means and methods to biotechnically produce retinoids using cells, tissues, organs or whole organisms which natively or after transformation accumulate β-carotene or which take up β-carotene from the-medium. The present invention also provides DNA molecules encoding said novel β-carotene dioxygenase derived from different sources and taxonomic groups of living organisms designed to be suitable for carrying out the method of the invention, and plasmids or vector systems comprising said molecules. Furthermore, the present invention provides transgenic bacteria, yeast, fungi, insect, animal and plant cells, seeds, tissues and whole organisms that display an improved nutritional quality or physiological condition and contain the above DNA molecule(s) and/or that have been generated by use of the methods of the present invention. Additionally, the present invention provides antibodies displaying a specific immunoreactivity with a β-diox II polypeptide which are suitable for diagnostic, therapeutic and screening purposes as well as for isolating and purifying said polypeptide. Finally, the present invention provides means and methods for use of the DNA molecules according to the invention in the field of gene therapy.

Thus, the present invention provides both the de novo introduction and expression of the enzyme which cleaves β-carotene in organisms which per se are retinoid-free such as plant material, fungi and bacteria, and the modification of pre-existing retinoid biosynthesis in order to regulate accumulation of certain retinoids of interest. Furthermore, the present invention provides DNA probes and sequence information which allow the person skilled in the art to clone the corresponding genes and/or cDNAs from other sources such as animal species not disclosed throughout the present specification.

Additionally, the present invention provides pharmaceutical preparations comprising the gene products or functional active fragments thereof as active ingredient as well as a simple and suitable diagnostic test system to further prove functionality of these molecules.

### BRIEF DESCRIPTION OF THE DRAWINGS

FIG. 1 shows the main steps in retinoid formation of animals. The key step in vitamin A formation is emphasized with the boldarrow; only the all-trans isomers of the retinoids are shown.

FIG. 2 shows the color shift from yellow (β-carotene) to almost white (retinoids) in β-carotene producing and accumulating E. coli caused by the expression of the β-carotene dioxygenase from D. melanogaster (E. coli⁽⁺⁾ strain) compared to the control (E. coli⁽⁻⁾ strain).

FIG. 3 gives HPLC analyses and spectral characterization, of the retinoids formed in the β-carotene producing E. coli transformed with the plasmid for the expression of the β-carotene dioxygenase cDNA from Drosophila (E. coli⁽⁺⁾-strain) compared to the E. coli⁽⁻⁾-strain transformed with the vector control (pBAD-TOPO). The scale bars indicate an absorbance of 0.01 at 360 nm. A. Formaldehyde/chloroform extracts from E. coli⁽⁺⁾ (upper trace) and E. coli⁽⁻⁾-strain (lower trace). B. Hydroxylamine/methanol extracts yielding the corresponding oximes (syn and anti) from the respective retinal isomers. In the upper trace authentic standards are separated. In the middle trace the isomeric composition of the extracts from the E.coli⁽⁺⁾-strain and in the lower trace the HPLC profile of the extracts from E. coli⁽⁻⁾-strain are shown.

FIG. 4 illustrates the absorbance spectra (in n-hexane) of the main substances extracted from the E. coli⁽⁺⁾-strain compared to those of authentic standards (dotted).

FIG. 5 displays the enzymatic activity of the β-diox-gex fusion protein under different conditions. The fusion protein β-diox-gex was incubated under different conditions in buffer containing 50 mM tricine/NaOH (pH 7.6) and 100 mM NaCl. To start the reaction 5 μl β-carotene (80 μM) disolved in ethanol was added. After 2 h at 30° C. the reactions were stopped and extracted. HPLC-analyses were performed and the HPLC-profiles at 360 nm are shown. The scale bar indicates an absorbance of 0.005 at 360 nm. A.: incubation in the presence of 5 μM FeSO₄ and 10 mM L-ascorbate; B.: Incubation without FeSO₄/ascorbate; C.: Incubation in the presence of 10 mM EDTA; D.: Prior to the incubation the fusion protein was heated for 10 min at 95° C.

FIG. 6 depicts the cDNA sequence and deduced amono acid sequence of β-diox from D. melanogaster.

FIG. 7 is a linear alignment of the deduced amino acid sequences of vp14 (maize), RPE65 (retinal pigment epithelium, bovine) and β-diox I (fruit fly). Identity is indicated by black and conserved amino acids according to the PAM250 matrix are indicated by gray. We used visual alignment and the program Map. A highly conserved region can e.g. be found between position 549 and 570 of the β-diox I sequence. All homologues of β-diox identified so far share this common motif which—amongst others—is characteristic for the enzymes according to the invention.

FIG. 8 illustrates mRNA-levels of β-diox I in diffrent parts of the body. The expression pattern of β-diox mRNA was investigated by RT-PCR. β-diox mRNA was only detectable in the head. The cDNAs were synthesized from total RNA preparations derived from the head, thorax and abdomen of adult Drosophila (females and males). As a control the mRNA levels of the ribosomal protein rp49 (FLYBASE accession number FBgn0002626) was investigated in the same RNA samples using a set of intron-spanning primers.

FIG. 9 is a schematic overview of the mammalian β-carotene/retinoid metabolism. Solid arrows indicate vitamin A formation by the symmetric cleavage pathway. The retinal formed can be further metabolized to give retinol and retinylesters (storage) or can be oxidized to give retinoic acid. Broken arrows indicate β-(8′, 10′, 12′)-apocarotenal formation by the asymmetric cleavage of β-carotene. For retinoic acid formation the β-apocarotenals have to be shortened by a mechanism similar to β-oxidation of fatty acid.

FIG. 10 is a comparison of the deduced amino acid sequences of the two types of carotene dioxygenases in mouse. Linear alignment of the deduced amino acid sequences of the mouse β-diox I (mouse-1) and β-diox II from mouse (mouse-2). Identity is indicated in black, and conserved amino acids, according to the PAM250 matrix, are indicated in gray. Six conserved histidin residues probably involved in binding the cofactor Fe²⁺ are marked by asterisks.

FIG. 11 shows analyses of the products formed in in vitro tests for enzymatic activity conducted with β-diox II. Crude extracts from E. coli expressing β-diox II were incubated in the presence of β-carotene for 2 h. Then, the compounds formed were extracted and HPLC analyses were carried out. A, formaldehyde/chloroform extract; B, hydroxylamine/methanol extract. After extraction in the presence of formaldehyde/chloroform, a compound with a retention of 4.6 min could be detected, while in the presence of hydroxylamine/chloroform its retention time shifted to 16 min. C, UV/VIS spectrum of peak 1. D, UV/VIS spectrum of peak 2.

FIG. 12 shows the colors of β-carotene and lycopene synthesizing and accumulating E. coli strains after expressing either the β-diox I or β-diox II, respectively. A, β-carotene accumulating E. coli control strain; B, β-carotene accumulating strain expressing β-diox; C, β-carotene accumulating strain expressing β-diox II; D,. lycopene accumulating strain expressing β-diox II; E, lycopene accumulating control strain.

FIG. 13 shows the detection of the carotene cleavage products by HPLC analyses of E. coli extracts. HPLC analyses of the carotene cleavage products formed in the β-carotene producing E. coli strain. Bacteria were extracted with the hydroxylamine/methanol method (von Lintig J., and Vogt, K. (2000) J. Biol. Chem. 275, 11915-11920). A, Extract of the E. coli strain expressing β-diox I (upper trace) compared with a control strain (lower trace). The composition of the retinoids found is indicated in the figure. B, Extract of the E. coli strain expressing β-diox II (upper trace) compared with a control strain (lower trace). Six substances could be detected and assigned to two different classes of compounds (class 1: peak 2, 5 and 6; class 2: peak 1, 3, 4) due to their UV/VIS spectra. C, UV/VIS spectrum of peak 2 as a representative of class 1 compounds; D, UV-VIS spectrum of peak 4 as a representative of class 2.

FIG. 14 is a linear alignment of the deduced amino acid sequences of drosophila (fruit fly β-diox I, SEQ ID No. 2), mouse-2 (Mus musculus, SEQ ID No. 17), human-2 (Homo sapiens, SEQ ID No. 21), and zebra-2 (Danio rerio, SEQ ID No. 19). Identity is indicated by black. Arrows indicate regions of postulated homologies to β-diox from drosophila. A highly conserved region can e.g. be found between position 549 and 570 of the β-diox sequence. All homologues of β-diox identified so far share this common motif which is characteristic for the enzymes according to the invention.

FIG. 15 is a phylogenetic tree calculation of the metazoan polyene chain dioxygenases and the plant VP14. Phylogenetic tree calculation was based on a sequence distance method and utilizes the Neighbor Joining (NJ) algorithm (Saito, N., and Nei, M., (1987) Mol. Biol. Evol. 4, 406-425) with the deduced amino acid sequences of all metazoan polyene chain dioxygenases and the plant VP14. The two different types of vertebrate carotene dioxygenases are indicated by the numbers 1 and 2 after the organism's name. Besides the sequences reported here, the following sequences were used human-1 (AAG15380), mouse-1 (Redmond, T. M., Gentleman, S., Duncan, T., Yu, S., Wiggert, B., Gantt, E., and Cunningham, F. X. Jr. (2000) J. Biol. Chem. online), RPE65 human (XP001366), RPE65 bovine (A47143), Drosophila (von Lintig, J., and Vogt, K. (2000) J. Biol. Chem. 275, 11915-11920), VP14 (AAB62181).

FIG. 16 displays an estimation of the steady-state mRNA levels of the two types of carotene dioxygenases in different tissues of mouse. Analyses of β-diox I, β-diox II, and β-actin mRNA levels in various tissues of mouse by RT-PCR analyses. For analyses the reaction products were loaded on a TBE-agarose (1.2%) gel. The gel was stained with ethidium bromide and the photographs are shown. For each sample the analysis was carried out in the presence (+) and in absence of reverse transcriptase (−) demonstrating that PCR products derived from mRNA.

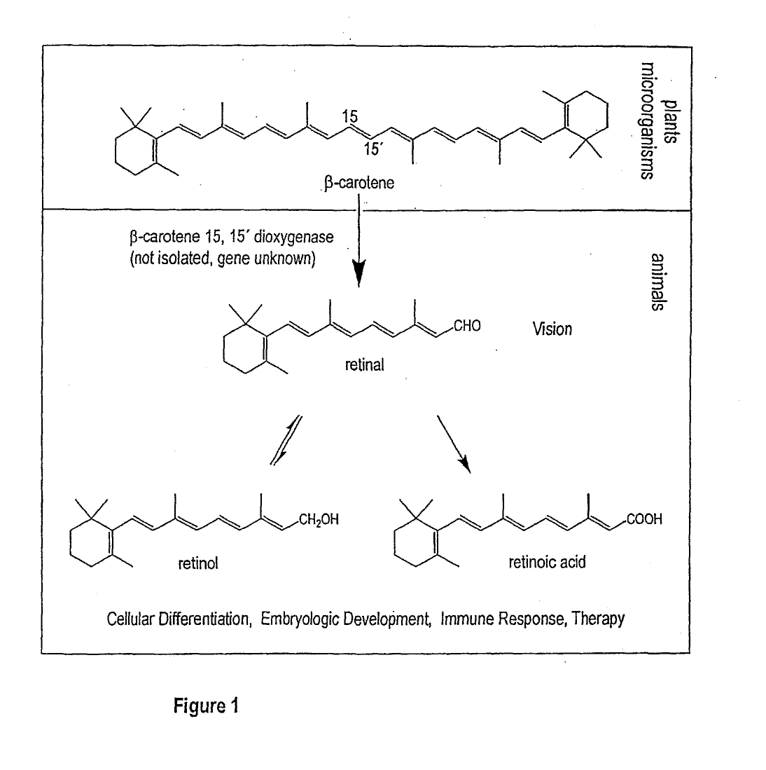

### DETAILED DESCRIPTION OF THE INVENTION

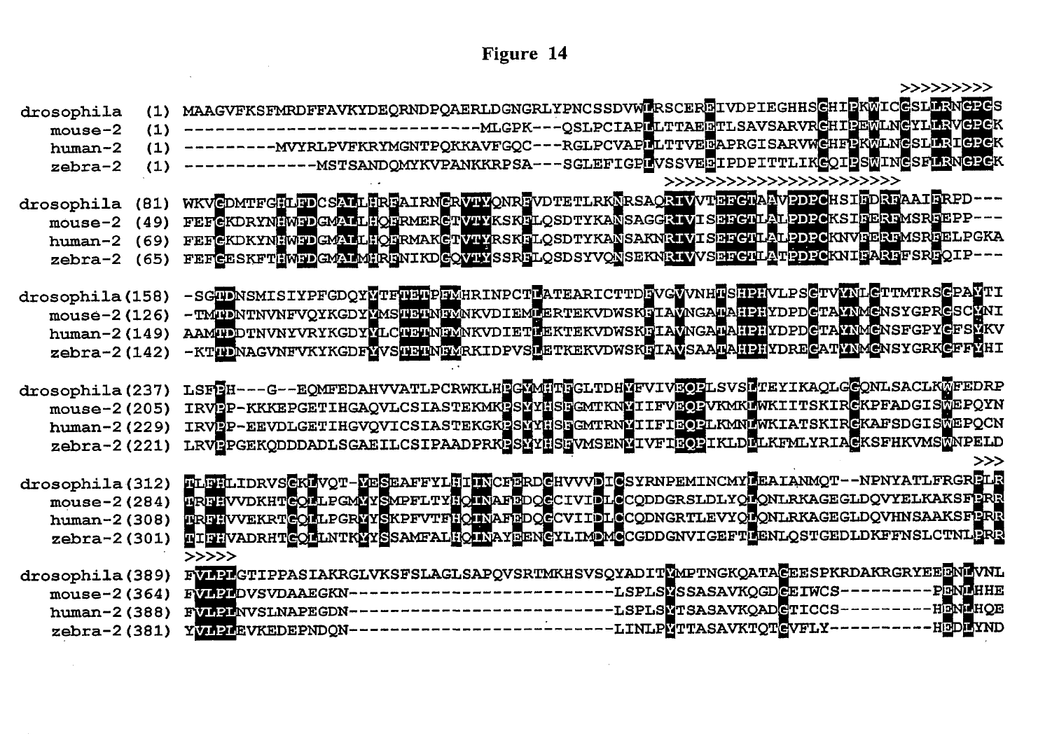

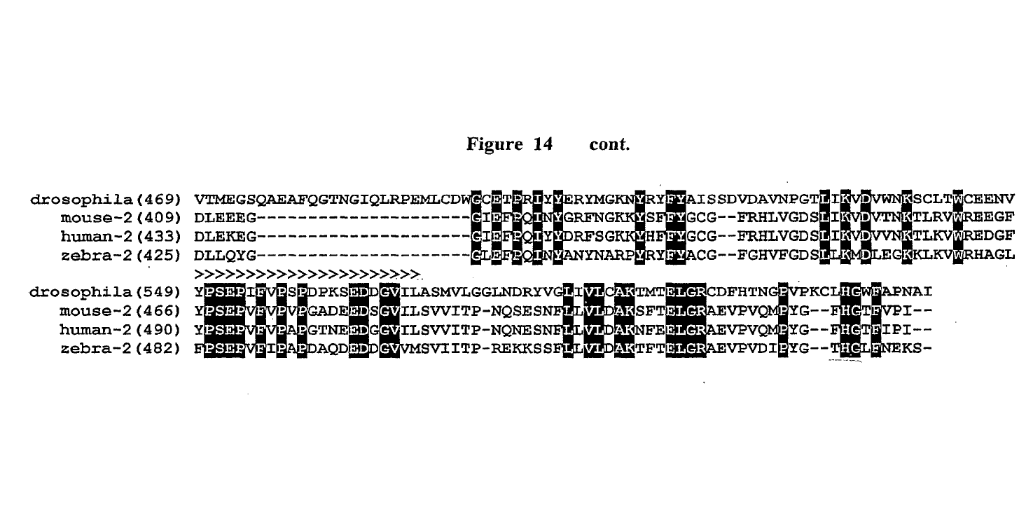

The present invention provides isolated novel β-carotene dioxygenase (β-diox II) polypeptides or functional fragments thereof having the biological activity of specifically cleaving β-carotene and lycopene to form β-apocarotenal and β-ionone, and apolycopenals, respectively. According to a preferred embodiment on the basis of sequence information obtained from mouse, said β-diox II polypeptides or functional fragments thereof comprise e.g. one or more of the amino acid sequences selected from the group consisting of amino acid sequences extending from 29 to 47, 96 to 118, 361 to 368, and 466 to 487 of SEQ ID No. 17, with the second and fourth being preferred. These regions, and in particular the region as set out from position 96 to 118 and from position 466 to position 487 of SEQ ID No. 17, are of particular interest, since they have proven to be highly conserved in nature. Therefore, respective nucleic acid probes derived from the DNA sequence as set out in SEQ ID No. 16 and comprising one or more of the nucleic acid sequences selected from the group consisting of nucleic acid sequences extending from 115 to 141, 286 to 354, 1081 to 1104, and 1396 to 1461 of SEQ ID No. 16, with the second and fourth being preferred, can easily be designed, generated and used by a person skilled in the art as suitable screening tools for expression analysis or to reveal further members of this new type of enzymes having the enzymatic activity as outlined above and are thus encompassed by the present invention. Evidently, as can be taken from FIG. 14, the same applies to homologous β-diox II sequences provided herein. For example, said β-diox II polypeptides or functional fragments thereof comprise e.g. one or more of the amino acid sequences extending from 55 to 63, 112 to 134, 378 to 385, and 482 to 503 of SEQ ID No. 19 (zebrafish), and from 59 to 67, 116 to 138, 385 to 392, and 490 to 511 of SEQ ID No. 21 (human), with the respective second and fourth regions being preferred. Accordingly, respective nucleic acid probes derived from the DNA sequences as set out in SEQ ID Nos. 18 and/or 20 and comprising one or more of the nucleic acid sequences selected from the group consisting of nucleic acid sequences extending from 191 to 217, 362 to 430, 378 to 385, and 482 to 503 of SEQ ID No. 18, and from 175 to 201, 346 to 414, 1153 to 1176, and 1468 to 1533 of SEQ ID No. 20, with the respective second and fourth regions being preferred, can easily be designed, generated and used as already outlined above. All these β-diox II homologues as well as others from still different sources can easily be identified and used according to the principles of the present invention.

The present invention is in part based on the fact that essentially all plants, fungi and bacteria per se are retinoid-free. Although all plants, some fungi and many bacteria are able to synthesize β-carotene, they usually do not have enzymes which enable them to cleave β-carotene to retinoids. These organisms can thus be used according to the invention as source for β-carotene in order to synthesize retinoids after introduction of a e.g. cDNA encoding a β-carotene dioxygenase type II. Furthermore, such organisms which accumulate geranyl-geranyl-diphosphate (GGPP) but natively or otherwise lack downstream enzymes so that essentially no β-carotene is produced, can also be used in the context of the present invention. The synthesis of β-carotene requires the enzyme phytoene synthase (psy) involved in the first carotenoid-specific reaction which comprises a two-step reaction resulting in a head-to head condensation of two molecules of GGPP to form the first, yet uncoloured carotene product, phytoene. Furthermore, the further enzymatic pathway necessitates complementation with three additional plant enzymes: phytoene desaturase (PDS) and ζ-carotene desaturase (ZDS), each catalyzing the introduction of two double bonds, and lycopene β-cyclase. To reduce the transformation effort, a bacterial carotene desaturase such as e.g. CrtI derived from Erwinia, capable of introducing all four double bonds required for the entire desaturation sequence and converting phytoene to lycopene directly, can be used in a preferred embodiment of the present invention [see Xudong Ye et al., “Engineering the Provitamin A (β-Carotene) Biosynthetic Pathway into (Carotenoid-Free) Rice Endosperm”, Science Vol. 287, p. 303-305 (2000)]. For example, a vector capable of preferably expressing both plant phytoene synthase (psy) (GenBank® accession number X78814) and bacterial phytoene desaturase (crtI) (GenBank® accession number D90087) can be used to direct the formation of lycopene in e.g. plastids which normally are essentially carotenoid-free. In addition, a second vector capable of expressing lycopene β-cyclase (GenBank® accession number X98796) can easily be designed and used for co-transformation. However, as could be shown in transformation experiments, it may not be essential to introduce a nucleic acid sequence encoding said lycopene β-cyclase since transformants generated with a single transformation using a combined expression cassette harbouring psy and crtI have shown to accumulate β-carotene as well as lutein and zeaxanthin. To complete the pathway down to formation of retinoids such as retinoic acid or vitamin A and its derivatives, a nucleic acid sequence encoding a polypeptide or functional fragment according to the invention can be introduced either alone or in combination with any of the other enzymes mentioned above. Thus, the present invention enables to completely introduce or complement the carotenoid/retinoid pathway in a given host appropriately selected according to the present invention.

The term “carotenoid-free” or “essentially carotenoid-free” used throughout the specification to differentiate between certain target cells or tissues shall mean that the respective plant or other material not transformed according to the invention is known normally to be essentially free of carotenoids, as is the case for e.g. storage organs such as, for example, rice endosperm and the like. Carotenoid-free does not mean that those cells or tissues that accumulate carotenoids in almost undetectable amounts are excluded. Preferably, said term shall define plastid-containing material having a carotenoid content of 0.001% w/w or lower.

Having regard to the selection of suitable sources for yielding enzymes which cleave carotinoids, it is to be understood, that, in addition to the sequences of β-diox I from Drosophila and β-diox II from human (Homo sapiens), mouse (Mus musculus) and zebrafish (Danio rerio) as disclosed herein, all functional equivalent DNA molecules and fragments thereof such as e.g. sequences which are allelic variants or syngenic or synthetically modified (manufactured) with respect to the sequences set out in SEQ ID Nos. 1, 16, 18, and/or 20, and which code for enzymes or functional fragments thereof displaying the same desired activity of asymmetrically cleaving β-carotene to retinoids from existing organisms and which are substantially homologous to the partial or whole sequence of Drosophila melanogaster (SEQ ID No. 1), Mus musculus (SEQ ID No. 16), Danio rerio (SEQ ID No. 18), and/or Homo sapiens (SEQ ID No. 20) can easily be found by the person skilled in the art via e.g. conventional screening, isolated and suitably be used e.g. in securing expression of a β-diox II polypeptide or functional fragment thereof having the desired biological or enzymatic activity of specifically cleaving β-carotene and lycopene to form β-apocarotenal and β-ionone, and apolycopenals, respectively, or for use in the determination of the presence of nucleic acid(s) being characteristic for said polypeptide or functional fragment thereof. For example, by using the sequence information of Drosophila melanogaster (SEQ ID No. 1), vertebrate β-diox II homologues from Homo sapiens (SEQ ID No. 20), Danio rerio (SEQ ID No. 18), and Mus musculus (SEQ ID No. 16) could be identified by routine screening procedures known in the art and described hereinbelow in further detail, and are also encompassed by the present invention.

Thus, these DNA sequences are preferably selected from the group consisting of:

(a) the DNA sequence as set out in either SEQ ID No. 16 and/or SEQ ID No. 18 and/or SEQ ID No. 20, and complementary strands thereof; and

(b) the DNA sequences extending from position 115 to 141, 286 to 354, 1081 to 1104, and 1396 to 1461 of SEQ ID No. 16, or complementary strands thereof; and

(c) the DNA sequences extending from position 191 to 217, 362 to 430, 1160 to 1183, and 1472 to 1537 of SEQ ID No. 18, or complementary strands thereof, and

(d) the DNA sequences extending from position 175 to 201, 346 to 414, 1153 to 1176, and 1468 to 1533 of SEQ ID No. 20, or complementary strands thereof; and

(e) DNA sequences which hybridize under high-stringency conditions to the DNA sequences or complementary strands as defined in (a), (b), (c) and (d) or functional fragments thereof; and

(f) DNA sequences which would hybridize to the DNA sequences as defined in (a), (b), (c), (d) and (e), but for the degeneracy of the genetic code.

Stringency of hybridisation refers to conditions under which polynucleic acids hybrids are stable. Such conditions are evident to those of ordinary skill in the field. As known to those of skill in the art, the stability of hybrids is reflected in the melting temperature (Tₘ) of the hybrid which decreases approximately 1 to 1.5° C. with every 1% decrease in sequence homology. In general, the stability of a hybrid is a function of sodium ion concentration and temperature. Typically, the hybridisation reaction is performed under conditions of higher stringency, followed by washes of varying stringency.

As used herein, high stringency refers to conditions that permit hybridisation of only those nucleic acid sequences that form stable hybrids in 1 M Na⁺ at 65-68° C. High stringency conditions can be provided, for example, by hybridisation in an aqueous solution containing 6×SSC, 5×Denhardt's, 1% SDS (sodium dodecyl sulphate), 0.1 Na⁺ pyrophosphate and 0.1 mg/ml denatured salmon sperm DNA as non specific competitor. Following hybridisation, high stringency washing may be done in several steps, with a final wash (about 30 min) at the hybridisation temperature in 0.2-0.1×SSC, 0.1% SDS.

Moderate stringency refers to conditions equivalent to hybridisation in the above described solution but at about 60-62° C. In that case the final wash is performed at the hybridisation temperature in 1×SSC, 0.1% SDS.

Low stringency refers to conditions equivalent to hybridisation in the above described solution at about 50-52° C. In that case, the final wash is performed at the hybridisation temperature in 2×SSC, 0.1% SDS.

It is to be understood that these conditions may be adapted and duplicated using a variety of buffers, e.g. formamide-based buffers, and temperatures. Denhardt's solution and SSC are well known to those of skill in the art as are other suitable hybridisation buffers [see, e.g. Sambrook et al., Molecular Cloning, Cold Spring Habour Laboratory Press (1989), or Ausubel, et al., eds. (1990) Current Protocols in Molecular Biology, John Wiley & Sons, Inc.]. Optimal hybridisation conditions have to be determined empirically, as the length and the GC content of the probe also play a role.

In this context is should be mentioned that the term “a DNA sequence is substantially homologous” with respect to a β-diox II encoding DNA sequence refers to a DNA sequence which encodes an amino acid sequence which is at least 45%, preferably at least 60%, more preferably at least 75%, and most preferably at least 90% identical to the amino acid sequences of β-diox II of Mus musculus, Danio rerio, and/or of Homo sapiens as set out in SEQ ID Nos. 17, 19, and 21, respectively, and which represents a polypeptide or functional fragment thereof having the biological activity of specifically cleaving β-carotene to form β-apocarotenal, and/or having the capability of specifically binding to antibodies raised against a polypeptide or functional fragment according to the invention.

According to a preferred embodiment, these DNA sequences are in the form of cDNAs, genomic or manufactured (synthetic) DNA sequences and can be prepared prepared as known in the art (see e.g. Sambrook et al., s.a.) or e.g. as specifically described hereinbelow.

Given the guidance provided herein, the nucleic acids of the invention are obtainable according to methods well known in the art. For example, a DNA of the invention is obtainable by chemical synthesis, using polymerase chain reaction (PCR) or by screening a genomic library or a suitable cDNA library prepared from a source believed to possess β-diox II and to express it at a detectable level.

Chemical methods for synthesis of a nucleic acid of interest are known in the art and include triester, phosphite, phosphoramidite and H-phosphonate methods, PCR and other autoprimer methods as well as oligonucleotide synthesis on solid supports. These methods may be used if the entire nucleic acid sequence of the nucleic acid is known, or the sequence of the nucleic acid complementary to the coding strand is available. Alternatively, if the target amino acid sequence is known, one may infer potential nucleic acid sequences using known and preferred coding residues for each amino acid residue.

An alternative means to isolate the gene encoding β-diox II is to use PCR technology as described e.g. in section 14 of Sambrook et al., 1989. This method requires the use of oligonucleotide probes that will hybridise to β-diox II nucleic acid. Strategies for selection of oligonucleotides are described below.

Libraries are screened with probes or analytical tools designed to identify the gene of interest or the protein encoded by it. For cDNA expression libraries suitable means include monoclonal or polyclonal antibodies that recognise and specifically bind to β-diox II; oligonucleotides of about 20 to 80 bases in length that encode known or suspected β-diox II cDNA from the same or different species; and/or complementary or homologous cDNAs or fragments thereof that encode the same or a hybridising gene. Appropriate probes for screening genomic DNA libraries include, but are not limited to oligonucleotides, cDNAs or fragments thereof that encode the same or hybridising DNA; and/or homologous genomic DNAs or fragments thereof.

A nucleic acid encoding β-diox II may be isolated by screening suitable cDNA or genomic libraries under suitable hybridisation conditions with a probe, i.e. a nucleic acid disclosed herein including oligonucleotides derivable from the sequences set forth in SEQ ID Nos. 1, 16, 18 and/or 20. Suitable libraries are commercially available or can be prepared e.g. from cell lines, tissue samples, and the like.

As used herein, a probe is e.g. a single-stranded DNA or RNA that has a sequence of nucleotides that includes between 10 and 50, preferably between 15 and 30 and most preferably at least about 20 contiguous bases that are the same as (or the complement of) an equivalent or greater number of contiguous bases as set forth e.g. in SEQ ID Nos. 1, 16, 18, and/or 20. The nucleic acid sequences selected as probes should be of sufficient length and sufficiently unambiguous so that false positive results are minimised. The nucleotide sequences can be based on conserved or highly homologous nucleotide sequences or regions of β-diox II as already mentioned hereinbefore. The nucleic acids used as probes may be degenerate at one or more positions. The use of degenerate oligonucleotides may be of particular importance where a library is screened from a species in which preferential codon usage in that species is not known.

Preferred regions from which to construct probes include 5′ and/or 3′ coding sequences, sequences predicted to encode ligand binding sites, and the like. For example, either the full-length cDNA clones as disclosed herein, or fragments thereof, can be used as probes. Preferably, nucleic acid probes of the invention are labelled with suitable label means for ready detection upon hybridisation. For example, a suitable label means is a radiolabel. The preferred method of labelling a DNA fragment is by incorporating α^{32P} dATP with the Klenow fragment of DNA polymerase in a random priming reaction, as is well known in the art. Oligonucleotides are usually end-labelled with γ^{32P}-labelled ATP and polynucleotide kinase. However, other methods (e.g. non-radioactive) may also be used to label the fragment or oligonucleotide, including e.g. enzyme labelling, fluorescent labelling with suitable fluorophores and biotinylation.

After screening the library, e.g. with a portion of DNA including substantially the entire β-diox II-encoding sequence or a suitable oligonucleotide based on a portion of said or equivalent DNA, positive clones are identified by detecting a hybridisation signal; the identified clones are characterised by restriction enzyme mapping and/or DNA sequence analysis, and then examined, e.g. by comparison with the sequences set forth herein, to ascertain whether they include DNA encoding a complete β-diox II (i.e., if they include translation initiation and termination codons). If the selected clones are incomplete, they may be used to rescreen the same or a different library to obtain overlapping clones. If the library is genomic, then the overlapping clones may include exons and introns. If the library is a cDNA library, then the overlapping clones will include an open reading frame. In both instances, complete clones may be identified by comparison with the DNAs and deduced amino acid sequences provided herein.

In order to detect any abnormality of endogenous β-diox II, genetic screening may be carried out using the nucleotide sequences of the invention as hybridisation probes. Also, based on the nucleic acid sequences provided herein antisense- or ribozyme-type therapeutic agents may be designed.

It is envisaged that the nucleic acids of the invention can be readily modified by nucleotide substitution, nucleotide deletion, nucleotide insertion or inversion of a nucleotide stretch, and any combination thereof. Such mutants can be used e.g. to produce a β-diox II mutant that has an amino acid sequence differing from the β-diox II sequences as found in nature. Mutagenesis may be predetermined (site-specific) or random. A mutation which is not a silent mutation must not place sequences out of reading frames and preferably will not create complementary regions that could hybridise to produce secondary mRNA structure such as loops or hairpins.

Furthermore, the present invention envisages and enables the use of the sequence data provided herein to conduct relational and functional genomic studies. Relational studies are used as adjuncts to sequencing and mapping activities, and are designed to provide interesting, and potentially important, hints about biological function including e.g. homology searches, secondary structure correlations, differential cDNA screening, expression cloning, genetic linkage analysis, positional cloning and mutational analysis. In contrast to relational studies, functional studies generally make use of cells or animals to attempt a more direct correlation of sequence and biological function and include e.g. screening for phenotypic changes in systems such as yeast, flies, mitochondria, human tissues, mice, and frogs, using gene “knockouts” or other methods intended to control gene expression or protein action in order to provide information useful in relating sequences to function. These techniques as such are well-known in the art.

Use of the above approaches should preferably achieve one or more of the following criteria: (a) inhibition of the gene sequence should be sequence-specific in order to substantially eliminate false-positive results; (b) should have a broad based applicability, i.e. it should be possible to work with both high and low abundance genes, as well as with sequences whose product may be intracellular, membrane-associated, or extracellular; (c) should be applicable in models predictive of the (human) condition of interest; (d) should allow dose-response studies to be conducted e.g. in order to determine the dose at which the target is most affected; (e) the amount of information needed for target validation studies preferably should be minimal, i.e. the technique e.g. allows for dealing directly with ESTs without the former requirement of obtaining full-length gene sequences, promotor and other regulatory information, or protein sequence/structure; (f) should be useable in a high-throughput mode.

Accordingly, the present invention provides sufficient guidance to apply all approaches and techniques described above including “knockouts”, intracellular antibodies, aptamers, antisense oligonucleotides, and ribozymes. In a preferred embodiment of the present invention, β-diox-specific antisense oligonucleotides derived from any of the β-diox II sequences mentioned herein such as those set forth in either SEQ ID Nos. 1, 16, 18, and/or 20 can be used in dose-response studies in relevant models of retinoid/vitamin A deficiency during any stage of an organism's development. In a further preferred embodiment, use is made of specifically designed ribozymes which deliver optimized sequence-specific inhibition by manipulating elements inherent to their mechanism of action. For example, ribozymes can be designed to bind only to their targets, and by chosing a target sequence of 15 nucleotides—well within the informational limits of typical ESRs—there is assurance, on a statistical basis, that the target sequence will appear only once in the genome. Accordingly, the invention generally provides ribozymes specifically designed to interact only with its target which is expected to appear only once in the genome, ensuring a high degree of assurance that only the specific target has been inhibited. More particularly, the invention provides ribozymes which are uniquely equipped to deliver several types of important controls that can verify that inhibition of a specific mRNA target was the actual cause of alteration of β-diox II-mediated conditions or phenotypes. It is known, for example, that mutating the ribozyme's catalytic core renders it incapable of cleavage but still functional in terms of highly specific binding to its target. These “inactivated” ribozymes produce either no or substantially reduced target inhibition relative to the active ribozyme—making them a very effective negative control. Alternatively, the catalytic core can be maintained in its active form, but the target arms are modified such that they will not bind the target sequence. If nonspecific cleavage is occurring, such a construct should show activity. Since ribozymes contain noncontiguous binding arms, each of the ribozyme's two binding arms binds seperately and adds to ribozyme selectivity while maintaining specificity. Due to the low binding strength of such noncontiguous binding arms compared to e.g. contiguous antisense binding, any mismatches between the ribozyme and the target sequence will not be expected to bind effectively and thus allow the target to fall off before cleavage.

For the approaches and techniques as exemplified above, both the entire sequence as well as (functional) fragments thereof, in particular those described hereinbefore, can be used.

If required, nucleic acids encoding β-diox-related proteins or polypeptides can be cloned from cells or tissues according to established procedures using probes derived from β-diox II. In particular, such DNAs can be prepared by:

a) isolating mRNA from suitable cells or tisues, selecting the desired mRNA, for example by hybridisation with a DNA probe or by expression in a suitable expression system, and screening for expression of the desired polypeptide, preparing single-stranded cDNA complementary to that mRNA, then double-stranded cDNA therefrom, or

b) isolating cDNA from a cDNA library and selecting the desired cDNA, for example using a DNA probe or using a suitable expression system and screening for expression of the desired polypeptide, or

c) incorporating the double-stranded DNA of step a) or b) into an appropriate expression vector,

d) transforming appropriate host cells with the vector and isolating the desired DNA.

Polyadenylated messenger RNA (step a) is isolated by known methods. Isolation methods involve, for example, homogenizing cells in the presence of a detergent and a ribonuclease inhibitor, for example heparin, guanidinium isothiocyanate or mercaptoethanol, extracting the mRNA with a chloroform-phenol mixture, optionally in the presence of salt and buffer solutions, detergents and/or cation chelating agents, and precipitating mRNA from the remaining aqueous, salt-containing phase with ethanol, isopropanol or the like. The isolated mRNA may be further purified by centrifuging in a caesium chloride gradient followed by ethanol precipitation and/or by chromatographic methods, for example affinity chromatography, for example chromatography on oligo(dT) cellulose or on oligo(U) sepharose. Preferably, such purified total mRNA is fractionated according to size by gradient centrifugation, for example in a linear sucrose gradient, or chromatography on suitable size fractionation columns, for example on agarose gels.

The desired mRNA is selected by screening the mRNA directly with a DNA probe, or by translation in suitable cells or cell-free systems and screening the obtained polypeptides. The selection of the desired mRNA is preferably achieved using a DNA hybridisation probe, thereby avoiding the additional step of translation. Suitable DNA probes are DNAs of known nucleotide sequence consisting of at least 17 nucleotides derived from DNAs encoding β-diox II or a related protein. Alternatively, EST sequence information can be used to generate suitable DNA probes.

Synthetic DNA probes are synthesised according to known methods as detailed hereinbelow, preferably by stepwise condensation using the solid phase phosphotriester, phosphite triester or phosphoramidite method, for example the condensation of dinucleotide coupling units by the phosphotriester method. These methods are adapted to the synthesis of mixtures of the desired oligonucleotides by using mixtures of two, three or four nucleotides dA, dC, dG and/or dT in protected form or the corresponding dinucleotide coupling units in the appropriate condensation step as described by Y. Ike et al. (Nucleic Acids Research 11, 477, 1983).

For hybridisation, the DNA probes are labelled, for example radioactively labelled by the well known kinase reaction. The hybridisation of the size-fractionated mRNA with the DNA probes containing a label is performed according to known procedures, i.e. in buffer and salt solutions containing adjuncts, for example calcium chelators, viscosity regulating compounds, proteins, irrelevant DNA and the like, at temperatures favouring selective hybridisation, for example between 0° C. and 80° C., for example between 25° C. and 50° C. or around 65° C., preferably at around 20° lower than the hybrid double-stranded DNA melting temperature.

Fractionated mRNA may be translated in cells, for example frog oocytes, or in cell-free systems, for example in reticulocyte lysates or wheat germ extracts. The obtained polypeptides are screened for β-diox II activity or for reaction with antibodies raised against β-diox II or the β-diox II related protein, for example in an immunoassay, for example radioimmunoassay, enzyme immunoassay or immunoassay with fluorescent markers. Such immunoassays and the preparation of polyclonal and monoclonal antibodies are well known in the art and are applied accordingly. According to the invention there are provided polyclonal antibodies.

The preparation of a single-stranded complementary DNA (cDNA) from the selected mRNA template is well known in the art, as is the preparation of a double-stranded DNA from a single-stranded DNA. The mRNA template is incubated with a mixture of deoxynucleoside triphosphates, optionally radioactively labelled deoxynucleoside triphosphates (in order to be able to screen the result of the reaction), a primer sequence such as an oligo-dT residue hybridising with the poly(A) tail of the mRNA and a suitable enzyme such as a reverse transcriptase for example from avian myeloblastosis virus (AMV). After degradation of the template mRNA for example by alkaline hydrolysis, the cDNA is incubated with a mixture of deoxynucleoside triphosphates and a suitable enzyme to give a double-stranded DNA. Suitable enzymes are for instance a reverse transcriptase, the Klenow fragment of E. coli DNA polymerase I or T4 DNA polymerase. Usually, a hairpin loop structure formed spontaneously by the single-stranded cDNA acts as a primer for the synthesis of the second strand. This hairpin structure is removed by digestion with S1 nuclease. Alternatively, the 3′-end of the single-stranded DNA is first extended by homopolymeric deoxynucleotide tails prior to the hydrolysis of the mRNA template and the subsequent synthesis of the second cDNA strand.

In the alternative, double-stranded cDNA is isolated from a cDNA library and screened for the desired cDNA (step b). The cDNA library is constructed by isolating mRNA from suitable cells, for example chicken embryonic cells, human mononuclear leukocytes or human embryonic epithelial lung cells, and preparing single-stranded and double-stranded cDNA therefrom as described above. This cDNA is digested with suitable restriction endonucleases and incorporated into λ phage, for example λ charon 4A or λ gt11 following established procedures. The cDNA library replicated on nitrocellulose membranes is screened by using a DNA probe as described hereinbefore, or expressed in a suitable expression system and the obtained polypeptides screened for reaction with an antibody specific for the desired β-diox II.

A variety of methods are known in the art for the incorporation of double-stranded cDNA into an appropriate vector (step c). For example, complementary homopolymer tracts may be added to the double-stranded DNA and the vector DNA by incubation in the presence of the corresponding deoxynucleoside triphosphates and an enzyme such as terminal deoxynucleotidyl transferase. The vector and double-stranded DNA are then joined by base pairing between the complementary homopolymeric tails and finally ligated by specific joining enzymes such as ligases. Other possibilities are the addition of synthetic linkers to the termini of the double-stranded DNA, or the incorporation of the double-stranded DNA into the vector by blunt- or staggered-end ligation.

The transformation of appropriate host cells with the obtained hybrid vector (step d) and the selection of transformed host cells (step e) are well known in the art. Hybrid vectors and host cells may be particularly suitable for the production of DNA, or for the production of the desired β-diox II.

In addition to being useful for the production of recombinant β-diox II protein, these nucleic acids are also useful as probes, thus readily enabling those skilled in the art to identify and/or isolate nucleic acid encoding β-diox II. The nucleic acid may be unlabelled or labelled with a detectable moiety. Furthermore, the nucleic acids according to the invention are useful e.g. in a method determining the presence or even quantity of β-diox II specific nucleic acid, said method comprising hybridising the DNA (or RNA) encoding (or complementary to) β-diox II to test sample nucleic acid and determining the presence and, optionally, the amount of β-diox II. In another aspect, the invention provides a nucleic acid sequence that is complementary to, or hybridises under stringent conditions to, a nucleic acid sequence encoding β-diox II. These oligonucleotides can efficiently be used in antisense and/or ribozyme approaches, including gene therapy.

The invention also provides a method for amplifying a nucleic acid test sample comprising priming a nucleic acid polymerase (chain) reaction with nucleic acid (DNA or RNA) encoding (or complementary to) β-diox II.

The DNA-sequences of the present invention can thus be used as a guideline to define new PCR primers for the cloning of substantially homologous DNA sequences from other sources. In addition they and such homologous DNA sequences can be integrated into vectors by methods known in the art and described by e.g. Sambrook et al. (s.a.) to express or overexpress the encoded polypeptide(s) in appropriate host systems. However, a man skilled in the art knows that also the DNA-sequences themselves can be used to transform the suitable host systems of the invention to get overexpression of the encoded polypeptide.

As outlined above, the present invention thus provides specific DNA molecules as well as plasmid or vector systems comprising the same which comprise a DNA sequence within an operable expression cassette capable of directing production of a β-carotene dioxygenase II functionally active to direct production of retinoids from β-carotene. Preferably, said DNA molecules further comprise at least one selectable marker gene or cDNA operably linked to a constitutive, inducible or tissue-specific promoter sequence allowing its expression in bacteria, yeast, fungi, insect, animal or plant cells, seeds, tissues or whole organisms. If plastid-containing material is selected for transformation it is preferred that the the coding nucleotide sequence is fused with a suitable plastid transit peptide encoding sequence, both of which preferably are expressed under the control of a tissue-specific or constitutive promoter.

Polypeptides according to the invention include β-diox II and derivatives thereof which retain at least one common structural determinant of β-diox II.

“Common structural determinant” means that the derivative in question possesses at least one structural feature of β-diox II. Structural features includes possession of an epitope or antigenic site that is capable of cross-reacting with antibodies raised against a naturally occurring or denatured β-diox II polypeptide or fragment thereof, possession of amino acid sequence identity with β-diox II and features having common a structure/function relationship. Thus β-diox II as provided by the present invention includes splice variants encoded by mRNA generated by alternative splicing of a primary transcript, amino acid mutants, glycosylation variants and other covalent derivatives of β-diox II which retain the physiological and/or physical properties of β-diox II. Exemplary derivatives include molecules wherein the protein of the invention is covalently modified by substitution, chemical, enzymatic, or other appropriate means with a moiety other than a naturally occurring amino acid. Such a moiety may be a detectable moiety such as an enzyme or a radioisotope. Further included are naturally occurring variants or homologues of β-diox II found with a particular species, preferably a mammal. Such a variant or homologue may be encoded by a related gene of the same gene family, by an allelic variant of a particular gene, or represent an alternative splicing variant of the β-diox II gene.

Derivatives which retain common structural features can be fragments of β-diox II. Fragments of β-diox II comprise individual domains thereof, as well as smaller polypeptides derived from the domains. Preferably, smaller polypeptides derived from β-diox II according to the invention define a single feature which is characteristic of β-diox II. Fragments may in theory be almost any size, as long as they retain one feature of β-diox II. Preferably, fragments will be between 5 and 200 amino acids in length. Longer fragments are regarded as truncations of the full-length β-diox II and generally encompassed by the term “β-diox II”. Exemplary fragments of a β-diox II polypeptide are represented by the amino acid sequences extending from 39 to 47, 96 to 118, 361 to 368, and 466 to 487 of SEQ ID No. 17, from 55 to 63, 112 to 134, 378 to 385, and 482 to 503 of SEQ ID No. 19, and from 59 to 67, 116 to 138, 385 to 392, and 490 to 511 of SEQ ID No. 21, respectively.

Derivatives of β-diox II also comprise mutants thereof, which may contain amino acid deletions, additions or substitutions, subject to the requirement to maintain at least one feature characteristic of β-diox II. Thus, conservative amino acid substitutions may be made substantially without altering the nature of β-diox II, as may truncations from the 5′ or 3′ ends. Deletions and substitutions may moreover be made to the fragments of β-diox II comprised by the invention. β-diox II mutants may be produced from a DNA encoding β-diox II which has been subjected to in vitro mutagenesis resulting e.g. in an addition, exchange and/or deletion of one or more amino acids. For example, substitutional, deletional or insertional variants of β-diox II can be prepared by recombinant methods and screened for immuno-crossreactivity with the native forms of β-diox II.

The present invention also provides polypeptides and derivatives of β-diox II which retain at least one common antigenic determinant of β-diox II.

“Common antigenic determinant” means that the derivative in question possesses at least one antigenic function of β-diox II. Antigenic functions includes possession of an epitope or antigenic site that is capable of cross-reacting with antibodies raised against a naturally occurring or denatured β-diox II polypeptide or fragment thereof.

Derivatives which retain common antigenic determinants can be fragments of β-diox II, such as e.g. those described herein. Fragments of β-diox II comprise individual domains thereof, as well as smaller polypeptides derived from the domains. Preferably, smaller polypeptides derived from β-diox II according to the invention define a single epitope which is characteristic of β-diox II. Fragments may in theory be almost any size, as long as they retain one characteristic of β-diox II. Preferably, fragments will be between 5 and 500 amino acids in length. Longer fragments are regarded as truncations of the full-length β-diox II and generally encompassed by the term “β-diox II”.

The present invention provides processes for producing a β-diox II polypeptide comprising the steps of (a) expressing a polypeptide encoded by a DNA as outlined above in a suitable host, and (b) isolating said β-diox II polypeptide according to conventional techniques well known in the art. In addition, there is provided a protein which is obtained or obtainable by use of the aforementioned process.

Preferably, the protein or derivative thereof of the invention is provided in isolated form. “Isolated” means that the protein or derivative has been identified and is free of one or more components of its natural environment. Isolated β-diox II includes β-diox II in a recombinant cell culture. β-diox II present in an organism expressing a recombinant β-diox II gene, whether the β-diox II protein is “isolated” or otherwise, is included within the scope of the present invention.

If desired, the retinoids such as β-apocarotenal, β-ionone and apolycopenal formed in any of the described systems (bacteria, fungi, plant, animals etc.) can be further metabolised to retinol, retinyl esters, retinoic acids and their corresponding stereoisomers. Those modifications can be useful to improve the efficiency of the cleavage reaction and/or to accumulate a desired retinoid. The accumulation of a specific retinoid can be useful because retinoids exert different biological functions depending on their oxidative state (alcohol, aldehyde and acid) and in addition on their stereoisomeric form e.g. retinaldehyde/retinol in vision and retinoic acid in developmental processes and differentiation while retinyl esters are the normal storage of vitamin A in animals. The accumulation of a desired retinoid derivative can be achieved by the co-expression of retinoid modifying enzymes with β-diox II. With those functional combinations, e.g. the accumulation of retinyl esters can be achieved in plants and/or bacteria used as feed, food and/or feed- and food additives or the biosynthesis of a specific retinoid e.g. 9-cis retinoic acid, the ligand of the RXR transcription factors, can be achieved. Furthermore, the co-expression of retinoid binding proteins from animal origin may improve the yield of a desired retinoid.

According to a preferred embodiment of the present invention, the following enzymes or combinations of enzymes are co-expressed together with β-diox II. For example, if it is desired to convert retinaldehyde to retinol, alcohol dehydrogenase (e.g. AF059256) and/or retinaldehyd dehydrogenase/reductase (e.g. AW211228) can be used. In case retinyl esters are intended to be produced from retinol, retinol acyltransferase (e.g. AF071510) can be used. If retinoic acid shall be produced from retinaldehyde, retinaldehyde oxidase (e.g. AB017482) would be selected. Furthermore, if retinoid binding proteins are desired to be co-expresed, selection of Retinol binding protein (e.g. AJ236884) could be envisaged. Finally, different isomerases can be co-expressed which isomerase the all trans forms of the above compounds to the 13cis, 11cis, 9cis or 7 cis isomers.

In accordance with the subject invention, means and methods for the transformation of plant cells, seeds, tissues or whole plants as well as for the transformation of microorganisms such as yeast, fungi and bacteria are provided to produce transformants capable of mediating the synthesis of retinoids. According to another aspect of the present invention, said methods can also be used to modify the retinoid metabolism in animals.

The host material selected for transformation should express the gene(s) introduced, and is preferably homozygous for expression thereof. Generally, the gene will be operably linked to a promoter functionally active in the targeted host cells of the particular plant, insect, animal or microorganism (such as e.g. fungi including yeast and bacteria). The expression should be at a level such that the characteristic desired from the gene is obtained. For example, the expression of a selectable marker gene should provide for an appropriate selection of transformants yielded according to the methods of the present invention. Similarly, the expression of a gene coding for an enzyme displaying the desired activity of cleaving β-carotene to carotenoids/retinoids for enhanced nutritional quality should result in a transformant having a relatively higher content of the encoded gene product as compared to that of the same species which is not subjected to the transformation method according to the present invention. On the other hand, it will generally be desired to limit the excessive expression of the gene of interest in order to avoid significantly adversely affecting the normal physiology of the plant, insect, fungal, animal or microorganism, i.e. to the extent that cultivation thereof becomes difficult.

The gene encoding β-carotene dioxygenase II can be used in expression cassettes for expression in the transformed procaryotic or eucaryotic host cell, seed, tisue or whole organism. To achieve the objects of the present invention, i.e., to introduce the ability to cleave β-carotene to form retinoids in a target host of interest, the transformation is preferably carried out by use of an operable expression cassette comprising a transcriptional initiation region linked to the gene encoding β-carotene dioxygenase II.

The transcriptional initiation may be native or analogous to the host or foreign or heterologous to the host. By foreign is intended that the transcriptional initiation region is not found in the wild-type host into which the transcriptional initiation region is introduced.

In plant material, those transcriptional initiation regions are of particular interest which are associated with storage proteins, such as glutelin, patatin, napin, cruciferin, β-conglycinin, phaseolin, or the like.

The transcriptional cassette will include, in 5′-3′ direction of transcription, a transcriptional and translational initiation region, a DNA sequence encoding β-carotene dioxygenase II or a functional fragment thereof retaining its specific enzymatic, immunogenic or biological activity, and a transcriptional and translational termination region functional in the targeted host material such as, e.g., plants or microorganims, respectively. The termination region may be native with the transcriptional initiation region, may be native with the DNA sequence of interest, or may be derived from other sources. Convenient termination regions suitable for plant material are available from the Ti-plasmid of A. tumefaciens such as the octopine synthase and nopaline synthase termination regions [see also, Guerineau et al., (1991) Mol. Gen. Genet. 262, 141-144; Proudfoot, (1991) Cell 64, 671-674; Sanfacon et al., (1991) Gened Dev. 5, 141-149; Mogen et al., (1990) Plant Cell 2, 1261-1272; Munroe et al., (1990) Gene 91, 151-158; Ballas et al., (1989), Nucl. Acids Res. 17, 7891-7903; Joshi et al., (1987) Nucl. Acids Res. 15, 9627-9639].

For the expression of β-carotene dioxygenase II in plant or plastid-containing material, the coding sequence is preferably fused to a sequence encoding a transit peptide which after expression and translation directs the translocation of the protein upon cleavage of the transit peptide to (plant) plastids, such as chloroplasts, where the carotenoid biosynthesis takes place. For example, the β-diox II cDNA can be translationally fused to a sequence encoding for the transit peptide of the small subunit of ribulose-1,5-bis-phosphate carboxylase (rubisco) or to sequences coding for transit peptides of other plastid proteins. Such transit peptides are known in the art [see, for example, Von Heijne et al., (1991) Plant Mol. Biol. Rep. 9, 104-126; Clark et al., (1989) J. Biol Chem. 264, 17544-17550; Della-Cioppa et al., (1987) Plant Physiol. 84, 965-968; Romer et al., (1993) Biochim Biophys. Res. Commun. 196, 1414-1421; and, Shah et al., (1986) Science 233, 478-481]. Any genes useful for carrying out the present invention can utilize native or heterologous transit peptides.

The construct can also include any other necessary regulators such as plant translational consensus sequences (Joshi, 1987, s.a.), introns [Luehrsen and Walbot, (1991) Mol. Gen. Genet. 225, 81-93] and the like, operably linked to the nucleotide sequence encoding β-carotene dioxygenase II. Intron sequences within the coding gene desired to be introduced may increase its expression level by stabilizing the transcript and allowing its effective translocation out of the nucleus. Among the known such intron sequences are the introns of the plant ubiquitin gene (Cornejo, Plant Mol. Biol. 23, 567-581, 1993). Furthermore, it has been observed that the same construct inserted at different loci on the genome can vary in the level of expression in plants. The effect is believed to be due at least in part to the position of the gene on the chromosome, i.e., individual isolates will have different expression levels (see, for example, Hoever et al., Transgenic Res. 3, 159-166, 1994). Further regulatory DNA sequences that may be used for the construction of expression cassettes include, for example, sequences that are capable of regulating the transcription of an associated DNA sequence in plant tissues in the sense of induction or repression.

There are, for example, certain plant genes that are known to be induced by various internal and external factors, such as plant hormones, heat shock, chemicals, pathogens, oxygen deficiency, light, stress, etc.

A further group of DNA sequences which can be regulated comprises chemically-driven sequences that are present, e.g., in the PR (pathogenesis-related) protein genes of tobacco and are inducible by means of chemical regulators such as those described in EP-A 0 332 104.

Yet another consideration in expression of foreign genes in plants, animals, insects, fungi or microorganims is the level of stability of the transgenic genome, i.e., the tendency of a foreign gene to segregate from the population. If a selectable marker is linked to the gene or expression cassette of interest, then selection can be applied to maintain the transgenic host organism or part thereof.

It may be beneficial to include 5′ leader sequences in the expression cassette construct. Such leader sequences can act to enhance translation. Translation leaders are known in the art and include: picornavirus leaders, for example, EMCV leader (Encephalomyocarditis 5′ noncoding region; Elroy-Stein et al., Proc. Natl. Acad. Sci. USA 86, 6126-6130, 1989); potyvirus leaders, for example, TEV leader (Tobacco Etch Virus; Allisson et al., Virology 154, 9-20, 1986); and human immunoglobulin heavy-chain binding protein (BiP, Macejak and Sarnow, Nature 353, 90-94, 1991); untranslated leader from the coat protein mRNA of alfalfa mosaic virus (AMV RNA 4; Jobling and Gehrke, Nature 325, 622-625, 1987); tobacco mosaic virus leader (TMV; Gallie et al., Molecular Biology of RNA, 237-256, 1989); and maize chlorotic mottle virus leader (MCMV; Lommel et al., Virology 81, 382-385, 1991; see also, Della-Cioppa et al., 1987, s.a.).

Depending upon where the DNA sequence encoding β-carotene dioxygenase II is to be expressed, it may be desirable to synthesize the sequence with host preferred codons, or alternatively with chloroplast or plastid preferred codons. The plant preferred codons may be determined from the codons of highest frequency in the proteins expressed in the largest amount in the particular plant species of interest (see, EP-A 0 359 472; EP-A 0 386 962; WO 91/16432; Perlak et al., Proc. Natl. Acad. Sci 88, 3324-3328, 1991; and Murray et al., Nucl. Acids. Res. 17, 477-498, 1989). In this manner, the nucleotide sequences can be optimized for expression in any targeted host. It is recognized that all or any part of the gene sequence may be optimized or synthetic. That is, synthetic or partially optimized sequences may also be used. For the construction of chloroplast preferred genes, see U.S. Pat. No. 5,545,817.

Expression systems encoding β-diox II are useful for the study of β-diox II activity, particularly in the context of transgenic cells, tissues or animals. Preferred is a system in which β-diox II expression has been attenuated, particularly where this is achieved by means of transposon insertion. Mutant cells, tissues or animals according to the invention have impaired β-diox II expression. Especially those expression mutants in which expression is severely attenuated but not limited, are useful for the study of β-diox II activity. They show increased sensitivity to modulated interaction of putative upstream signalling agents with specific target domains of β-diox II, as well as modification of the downstream targets predicted to mediate its biological response. Thus, the invention also provides a method for assessing the ability of an agent to target β-diox II activity comprising exposing a β-diox II mutant as described herein to the agent, and judging the effect of the biological activity of β-diox II.

In preparing the transcription cassette, the various DNA fragments may be manipulated, so as to provide for the DNA sequences in the proper orientation and, as appropriate in the proper reading frame. Towards this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resection, ligation, or the like may be employed, where insertions, deletions or substitutions, e.g. transitions and transversions, may be involved.

The expression cassette carrying the cDNA or genomic DNA encoding native or mutant β-carotene dioxygenase II is placed into an expression vector by standard methods. As used herein, vector (or plasmid) refers to discrete elements that are used to introduce heterologous DNA into cells for either expression or replication thereof. Selection and use of such vehicles are well within the skill of the artisan. Many vectors are available, and selection of an appropriate vector will depend on the intended use of the vector, i.e. whether it is to be used for DNA amplification or for DNA expression, the size of the DNA to be inserted into the vector, the type of host (plant, animal, insect, fungi or microorganism) to be transformed with the vector, and the method of introducing the expression vector into host cells. Each vector contains various components depending on its function (amplification of DNA or expression of DNA) and the host cell for which it is compatible. A typical expression vector generally includes, but is not limited to, prokaryotic DNA elements coding for a bacterial replication origin and an antibiotic resistance gene to provide for the growth and selection of the expression vector in the bacterial host; a cloning site for insertion of an exogenous DNA sequence, which in this context would code for an enzyme capable of cleaving β-carotene to form carotenoids/retinoids; eukaryotic DNA elements that control initiation of transcription of the exogenous gene, such as a promoter; and DNA elements that control the processing of transcripts, such as a transcription termination/polyadenylation sequence. It also can contain such sequences as are needed for the eventual integration of the vector into the chromosome of the targeted host.

In a preferred embodiment, the expression vector also contains a gene encoding a selection marker such as, e.g,. hygromycin phosphotransferase (van den Elzen et al., Plant Mol. Biol. 5, 299-392, 1985), which is functionally linked to a promoter. Additional examples of genes that confer antibiotic resistance and are thus suitable as selectable markers include those coding for neomycin phosphotransferase kanamycin resistance (Velten et al., EMBO J. 3, 2723-2730, 1984); the kanamycin resistance (NPT II) gene derived from Tn5 (Bevan et al., Nature 304, 184-187, 1983); the PAT gene described in Thompson et al., (EMBO J. 6, 2519-2523, 1987); and chloramphenicol acetyltransferase. For a general description of plant expression vectors and selectable marker genes suitable according to the present invention, see Gruber et al., [in: Methods in Plant Molecular Biology and Biotechnology 89-119 (CRC Press), 1993]. As to a selective gene marker appropriate for yeast, any marker gene can be used which facilitates the selection for transformants due to the phenotypic expression of the marker gene. Suitable markers for yeast are, for example, those conferring resistance to antibiotics G418, hygromycin or bleomycin, or provide for prototrophy in an auxotrophic yeast mutant, for example the URA3, LEU2, LYS2, TRP1, or HIS3 gene.

Suitable selectable markers for mammalian cells are those that enable the identification of cells competent to take up β-diox nucleic acid, such as dihydrofolate reductase (DHFR, methotrexate resistance), thymidine kinase, or genes conferring resistance to G418 or hygromycin. The mammalian cell transformants are placed under selection pressure which only those transformants which have taken up and are expressing the marker are uniquely adapted to survive. In the case of a DHFR or glutamine synthase (GS) marker, selection pressure can be imposed by culturing the transformants under conditions in which the pressure is progressively increased, thereby leading to amplification (at its chromosomal integration site) of both the selection gene and the linked DNA that encodes β-diox II. Amplification is the process by which genes in greater demand for the production of a protein critical for growth, together with closely associated genes which may encode a desired protein, are reiterated in tandem within the chromosomes of recombinant cells. Increased quantities of desired protein are usually synthesised from thus amplified DNA.

A promoter element employed to control expression of the gene of interest and the marker gene, respectively, can be any plant-compatible promoter. Those can be plant gene promoters, such as the promoter for the small subunit of ribulose-1,5-bis-phosphate carboxylase (RUBISCO), or promoters from tumour-inducing plasmids of Agrobacterium tumefaciens, like that nopaline synthase and octopine synthase promoters, or viral promoters such as the cauliflower mosaic virus (CaMV) 19S and 35S promoters or the figwort mosaic virus 35S promoter. See international application WO 91/19806, for example, for a review of known plant promoters which are suitable for use in the present invention.

“Tissue-specific” promoters provide that accumulation of the desired gene product is particularly high in the tissue in which products of the carotenoid or xanthophyll biosynthetic pathway are expressed; although some expression may also occur in other parts of the plant. Examples of known tissue-specific promoters include the glutelin 1 promoter (Kim et al., Plant Cell Physiol. 34, 595-603, 1993; Okita et al., J. Biol. Chem 264, 12573-12581, 1989; Zheng et al., Plant J. 4, 357-366, 1993), the tuber-directed class I patatin promoter (Bevan et al., Nucl. Acid Res. 14, 4625-4638, 1986); the promoters associated with potato tuber ADPGPP genes (Muller et al., Mol. Gen. Genet 224, 136-146, 1990); the soybean promoter of β-conglycinin, also known as the 7S protein, which drives seed-directed transcription (Bray, Planta 172, 364-370, 1987); and seed-directed promoters from the zein genes of maize endosperm (Pedersen et al., Cell 29, 1015-1026, 1982). A further type of promoter which can be used according to the invention is a plant ubiquitin promoter. Plant ubiquitin promoters are well known in the art, as evidenced by Kay et al., (Science 236, 1299, 1987), and EP-A 0 342 926. Equally suitable for the present invention are actin promoters, histone promoters and tubulin promoters. Examples of preferred chemically inducible promoters, such as the tobacco PR-1a promoter, are detailed in EP-A 0 332 104. Another preferred category of promoters is that which is wound inducible. Preferred promoters of this kind include those described by Stanford et al., (Mol. Gen. Genet. 215, 200-208, 1989), Xu et al., (Plant Mol. Biol. 22, 573-588, 1993), Logemann et al., (Plant Cell 1, 151-158, 1989), Rohrmeier & Lehle, (Plant Mol. Biol. 22, 783-792, 1993), Firek et al., (Plant Molec. Biol. 22, 192-142, 1993), and Warner et al., (Plant J. 3, 191-201, 1993).

According to a preferred embodiment, the cassette for the expression of β-carotene dioxygenase II comprises the β-diox II cDNA translationally fused to a sequence encoding a transit peptide for plastid import, polyadenylation signals and transcription terminators, each operably linked to a suitable constitutive, inducible or tissue-specific promoter which enables the expression of the desired protein in plant cells, seeds, tissues or in whole plants.

Moreover, the β-diox II gene according to the invention preferably includes a secretion sequence in order to facilitate secretion of the polypeptide from bacterial hosts, such that it will be produced as a soluble native peptide rather than in an inclusion body. The peptide can be recovered from the bacterial periplasmic space, or the culture medium, as appropriate.

Suitable promoting sequences for use with yeast hosts may be regulated or constitutive and are preferably derived from a highly expressed yeast gene, especially a Saccharomyces cerevisiae gene. Thus, the promoter of the TRP1 gene, the ADHI or ADHII gene, the acid phosphatase (PH05) gene, a promoter of the yeast mating pheromone genes coding for the alpha- or a-factor or a promoter derived from a gene encoding a glycolytic enzyme such as the promoter of the enolase, glyceraldehyde-3-phosphate dehydrogenase (GAP), 3-phospho glycerate kinase (PGK), hexokinase, pyruvate decarboxylase, phosphofructokinase, glucose-6-phosphate isomerase, 3-phosphoglycerate mutase, pyruvate kinase, triose phosphate isomerase, phosphoglucose isomerase or glucokinase genes, or a promoter from the TATA binding protein (TBP) gene can be used. Furthermore, it is possible to use hybrid promoters comprising upstream activation sequences (UAS) of one yeast gene and downstream promoter elements including a functional TATA box of another yeast gene, for example a hybrid promoter including the UAS(s) of the yeast PH05 gene and downstream promoter elements including a functional TATA box of the yeast GAP gene (PH05-GAP hybrid promoter). A suitable constitutive PHO5 promoter is e.g. a shortened acid phosphatase PH05 promoter devoid of the upstream regulatory elements (UAS) such as the PH05 (-173) promoter element starting at nucleotide-173 and ending at nucleotide-9 of the PH05 gene. β-diox II gene transcription from vectors in mammalian hosts may be controlled by promoters derived from the genomes of viruses such as polyoma virus, adenovirus, fowlpox virus, bovine papilloma virus, avian sarcoma virus, cytomegalovirus (CMV), a retrovirus and Simian Virus 40 (SV40), from heterologous mammalian promoters such as the actin promoter or a very strong promoter, e.g. a ribosomal protein promoter, and from the promoter normally associated with β-diox sequence, provided such promoters are compatible with the host cell systems.

Transcription of a DNA encoding β-diox II by higher eukaryotes may be increased by inserting an enhancer sequence into the vector. Enhancers are relatively orientation and position independent. Many enhancer sequences are known from mammalian genes (e.g. elastase and globin). However, typically one will employ an enhancer from a eukaryotic cell virus. Examples include the SV40 enhancer on the late side of the replication origin (bp 100-270) and the CMV early promoter enhancer. The enhancer may be spliced into the vector at a position 5′ or 3′ to β-diox II DNA, but is preferably located at a site 5′ from the promoter.

Advantageously, a eukaryotic expression vector encoding β-diox II can comprise a locus control region (LCR). LCRs are capable of directing high-level integration site independent expression of transgenes integrated into host cell chromatin, which is of importance especially where the β-diox II gene is to be expressed in the context of a permanently-transfected eukaryotic cell line in which chromosomal integration of the vector has occurred, in vectors designed for gene therapy applications or in transgenic animals or other hosts disclosed herein or known in the art.

According to a preferred embodiment of the present invention, the expression cassettes and plasmid or vector systems disclosed herein additionally comprise nucleic acid sequences which encode specific retinoid modifying enzymes and/or retinoid binding proteins, preferably being co-expressed with the polypeptide according to the invention, as already outlined above

Suitable eukaryotic host cells for expression of β-diox II embrace fungi including yeast, insect, plant, animal, human, or nucleated cells from other multicellular organisms will also contain sequences necessary for the termination of transcription and for stabilising the mRNA Such sequences are commonly available from the 5′ and 3′ untranslated regions of eukaryotic or viral DNAs or cDNAs. These regions contain nucleotide segments transcribed as polyadenylated fragments in the untranslated portion of the mRNA encoding β-diox II.

The procaryotic or eucaryotic host cells, seeds, tissues and whole organisms contemplated in the context of the present invention may be obtained by any of several methods. Those skilled in the art will appreciate that the choice of method might depend on the type of host such as plant, i.e. monocot or dicot, targeted for transformation. Such methods generally include direct gene transfer, chemically-induced gene transfer, electroporation, microinjection (Crossway et al., BioTechniques 4, 320-334, 1986; Neuhaus et al., Theor. Appl. Genet. 75, 30-36, 1987), Agrobacterium-mediated gene transfer, ballistic particle acceleration using, for example, devices available from Agracetus, Inc., Madison, Wis., and Dupont, Inc., Wilmington, Del. (see, for example, Sanford et al., U.S. Pat. No. 4,945,050; and Mc Cabe et al., Biotechnology 6, 923-926, 1988), and the like.

One method for obtaining the present transformed plants or parts thereof is direct gene transfer in which plant cells are cultured or otherwise grown under suitable conditions in the presence of DNA oligonucleotides comprising the nucleotide sequence desired to be introduced into the plant or part thereof. The donor DNA source is typically a plasmid or other suitable vector containing the desired gene or genes. For convenience, reference is made herein to plasmids, with the understanding that other suitable vectors containing the desired gene are also contemplated.

Any suitable plant tissue which takes up the plasmid may be treated by direct gene transfer. Such plant tissue includes, for example, reproductive structures at an early stage of development, particularly prior to meiosis, and especially 1-2 weeks pre-meiosis. Generally, the pre-meiotic reproductive organs are bathed in plasmid solution, such as, for example, by injecting plasmid solution directly into the plant at or near the reproductive organs. The plants are then self-pollinated, or cross-pollinated with pollen from another plant treated in the same manner. The plasmid solution typically contains about 10-50 μg DNA in about 0.1-10 ml per floral structure, but more or less than this may be used depending on the size of the particular floral structure. The solvent is typically sterile water, saline, or buffered saline, or a conventional plant medium. If desired, the plasmid solution may also contain agents to chemically induce or enhance plasmid uptake, such as, for example, PEG, Ca²⁺ or the like.

Following exposure of the reproductive organs to the plasmid, the floral structure is grown to maturity and the seeds are harvested. Depending on the plasmid marker, selection of the transformed plants with the marker gene is made by germination or growth of the plants in a marker-sensitive, or preferably a marker-resistant medium. For example, seeds obtained from plants treated with plasmids having the kanamycin resistance gene will remain green, whereas those without this marker gene are albino. Presence of the desired gene transcription of mRNA therefrom and expression of the peptide can further be demonstrated by conventional Southern, northern, and western blotting techniques.

In another method suitable to carry out the present invention, plant protoplasts are treated to induce uptake of the plasmid or vector system according to the invention. Protoplast preparation is well-known in the art and typically involves digestion of plant cells with cellulase and other enzymes for a sufficient period of time to remove the cell wall. Typically, the protoplasts are separated from the digestion mixture by sieving and washing. The protoplasts are then suspended in an appropriate medium, such as, for example, medium F, CC medium, etc., typically at 10⁴-10⁷ cells/ml. To this suspension is then added the plasmid solution described above and an inducer such as polyethylene glycol, Ca²⁺, Sendai virus or the like. Alternatively, the plasmids may be encapsulated in liposomes. The solution of plasmids and protoplasts are then incubated for a suitable period of time, typically about 1 hour at about 25° C. In some instances, it may be desirable to heat shock the mixture by briefly heating to about 45° C., e.g. for 2-5 minutes, and rapidly cooling to the incubation temperature. The treated protoplasts are then cloned and selected for expression of the desired gene or genes, e.g. by expression of the marker gene and conventional blotting techniques. Whole plants are then regenerated from the clones in a conventional manner.

The electroporation technique is similar except that electrical current is typically applied to the mixture of naked plasmids and protoplasts, in an electroporation chamber in the absence or presence of polyethylene glycol, Ca²⁺ or the like. Typical electroporation includes 1-10 pulses of 40-10,000 DC volts for a duration of 1-2000 μs with typically 0.2 second intervals between pulses. Alternating current pulses of similar severity can also be used. More typically, a charged capacitor is discharged across the electroporation chamber containing the plasmid protoplast suspension. This treatment results in a reversible increase in the permeability of biomembranes and thus allows the insertion of the DNA according to the invention. Electroporated plant protoplasts renew their cell wall, divide and form callus tissue (see, for example, Riggs et al., 1986).

Another method suitable for transforming target cells involves the use of Agrobacterium. In this method, Agrobacterium containing the plasmid with the desired gene or gene cassette is used to infect plant cells and insert the plasmid into the genome of the target cells. The cells expressing the desired gene are then selected and cloned as described above. For example, one method for introduction of a gene of interest into a target tissue, e.g., a tuber, root, grain or legume, by means of a plasmid, e.g. an Ri plasmid and an Agrobacterium, e.g. A. rhizogenes or A. tumefaciens, is to utilize a small recombinant plasmid suitable for cloning in Escherichia coli, into which a fragment of T-DNA has been spliced. This recombinant plasmid is cleaved open at a site within the T-DNA. A piece of “passenger” DNA is spliced into this opening. The passenger DNA consists of the gene or genes of this invention which are to be incorporated into the plant DNA as well as a selectable marker, e.g., a gene for resistance to an antibiotic. This plasmid is then recloned into a larger plasmid and then introduced into an Agrobacterium strain carrying an unmodified Ri plasmid. During growth of the bacteria, a rare double-recombination will sometimes take place resulting in bacteria whose T-DNA harbours an insert: the passenger DNA. Such bacteria are identified and selected by their survival on media containing the antibiotic. These bacteria are used to insert their T-DNA (modified with passenger DNA) into a plant genome. This procedure utilizing A. rhizogenes or A. tumefaciens give rise to transformed plant cells that can be regenerated into healthy, viable plants (see, for example, Hinchee et al., 1988).

Another suitable approach is bombarding the cells with microprojectiles that are coated with the transforming DNA (Wang et al., Plant Mol. Biol. 11, 433-439, 1988), or are accelerated through a DNA containing solution in the direction of the cells to be transformed by a pressure impact thereby being finely dispersed into a fog with the solution as a result of the pressure impact (EP-A0 434 616).

Microprojectile bombardment has been advanced as an effective transformation technique for cells, including cells of plants. In Sanford et al., (Particulate Science and Technology 5, 27-37, 1987), it was reported that microprojectile bombardment was effective to deliver nucleic acid into the cytoplasm of plant cells of Allium cepa (onion). Christou et al., (Plant Physiol 87, 671-674, 1988) reported the stable transformation of soybean callus with a kanamycin resistance gene via microprojectile bombardment. The same authors reported penetration at approximately 0.1% to 5% of cells and found observable levels of NPTII enzyme activity and resistance in the transformed calli of up to 400 mg/l of kanamycin. McCabe et al., (1988, s.a.) report the stable transformation of Glycine max (soybean) using microprojectile bombardment. McCabe et al. further report the recovery of a transformed R₁ plant from an R₀ chimaeric plant (also see, Weissinger et al., Annual. Rev. Genet. 22,. 421-477, 1988; Datta et al., Biotechnology 8, 736-740, 1990 (rice); Klein et al., Proc. Natl. Acad. Sci. USA 85, 4305-4309, 1988 (maize); Klein et al., Plant Physiol. 91, 440-444, 1988 (maize); Fromm et al., Biotechnology 8, 833-839, 1990; and Gordon-Kamm et al., Plant Cell 2, 603-618, 1990 (maize).

Alternatively, a plant plastid can be transformed directly. Stable transformation of chloroplasts has been reported in higher plants, see, for example, Svab et al., (Proc. Natl. Acad. Sci. USA 87, 8526-8530, 1990); Svab and Maliga, (Proc. Natl. Acad Sci. USA 90, 913-917, 1993); Staub and Maliga, (EMBO J. 12, 601-606, 1993). The method relies on particle gun delivery of DNA containing a selectable marker and targeting of the DNA to the plastid genome through homologous recombination. In such methods, plastid gene expression can be accomplished by use of a plastid gene promoter or by trans-activation of a silent plastid-borne transgene positioned for expression from a selective promoter sequence such as recognized by T7 RNA polymerase. The silent plastid gene is activated by expression of the specific RNA polymerase from a nuclear expression construct and targeting the polymerase to the plastid by use of a transit peptide. Tissue-specific expression may be obtained in such a method by use of a nuclear-encoded and plastid-directed specific RNA polymerase expressed from a suitable plant tissue-specific promoter. Such a system has been reported in McBride et al., (Proc. Natl. Acad. Sci. USA 97, 7301-7305, 1994).

All plant transformation systems produce a mixture of transgenic and non-transgenic plants. The selection of transgenic plant cells can be accomplished by the introduction of an antibiotic or herbicide gene, enabling the transgenic plant cells to be selected on media containing the corresponding toxic compound. Besides those marker systems for the selection of transgenic plants new so-called “positive selection systems” have, been successfully used for plant transformation PCT/EP94/00575, WO94/20627). In contrast to antibiotic or herbicide resistance selection systems in which transgenic cells acquire the ability to survive on a selection medium while non-transgenic cells are killed, this method favours regeneration and growth of the transgenic plant cells while non-transgenic plant cells are starved, but not killed. Therefore, this selection strategy is termed “positive selection”. Vector systems for Agrobacterium-mediated transformation have been constructed and have been successfully used e.g. to transform potato, tobacco and tomato and are described e.g. by Haldrup, A., Petersen S. G. and Okkels F. T. [Plant Mol. Biol 37, pp. 287-296, (1998)]. Transformtion systems based on this positive selection systems can be used according to the invention to introduce constructs harbouring β-diox II to obtain plants expressing the β-diox II ploypeptide and are therefore enabled to the enzymatically cleavage of β-carotene to form β-apocarotenal. In addition, the use of those selection systems would have the advantage to overcome disadvantages in using antibiotic or herbicide genes in a selection system such as e.g. toxicity or allergenicity of the gene product and interference with antibiotic treatment, as generally known in the art.

The list of possible transformation methods given above by way of example is not claimed to be complete and is not intended to limit the subject of the invention in any way.

The present invention therefore also comprises a procaryotic or eucaryotic host cell, seed, tissue or whole organism transformed or transfected with the DNA molecule or with the plasmid or vector system according to the invention as set out hereinbefore in a manner enabling said host cell, seed, tissue or whole organism to express a polypeptide or functional fragment thereof having the biological activity of specifically cleaving β-carotene and lycopene to form β-apocarotenal and β-ionone, and apolycopenals, respectively, and/or having the capability of specifically binding to antibodies raised against said polypeptide or functional fragment thereof.

According to the invention, the procaryotic or eucaryotic host cell, seed, tissue or whole organism is selected from the group consisting of bacteria, yeast, fungi, insect, animal and plant cells, seeds, tissues or whole organisms. As for the procaryotic taxonomic groups, the host can be selected from the group consisting of proteobacteria including members of the alpha, beta, gamma, delta and epsilon subdivision, grain-positive bacteria including Actinomycetes, Firmicutes, Clostridium and relatives, flavobacteria, cyanobacteria, green sulfur bacteria, green non-sulfur bacteria, and archaea. Suitable proteobacteria belonging to the alpha subdivision can be selected from the group consisting of Agrobacterium, Rhodospirillum, Rhodopseudomonas, Rhodobacter, Rhodomicrobium, Rhodopila, Rhizobium, Nitrobacter, Aquaspirillum, Hyphomicrobium, Acetobacter, Beijerinckia, Paracoccus and Pseudomonas, with Agrobacterium and Rhodobacter being preferred and Agrobacterium aureus and Rhodobacter capsulatus, respectively, being most preferred. Suitable proteobacteria belonging to the beta subdivision can be selected from the group consisting of Rhodocyclus, Rhodopherax, Rhodovivax, Spirillum, Nitrosomonas, Spherotilus, Thiobacillus, Alcaligenes, Pseudomonas, Bordetella and Neisseria, with ammonia-oxidizing bacteria such as Nitrosomonas being preferred and Nitrosomonas sp. ENI-11 being most preferred. Suitable proteobacteria belonging to the gamma subdivision can be selected from the group consisting of Chromatium, Thiospirillum, Beggiatoa, Leucothrix, Escherichia and Azotobacter, with Enterobacteriaceae such as Escherichia coli being preferred, and with E. coli K12 strains such as e.g. M15 (described as DZ 291 by Villarejo et al. in J. Bacteriol. 120, 466-474, 1974), HB 101 (ATCC No. 33649) and E. coli SG13009 (Gottesman et al., J. Bacteriol. 148, 265-273, 1981) being most preferred. Suitable proteobacteria belonging to the delta subdivision can be selected from the group consisting of Bdellovibrio, Desulfovibrio, Desulfuromonas and Myxobacteria such as Myxococcus, with Myxococcus xanthus being preferred. Suitable proteobacteria belonging to the epsilon subdivision can be selected from the group consisting of Thiorulum, Wolinella and Campylobacter. Suitable gram-positive bacteria can be selected from the group consisting of Actinomycetes such as Actinomyces, Bifidobacterium, Propionibacterium, Streptomyces, Nocardia, Actinoplanes, Arthrobacter, Corynebacterium, Mycobacterium, Micromonospora, Frankia, Cellulomonas and Brevibacterium, and Firmicutes including Clostridium and relatives such as Clostridium, Bacillus, Desulfotomaculum, Thermoactinomyces, Sporosarcina, Acetobacterium, Streptococcus, Enterococcus, Peptococcus, Lactobacillus, Lactococcus, Staphylococcus, Rominococcus, Planococcus, Mycoplasma, Acheoleplasma and Spiroplasma, with Bacillus subtilis and Lactococcus lactis being preferred. Suitable flavobacteria can be selected from the group consisting of Bacteroides, Cytophaga and Flavobacterium, with Flavobacterium such as Flavobacterium ATCC21588 being preferred. Suitable cyanobacteria can be selected from the group consisting of Chlorococcales including Synechocystis and Synechococcus, with Synechocystis sp. and Synechococcus sp. PS717 being preferred. Suitable green sulfur bacteria can be selected from the group Chlorobium, with Chlorobium limicola f. thiosulfatophilum being preferred. Suitable green non-sulfur bacteria can be selected from the group Chloroflexaceae such as Chloroflexus, with Chloroflexus aurantiacus being preferred. Suitable archaea can be selected from the group of Halobacteriaceae including Halobacterium, with Halobacterium salinarum being preferred.

As for the eucaryotic taxonomic group of fungi including yeast, the host can be selected from the group consisting of Ascomycota including Saccharomycetes such as Pichia and Saccharomyces, and anamorphic Ascomycota including Aspergillus, with Saccharomyces cerevisiae and Aspergillus niger (e.g. ATCC 9142) being preferred.

The eucaryotic host sytem comprises insect cells which preferably are selected from the group consisting of SF9, SF21, Trychplusiani and M121. For example, the polypeptides according to the invention can advantageously be expressed in insect cell systems. Insect cells suitable for use in the method of the invention include, in principle, any lepidopteran cell which is capable of being transformed with an expression vector and expressing heterologous proteins encoded thereby. In particular, use of the Sf cell lines, such as the Spodoptera frugiperda cell line IPBL-SF-21 AE (Vaughn et al., (1977) In Vitro 13, 213-217) is preferred. The derivative cell line Sf9 is particularly preferred. However, other cell lines, such as Tricoplusia ni 368 (Kurstack and Marmorosch, (1976) Invertebrate Tissue Culture Applications in Medicine, Biology and Agriculture. Academic Press, New York, USA) can be employed. These cell lines, as well as other insect cell lines suitable for use in the invention, are commercially available (e.g. from Stratagene, La Jolla, Calif., USA). As well as expression in insect cells in culture, the invention also comprises the expression of heterologous proteins such as β-diox II in whole insect organisms. The use of virus vectors such as baculovirus allows infection of entire insects, which are in some ways easier to grow than cultured cells as they have fewer requirements for special growth conditions. Large insects, such as silk moths, provide a high yield of heterologous protein. The protein can be extracted from the insects according to conventional extraction techniques. Expression vectors suitable for use in the invention include all vectors which are capable of expressing foreign proteins in insect cell lines. In general, vectors which are useful in mammalian and other eukaryotic cells are also applicable to insect cell culture. Baculovirus vectors, specifically intended for insect cell culture, are especially preferred and are widely obtainable commercially (e.g. from Invitrogen and Clontech). Other virus vectors capable of infecting insect cells are known, such as Sindbis virus (Hahn et al., (1992) PNAS (USA) 89, 2679-2683). The baculovirus vector of choice (reviewed by Miller (1988) Ann. Rev. Microbiol. 42, 177-199) is Autographa californica multiple nuclear polyhedrosis virus, AcMNPV. Typically, the heterologous gene replaces at least in part the polyhedrin gene of AcMNPV, since polyhedrin is not required for virus production. In order to insert the heterologous gene, a transfer vector is advantageously used. Transfer vectors are prepared in E. coli hosts and the DNA insert is then transferred to AcMNPV by a process of homologous recombination.

The eucaryotic host sytem further comprises animal cells preferably selected from the group consisting of Baby Hamster Kidney (BHK) cells, Chinese Hamster Ovarian (CHO) cells, Human Embryonic Kidney (HEK) cells and COS cells, with NIH 3T3 and 293 being most preferred.

The host cells referred to in this disclosure comprise cells in in vitro culture as well as cells that are within a host organism.

The present invention also provides transgenic plant material, selected from the group consisting of protoplasts, cells, calli, tissues, organs, seeds, embryos, ovules, zygotes, etc. and especially, whole plants, that has been transformed by means of the method according to the invention and comprises the recombinant DNA of the invention in expressible form, and processes for the production of the said transgenic plant material.

As used herein, the term “plant” generally includes eukaryotic alga, embryophytes including Bryophyta, Pteridophyta and Spermatophyta such as Gymnospermae and Angiospermae, the latter including Magnoliopsida, Rosopsida (eu-“dicots”), Liliopsida (“monocots”). Representative and preferred examples include grain seeds, e.g. rice, wheat, barley, oats, amaranth, flax, triticale, rye, corn, and other grasses; oil seeds, such as oilseed Brassica seeds, cotton seeds, soybean, safflower, sunflower, coconut, palm, and the like; other edible seeds or seeds with edible parts including pumpkin, squash, sesame, poppy, grape, mung beans, peanut, peas, beans, radish, alfalfa, cocoa, coffee, hemp, tree nuts such as walnuts, almonds, pecans, chick-peas etc. Further examples comprise potatoes, carrots, sweet potatoes, sugar beets, tomato, pepper, cassava, willows, oaks, elm, maples, apples and bananas. Generally, the present invention is applicable in species cultivated for food, drugs, beverages, and the like. Preferably, the target plant selected for transformation is cultivated for food, such as, for example, grains, roots, legumes, nuts, vegetables, tubers, fruits, spices and the like.

Positive transformants generated according to the invention are regenerated into plants following procedures well-known in the art (see, for example, McCormick et al., Plant Cell Reports 5, 81-84, 1986). These plants may then be grown, and either pollinated with the same transformed strainer or different strains before the progeny can be evaluated for the presence of the desired properties and/or the extent to which the desired properties are expressed and the resulting hybrid having the desired phenotypic characteristic identified. A first evaluation may include, for example, the level of bacterial/fungal resistance of the transformed plants. Two or more generations may be grown to ensure that the subject phenotypic characteristic is stably maintained and inherited and then seeds harvested to ensure the desired phenotype or other property has been achieved.

Further comprised within the scope of the present invention are transgenic plants, in particular transgenic fertile plants transformed by means of the method of the invention and their asexual and/or sexual progeny, which still display the new and desirable property or properties due to the transformation of the mother plant.

The term ‘progeny’ is understood to embrace both, “asexually” and “sexually” generated progeny of transgenic plants. This definition is also meant to include all mutants and variants obtainable by means of known processes, such as for example cell fusion or mutant selection and which still exhibit the characteristic properties of the initial transformed plant, together with all crossing and fusion products of the transformed plant material.

Parts of plants, such as for example flowers, stems, fruits, leaves, roots originating in transgenic plants or their progeny previously transformed by means of the method of the invention and therefore consisting at least in part of transgenic cells, are also an object of the present invention. Another aspect of the present invention refers to diagnostic means and methods to measure, analyze and evaluate the qualitative and quantitative implications inherent to the nucleic and/or amino acid molecules according to the invention. For example, appropriately designed oligonucleotides specifically representative for the sequences disclosed herein can serve to enable e.g. tissue typing, expression profiling and allele determination (SNP analysis), preferably in the context of high throughput devices such as DNA and protein microarrays, and the like. Other fields of application comprise the manufacture of specific constructs generated as gene therapeutic tools, and the production of antibodies intended to be used e.g. for purification, therapeutic or diagnostic purposes.

In accordance with yet another embodiment of the present invention, there are provided antibodies specifically recognising and binding to β-diox II. For example, such antibodies may be generated against the β-diox II protein having the amino acid sequences set forth in SEQ ID Nos. 17, 19, or 21. Alternatively, β-diox II or β-diox II fragments (which may also be synthesised by in vitro methods), such as those described hereinbefore, are fused (by recombinant expression or an in vitro peptidyl bond) to an immunogenic polypeptide, and this fusion polypeptide, in turn, is used to raise antibodies against a β-diox II epitope.

Anti-β-diox II antibodies may be recovered from the serum of immunised animals. Monoclonal antibodies may be prepared from cells from immunised animals in the conventional manner.

The antibodies of the invention are useful for studying β-diox II localisation, screening of an expression library to identify nucleic acids encoding β-diox II or the structure of functional domains, as well as for the purification of β-diox II, and the like.

Antibodies according to the invention may be whole antibodies of natural classes, such as IgE and IgM antibodies, but are preferably IgG antibodies. Moreover, the invention includes antibody fragments, such as Fab, F(ab′)₂, Fv and ScFv. Small fragments, such Fv and ScFv, possess advantageous properties for diagnostic and therapeutic applications on account of their small size and consequent superior tissue distribution.

The antibodies according to the invention are especially indicated for diagnostic and therapeutic applications. Accordingly, they may be altered antibodies comprising an effector protein such as a toxin or a label. Especially preferred are labels which allow the imaging of the distribution of the antibody in a tumour in vivo. Such labels may be radioactive labels or radioopaque labels, such as metal particles, which are readily visualisable within the body of a patient Moreover, the may be fluorescent labels or other labels which are visualisable on tissue samples removed from patients.

Recombinant DNA technology may be used to improve the antibodies of the invention. Thus, chimeric antibodies may be constructed in order to decrease the immunogenicity thereof in diagnostic or therapeutic applications. Moreover, immunogenicity may be minimised by humanising the antibodies by CDR grafting [see EP-A 0 239 400 (Winter)] and, optionally, framework modification [see WO 90/07861 (Protein Design Labs)].

Antibodies according to the invention may be obtained from animal serum, or, in the case of monoclonal antibodies or fragments thereof, produced in cell culture. Recombinant DNA technology may be used to produce the antibodies according to established procedure, in bacterial or preferably mammalian cell culture. The selected cell culture system preferably secretes the antibody product.

Therefore, the present invention includes a process for the production of an antibody according to the invention comprising culturing a host, e.g. E. coli or a mammalian cell, which has been transformed with a hybrid vector comprising an expression cassette comprising a promoter operably linked to a first DNA sequence encoding a signal peptide linked in the proper reading frame to a second DNA sequence encoding the antibody, and isolating said antibody.

Multiplication of hybridoma cells or mammalian host cells in vitro is carried out in suitable culture media, which are the customary standard culture media, for example Dulbecco's Modified Eagle Medium (DMEM) or RPMI 1640 medium, optionally replenished by a mammalian serum, e.g. fetal calf serum, or trace elements and growth sustaining supplements, e.g. feeder cells such as normal mouse peritoneal exudate cells, spleen cells, bone marrow macrophages, 2-aminoethanol, insulin, transferrin, low density lipoprotein, oleic acid, or the like. Multiplication of host cells which are bacterial cells or yeast cells is likewise carried out in suitable culture media known in the art, for example for bacteria in medium LB, NZCYM, NZYM, NZM, Terrific Broth, SOB, SOC, 2×YT, or M9 Minimal Medium, and for yeast in medium YPD, YEPD, Minimal Medium, or Complete Minimal Dropout Medium.

In vitro production; provides relatively pure antibody preparations and allows scale-up to give large amounts of the desired antibodies. Techniques for bacterial cell, yeast or mammalian cell cultivation are known in the art and include homogeneous suspension culture, e.g. in an airlift reactor or in a continuous stirrer reactor, or immobilised or entrapped cell culture, e.g. in hollow fibres, microcapsules, on agarose microbeads or ceramic cartridges. Large quantities of the desired antibodies can also be obtained by multiplying mammalian cells in vivo. For this purpose, hybridoma cells producing the desired antibodies are injected into histocompatible mammals to cause growth of antibody-producing tumours. Optionally, the animals are primed with a hydrocarbon, especially mineral oils such as pristane (tetramethyl-pentadecane), prior to the injection. After one to three weeks, the antibodies are isolated from the body fluids of those mammals. For example, hybridoma cells obtained by fusion of suitable myeloma cells with antibody-producing spleen cells from Balb/c mice, or transfected cells derived from hybridoma cell line Sp2/0 that produce the desired antibodies are injected intraperitoneally into Balb/c mice optionally pretreated with pristane, and, after one to two weeks, ascitic fluid is taken from the animals.

The cell culture supernatants are screened for the desired antibodies, preferentially by immunofluorescent staining of cells expressing β-diox II, by immunoblotting, by an enzyme immunoassay, e.g. a sandwich assay or a dot-assay, or a radioimmunoassay.

For isolation of the antibodies, the immunoglobulins in the culture supernatants or in the ascitic fluid may be concentrated, e.g. by precipitation with ammonium sulphate, dialysis against hygroscopic material such as polyethylene glycol, filtration through selective membranes, or the like. If necessary and/or desired, the antibodies are purified by the customary chromatography methods, for example gel filtration, ion-exchange chromatography, chromatography over DEAE-cellulose and/or (immuno-)affinity chromatography, e.g. affinity chromatography with β-diox protein or with Protein-A.

The invention further concerns hybridoma cells secreting the monoclonal antibodies of the invention. The preferred hybridoma cells of the invention are genetically stable, secrete monoclonal antibodies of the invention of the desired specificity and can be activated from deep-frozen cultures by thawing and reckoning.

The invention also concerns a process for the preparation of a hybridoma cell line secreting monoclonal antibodies directed against β-diox II, characterised in that a suitable mammal, for example a Balb/c mouse, is immunised with purified β-diox II protein, an antigenic carrier containing purified β-diox II or with cells bearing β-diox II, antibody-producing cells of the immunised mammal are fused with cells of a suitable myeloma cell line, the hybrid cells obtained in the fusion are cloned, and cell clones secreting the desired antibodies are selected. For example spleen cells of Balb/c mice immunised with cells bearing β-diox II are fused with cells of the myeloma cell line PAI or the myeloma cell line Sp2/0-Ag14, the obtained hybrid cells are screened for secretion of the desired antibodies, and positive hybridoma cells are cloned.

Preferred is a process for the preparation of a hybridoma cell line, characterised in that Balb/c mice are immunised by injecting subcutaneously and/or intraperitoneally between 10 and 10⁷ and 10⁸ cells of human tumour origin which express β-diox II containing a suitable adjuvant several times, e.g. four to six times, over several months, e.g. between two and four months, and spleen cells from the immunised mice are taken two to four days after the last injection and fused with cells of the myeloma cell line PAI in the presence of a fusion promoter, preferably polyethylene glycol. Preferably the myeloma cells are fused with a three- to twentyfold excess of spleen cells from the immunised mice in a solution containing about 30% to about 50% polyethylene glycol of a molecular weight around 4000. After the fusion the cells are expanded in suitable culture media as described hereinbefore, supplemented with a selection medium, for example HAT medium, at regular intervals in order to prevent normal myeloma cells from overgrowing the desired hybridoma cells.

The invention also concerns recombinant DNAs comprising an insert coding for a heavy chain variable domain and/or for a light chain variable domain of antibodies directed to the β-diox II protein. By definition such DNAs comprise coding single stranded DNAs, double stranded DNAs consisting of said coding DNAs and of complementary DNAs thereto, or these complementary (single stranded) DNAs themselves.

Furthermore, DNA encoding a heavy chain variable domain and/or for a light chain variable domain of antibodies directed against β-diox II can be enzymatically or chemically synthesised DNA having the authentic DNA sequence coding for a heavy chain variable domain and/or for the light chain variable domain, or a mutant thereof. A mutant of the authentic DNA is a DNA encoding a heavy chain variable domain and/or a light chain variable domain of the above-mentioned antibodies in which one or more amino acids are deleted or exchanged with one or more other amino acids. Preferably said modification(s) are outside the CDRs of the heavy chain variable domain and/or of the light chain variable domain of the antibody. Such a mutant DNA is also intended to be a silent mutant wherein one or more nucleotides are replaced by other nucleotides with the new codons coding for the same amino acid(s). Such a mutant sequence is also a degenerated sequence. Degenerated sequences are degenerated within the meaning of the genetic code in that an unlimited number of nucleotides are replaced by other nucleotides without resulting in a change of the amino acid sequence originally encoded. Such degenerated sequences may be useful due to their different restriction sites and/or frequency of particular codons which are preferred by the specific host, particularly E. coli, to obtain an optimal expression of the heavy chain murine variable domain and/or a light chain murine variable domain.

The term “mutant” is intended to include a DNA mutant obtained by in vitro mutagenesis of the authentic DNA according to methods known in the art.

For the assembly of complete tetrameric immunoglobulin molecules and the expression of chimeric antibodies, the recombinant DNA inserts coding for heavy and light chain variable domains are fused with the corresponding DNAs coding for heavy and light chain constant domains, then transferred into appropriate host cells, for example after incorporation into hybrid vectors.

In the case of a diagnostic composition, the antibody is preferably provided together with means for detecting the antibody, which may be enzymatic, fluorescent, radioisotopic or other means. The antibody and the detection means may be provided for simultaneous, simultaneous separate or sequential use, in a diagnostic kit intended for diagnosis.

For example, the present invention provides a method of diagnosing a pathology which is characterized by an increased or decreased level of β-diox II in a given subject or individual. For example, a test sample is obtained and can be contacted with a reagent that can specifically bind β-diox II or with a nucleotide sequence that can bind to a nucleic acid molecule encoding β-diox II under suitable conditions, which allow specific binding of said reagent or said nucleotide sequence to said β-diox II target amino acid or nucleic acid sequence. Subsequently, the amount of said specific binding in said test sample can be compared with the amount of specific binding in a control sample, wherein an increased or decreased amount of said specific binding in said test sample as compared to said control sample is diagnostic of a pathology which is associated with the β-diox II-induced pathway.

The invention further provides methods of increasing or decreasing the amount of β-diox II in a cell or tissue, which can modulate the level of vitamin A or other retinoids. For example, the amount of β-diox II in a given target cell or tissue can be increased by introducing into the cell or tissue a nucleic acid molecule comprising a nucleic acid sequence encoding β-diox II or functional fragments thereof. Increasing the amount of β-diox II in a cell or tissue can induce or promote carotenoid/retinoid accumulation which will not only be beneficial for human beings but also for animals and feedstock which are frequently given vitamin preparations to improve nutrition quality.

### DEPOSITION OF BIOLOGICAL MATERIAL

E. coli cells carrying the gene encoding β-carotene dioxygenase derived from Drosophila melanogaster have been deposited under the Budapest Treaty with the Deutsche Sammlung von Mikroorganismen und Zellkulturen (DSMZ) in Braunschweig, Germany, under the identification reference ‘beta-diox’ and received the Accession No. DSM 13304.

The following examples are illustrative but not limiting of the present invention.

### EXAMPLES

### Plasmid Constructs

Construction of a β-carotene Accumulating E. coli Strain.

A plasmid carrying the genes for β-carotene biosynthesis from Erwinia herbicola was constructed using the vector pFDY297. pFDY297 is a derivative of pACYC177 (bp 486-3130) in which bp 1-485 from pBluescriptSK has been introduced. For cloning the genes for β-carotene biosynthesis from E. herbicola suitable endonuclease restriction sites were introduced at both ends of the PCR-product. First the crtE gene was inserted in pFDY297. CrtE was amplified by PCR from the plasmid pBL376 (Hundle, B. S., et al., (1994) Mol. Gen. Genet. 245, 406-416), which encodes the whole gene cluster for carotenoid biosynthesis from E. herbicola, using the primers: 5′-GCGTCGACCGCGGTCTACGGTTAACTG-3′ (SEQ ID No. 3) and 5′-GGGGTACCCTTGAACCCAAAAGGGCGG-3′ (SEQ ED No. 4) and the Expand PCR System (Boehringer, Mannheim, Germany). The PCR-product was digested with KpnI and SalI and ligated into the appropriate sites of pFDY297, resulting in the plasmid pCRTE. The genes crtB, crtI and crtY were amplified by PCR from pBL376 using the primers 5′-GCTCTAGACGTCTGGCGACGGCCCGCCA-3′ (SEQ ID No. 5) and 5′-GCGTCGACACCTACAGGCGATCCTGCG-3′ (SEQ ID No. 6) and the Expand PCR System (Boehringer, Mannheim, Germany). The PCR-product was digested with XbaI and SalI and ligated into the appropriate sites of pCRTE, resulting in the plasmid pORANGE. After transformation of the plasmid into E. Coli JM109, the resultant strain was able to synthesize β-carotene.

Cloning of β-diox from Drosophila melanogaster

We isolated total RNA from heads of adult flies obtained by hand dissection. Reverse transcription was performed using an oligo(T)-adapter primer 5′-GACCACGCGTATCGATGTCGACTTTTTTTTTTTTTTTTTT-3′ (SEQ ID No. 7) and Superscript reverse transcriptase (Gibco, Germany). For cloning of the full-length cDNA, PCR was performed with a specific up-primer 5′-GCAGCCGGTGTCTTCAAGAG-3′ (SEQ ID No. 8) derived from the published EST-fragment (Acc.AI063857) and an anchor primer 5′-GACCACGCGTATCGATGTCGA-3′ (SEQ ID No. 9) for the 3′-end and the Expand PCR System (Boehringer, Mannheim, Germany). The PCR-products obtained were isolated after separating on a 0.8% agarose gel and were directly ligated into the vector pBAD-TOPO (Invitrogen, Netherlands) and transformed into the β-carotene accumulating E. coli strain. Using this cloning strategy the Drosophila eDNA is translationally fused to a short open reading frame of the vector and is under the control of a positively regulated promoter which is inducible by L-arabinose. The bacteria were plated on LB agar with ampicillin (100 μg/ml), kanamycin (50 μg/ml) and L-arabinose (0.2%/). Positive colonies were identified by their fading from yellow to almost white. To analyze the resultant plasmid pβdiox and confirm its structure, both strands were completely sequenced.

Expression, Purification and Enzymatic Activity of β-diox-gex

For expression of β-diox the cDNA was amplified using the primers Gex-up: 5′-GGAATTCGCAGCCGGTGTCTTCAAGAG-3′ (SEQ ID No. 10) and Gex-down: 5′-CCTCGAGGTAGTCTTCCCATATAAGG-3′ (SEQ ID No. 11) and the Expand PCR System (Boehringer, Mannheim, Germany). With the oligonucleotide primers suitable restriction sites were introduced at both ends of the PCR-product. After restriction with EcoRI and NcoI the PCR-product was cloned into the appropriate sites of the expression vector pGEX-4T-1 (Pharmacia, Freiburg, Germany). The resultant plasmid pβdiox-gex was transformed into the E. coli strain JM109. Expression of the fusion protein β-diox-gex in E. coli and subsequent purification on glutathione sepharose 4B (Pharamacia, Freiburg, Germany) were carried out as described by the manufacturer's protocol.

Determination of β-diox Enzymatic Activity

The purified protein was incubated in a buffer containing 50 mM tricine/NaOH (pH 7.6) and 100 mM NaCl with 0.05% Triton-X-100 in a volume of 300 μl. To start the reaction, 5 μl of β-carotene (80 μM) was added dissolved in ethanol. For incubation in the presence of FeSO₄/ascorbate the compounds were added to a final concentration of 5 μM FeSO₄ and 10 mM L-ascorbat. After incubation for 2 h at 30° C., the reaction was stopped by the addition of 100 μl 2 M NH₂OH (pH 6.8) and 200 μl of methanol. Extraction and HPLC-analyses were carried out as described above.

Determination of mRNA-Levels in Different Parts of the Body by RT-PCR

Total RNA was isolated from adult flies (males and females). The body parts head, thorax and abdomen were obtained by hand dissection (legs and wings had been removed before). For measuring the steady state mRNA amounts of β-diox, RT-PCR was performed as described (von Lintig, J., et al., (1997) Plant J. 12, 625-634). Reverse transcription was performed with an oligo-(dT₁₇)-primer and Superscript reverse transcriptase (Gibco, Germany). PCRs were carried out using the primers [up-primer: 5′-CTGCAAACGGACCGACCACGT-3′ (SEQ ID No. 12), down primer: 5′-GCAAATCTATCGAAGATCGAG-3′ (SEQ ID No. 13)] for β-diox and Taq-polymerase (Pharmacia, Freiburg, Germany). As an internal control the mRNA level of the constitutively expressed ribosomal protein rp49 was investigated using intron-spanning primers [up-primer: 5′-GACTTCATCCGCCACCAGTC-3′ (SEQ ID No. 14) and down-primer 5′-CACCAGGAACTTCTTGAATCCG-3′ (SEQ ID No.15)]. The PCR was performed as two separate primer assays for β-diox and for rp49 as well as with all four primers combined in one assay.

Extraction of β-carotene and Retinoids from E. coli and HPLC-Analysis

The E. coli strains were grown under red safety light in 50 ml flasks in LB-medium until the cultures had reached an OD₆₀₀ of 1. Expression of β-diox was induced by the addition of L-arabinose (0.2% w/v) for 6 h or 16 h. Then the bacteria were harvested by centrifugation. The pellets were extracted by the following protocols: A. The pellet was resuspended in 200 μl 6 M formaldehyde and incubated for 2 min at 30° C., then 2 ml of dichloromethane was added. The carotenes and retinoids were extracted three times with 4 ml n-hexane. The collected organic phases were evaporated and dissolved in the HPLC-solvent. B. The pellet was resuspended in 2 ml 1 M NH₂OH in 50% methanol and incubated for 10 min at 30° C. Extraction was performed three times with petroleum ether. The collected organic phases were dried under a stream of N₂ and dissolved in the HPLC-solvent. HPLC-analyses was performed on a Hypersil 3 μm (Knaur, Germany) on a System Gold (Beckman) equipped with a multi-diode-array (model 166, Beckman) and the System Gold Nouveau software (Beckman, USA). The HPLC-solvent A (n-hexane/ethanol 99.75:0.25) was used for retinals and B (n-hexane/ethanol 99.5:05) for retinaloximes. The reference substances all-trans, 13-cis and 9-cis retinals were purchased from Sigma (Germany); 11-cis retinal was isolated from dark-adapted bovine eyes. The corresponding retinols and oximes were obtained by reducing with NaBH₄ or reaction with NH₂OH, respectively. For quantification of the molar amounts peak integrals were scaled with defined amounts of reference substances.

Preparation of Total RNA from Different Tissues of Mice

For the experiments 7 weeks old BALB/c mice (male and female) were sacrified, different tissues (colon, small intestine, stomach, spleen, brain, liver, heart, kidney, lung and testis) were dissected by hand and frozen immediately in liquid nitrogen. 50-100 mg of each tissue was homogenized with a pestle in a mortar with liquid nitrogen and total RNA was isolated using the RNeasy Kit (Qiagen, Hilden, Germany). The concentrations of the isolated total RNA were determined spectrophotometrically.

Cloning of cDNAs Encoding β-diox Homologous Proteins from Mouse

For cloning of full-length cDNAs encoding putative mouse β-carotene dioxygenases, RACE-PCRs were performed using a 5′/3′ RACE Kit (Roche Molecular Biochemicals, Mannheim, Germany). Reverse transcription was carried out using 500 ng of total RNA isolated from liver and an oligo-dT-anchor primer and Superscript reverse transcriptase (Life Technologies Inc.). For PCR an anchor primer and a specific up-primer were used: 5′-ATGGAGATAATATTTGGCCAG-3′ (SEQ ID No. 22) for the β,β-carotene-15,15′ dioxygenase (β-diox I) and 5′-ATGTTGGGACCGAAGCAAAGC-3′(SEQ ID No. 24) for β-diox II, respectively, and the Expand PCR System (Roche Molecular Biochemicals) were used. The PCR products were ligated into the vector pBAD-TOPO (Invitrogen, The Netherlands), resulting in the plasmids pDiox I and pDiox II.

Tissue Specific Expression of β-carotene Dioxygenases in Mouse

With total RNA (100 ng) isolated from different tissues RT-PCR was performed as has been described (von Lintig, J., Welsch, R., Bonk, M., Giuliano, G., Batschauer, A, and Kleinig, H. (1997) Plant J. 12, 625-634). The following sets of primers were used. β-diox I: up: 5′-ATGGAGATAATATTTGGCCAG-3′ (SEQ ID No. 22), and down: 5′-AACTCAGACACCACGATTC-3′(SEQ ID No.23); β-diox II: up: 5′-ATGTTGGGACCGAAGCAAAGC-3′ (SEQ ID No. 24), and down: 5′-TGTGCTCATGTAGTAATCACC-3′ (SEQ ID No.25). As a control for the intactness of the individual RNA samples the mRNA of β-actin was analyzed using the primers: up: 5′-CCAACCGTGAAAAGATGACCC-3′ (SEQ ID No. 26) and down: 5′-CAGCAATGCCTGGGTACATGG-3′ (SEQ ID No. 27).

Determination of the Enzymatic Activity In Vitro

For heterologous expression of the β-diox II polypeptide the plasmid pDiox It was transformed in the E. coli strain XL1-blue (Stratagene Inc.). The bacterial culture was grown at 28° C. until it reached an A₆₀₀ of 1.0. Then, L-(+)-arabinose were added to a final concentration of 0.8% (w/v) and the bacteria were cultivated for additional three hours. After harvesting the bacteria, they were broken with a French press in a buffer containing 50 mM Tricine/KOH (pH 7.6), 100 mM NaCl, and 1 mM Dithiothreitol. The crude extract was centrifuged at 20,000×g for 20 min. The supernatant was dialyzed against the same buffer for one hour at 4° C. Enzymatic activity was determined in crude extracts (100 μg of total protein) as described (Nagao, A., During, A., Hoshino, C., Terao, J., Olson, J. A. (1996) Arch. Biochem. Biophys. 328, 57-63) by adding β-carotene in micelles of Tween40 with a final concentration of 300 μM β-carotene and 0.2% Tween40 in the assay. Then, the lipophilic compounds were extracted and subjected to HPLC-analysis as described (von Lintig, J., and Vogt, K. (2000) J. Biol. Chem. 275, 11915-11920).

HPLC-Analysis of β-carotene and Lycopene Accumulating E. coli Strains Expressing the Two Different β-carotene Dioxygenases from Mouse

The plamids pDiox I and pDiox II were transformed into the appropriate E. coli strain. Growing conditions and analysis of the carotenes and their cleavage products were as previously described (von Lintig, J., and Vogt, K. (2000) J. Biol. Chem. 275, 11915-11920).

Mass Spectroscopy of the Cleavage Products by LC-MS and GC-MS

The E. coli strains were cultivated overnight and the bacteria were harvested by centrifugation. For solid phase extraction a SPME-syringe (100 μm PDMS, Supelco, Deisenhofen, Germany) was incubated in the supernatant for 15 min. Then, the compounds absorbed to the solid phase were subjected directly to GC-MS (GC: Hewlett-Packard 6890; MS: Hewlett-Packard 5973 (70 eV), Waldbronn, Germany) with a temperature program starting at 100° C. and increasing 6° C./min to 300° C. As column a DB-1 (30 m×0.25 mm×0.25 μm film thickness, J & W, Folsom, Canada) was used with helium as the carrier gas. For LC-MS analysis the bacterial pellet was extracted in the presence of hydroxylamine as previously described (von Lintig, J., and Vogt, K. (2000) J. Biol. Chem. 275, 11915-11920). LC/MS was run on an HP1100 HPLC module system (Hewlett Packard; Waldbronn, Germany), coupled to a Micromass (Manchester, UK) VG platform II quadrupole mass spectrometer equipped with an APcI interface (atmospheric pressure chemical ionization). UV absorbance was monitored with a diode array detector (DAD). MS parameters (APcI⁺-mode) were as follows: source temperature, 120° C.; APcI probe temperature, 350° C.; corona, 3.2 kV; high voltage lens, 0.5 kV; cone voltage, 30 V. The system was operated in full scan mode (m/z 250-1000). For data acquisition and processing, MassLynx 3.2 software was used. For peak separation, a Nucleosil RP-C18 column (5 μm, 250×4.6 mm) from Bischoff (Leonberg, Germany) was employed and kept at 25° C. The mobile phases consisted of a mixture of acetonitrile and methanol at 85:15, v/v (A) and isopropanol (B); gradient (% A [min]): 100 (8)-70 (10)-70 (25)-100 (28)-100 (32); flow rate, 1 mL/min; injection volume, 20 μL.

Sequence Comparison and Phylogenetic Tree Analysis

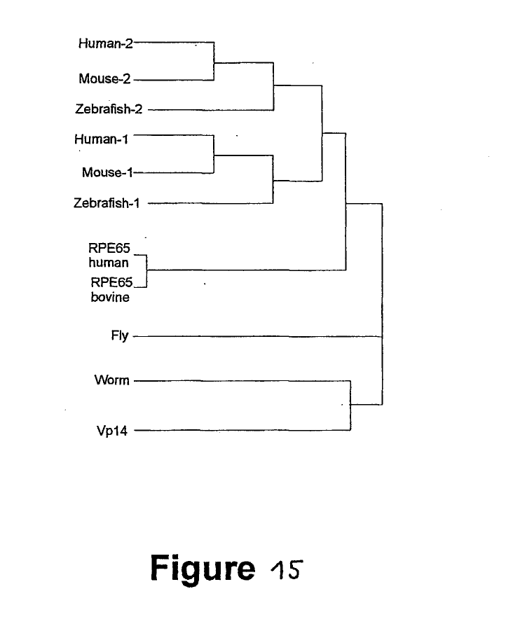

Vector NTI Suite 6.0 (InforMax Inc, Oxford, United Kingdom) was used and lead to the results as shown in FIG. 15.

Chemicals used were: β-ionone (Roth, Karlsruhe, Germany), 12′-β-apocarotenal (BASF, Ludwigshafen, Germany), and 8′-β-apocarotenal (Sigma, Deisenhofen, Germany).

### Results

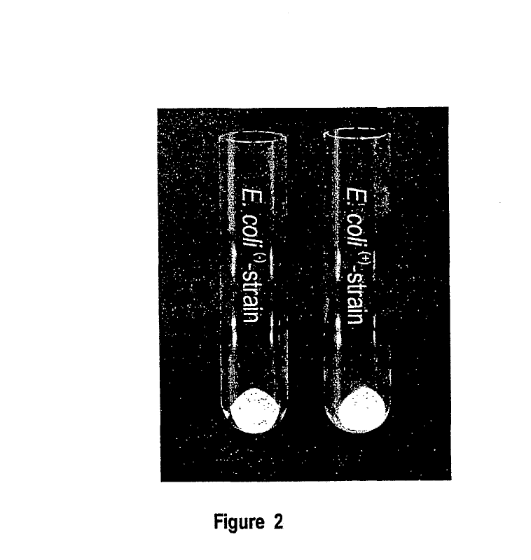

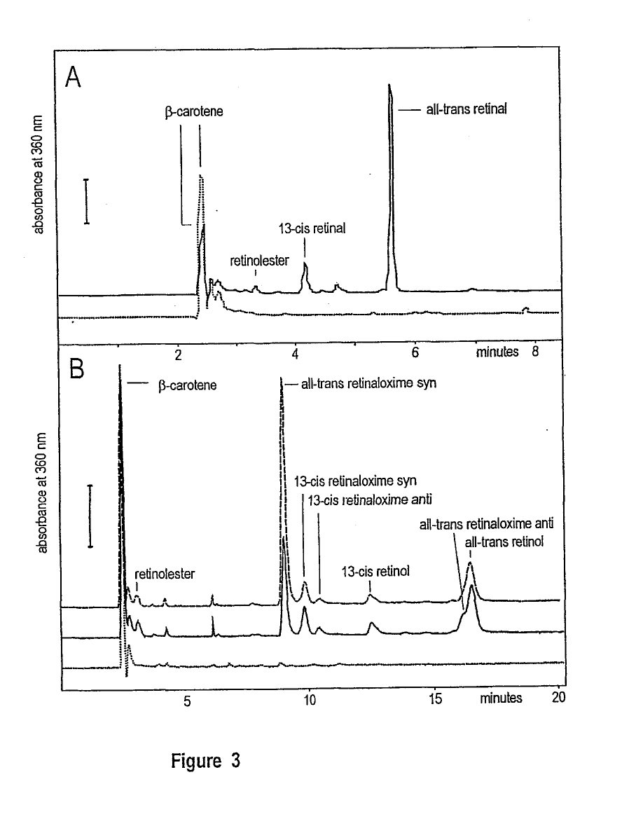

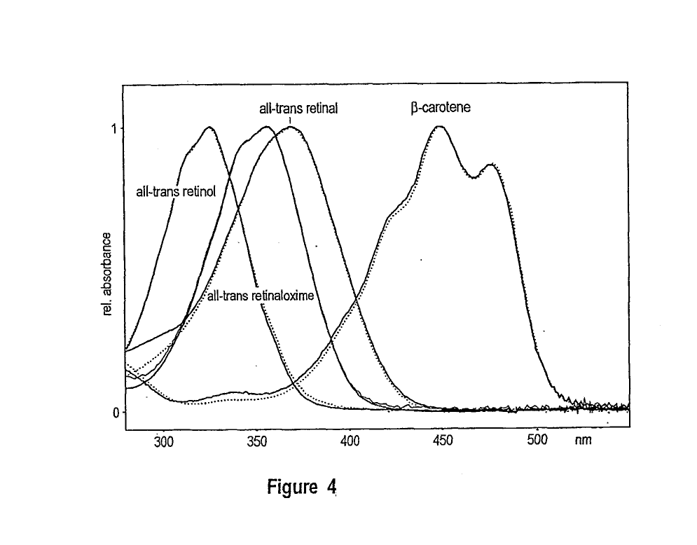

In order to find homologues of vp14, the plant carotenoid cleaving enzyme, insect EST-libraries were searched and a published EST-fragment from Drosophila melanogaster (Acc.AI063857) was discovered. For cloning of the full length cDNA and to test directly for β-carotene dioxygenase I activity an E. coli strain was constructed which is able to synthesize and accumulate β-carotene, by introducing the gene set for β-carotene biosynthesis from the bacterium Erwinia herbicola (Hundle, B. S. et al., s.a.). This approach allows the detection of retinoid formation by the fading of the colonies from yellow (β-carotene) to almost white (retinoids) and offers a fast and efficient in vitro test system to identify β-carotene dioxygenase I activity. For this purpose total RNA was isolated from Drosophila heads and cDNA was synthesized. RACE-PCR was performed with a specific oligonucleotide derived from the EST fragment and a dT₁₇-anchor-oligonucleotide. The PCR-products obtained were directly cloned into the expression vector pBAD-TOPO and transformed into the described E. coli strain. After plating the bacteria on LB-media containing 0.2% L-arabinose to induce the expression of the putative β-carotene dioxygenase I, several almost white colonies were found and subjected to further analysis (FIG. 2). Overnight cultures were grown under safety red-light to minimize isomerization and unspecific cleavage of β-carotene by photo-oxidation. β-carotene and retinoids were extracted and subjected to HPLC-analyses. The control strain transformed with the vector alone lacked the ability to cleave β-carotene and no traces of retinoids were detectable. However, bacteria expressing the Drosophila cDNA contained significant amounts of retinoids in addition to β-carotene (FIG. 3a). The retinoids were identified by retention time as well as co-chromatography with authentic standards and by their absorption spectra (FIG. 4). The dominant retinal isomer was the all-trans form, with only ca. 20% of the 13-cis isomer. Depending on the time bacteria were grown after induction, significant amounts of all-trans retinol and 13-cis retinol as well as esters of these retinol isomers could be detected. The retinoid isomers found were consistent with the isomeric composition of their β-carotene precursors which were identified by a separate HPLC-system. To confirm the formation of retinals and to improve the yield of retinoids as well as the separation of their isomers, extraction was also performed in the presence of hydroxylamine. FIG. 3b shows that this treatment leads to the formation of the all-trans and 13-cis retinal oximes with a corresponding blueshift of their absorption spectra. The analyses demonstrated that besides retinal significant amounts of retinol as well as retinyl esters were formed in E. coli (Table 1). The question arose whether E. coli is also able to form retinoic acids out of retinal. For the analyses of retinoic acid formation the cells were lysed and the extracts were analyzed on an HPLC-system using an established protocol (Thaller, C. and Eichele, G., (1987) Nature 327, 625-62814). The results revealed that under these conditions significant amounts of retinal as well as retinol could be detected but that no retinoic acids were formed in E. coli.

TABLE 1

| | E. coli⁽⁻⁾-strain | E. coli⁽⁺⁾-strain |
|---|---|---|
| all-trans retinal | n. d. | 4.7 |
| 13-cis retinal | n. d. | 1.5 |
| all-trans retinol | n. d. | 8.0 |
| 13-cis retinol | n. d. | 2.4 |
| | n. d. | 1.8 |
| Σretinoids | — | 18.4 |
| β-carotene | 56.0 | 21.4 |

n. d.: not detectable

Molar amounts (pmol/mg dry weight) of β-carotene and retinoids in the E. coli⁽⁺⁾-strain and in the E. coli⁽⁻⁾-strain from bacteria cultures which have been grown for 16 h at 28° C.

Taken together, these results demonstrate that the cloned cDNA encodes a β-carotene dioxygenase and correspondingly it was named β-diox I. Since exclusively retinoids, i.e. C₂₀ compounds, were found in the E. coli test system, it must be supposed that a centric cleavage of β-carotene is catalyzed, resulting in the formation of two molecules of retinal.

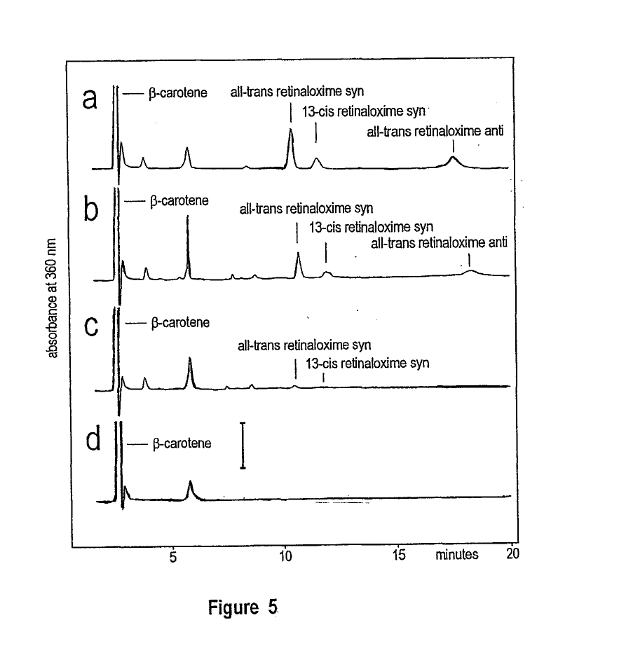

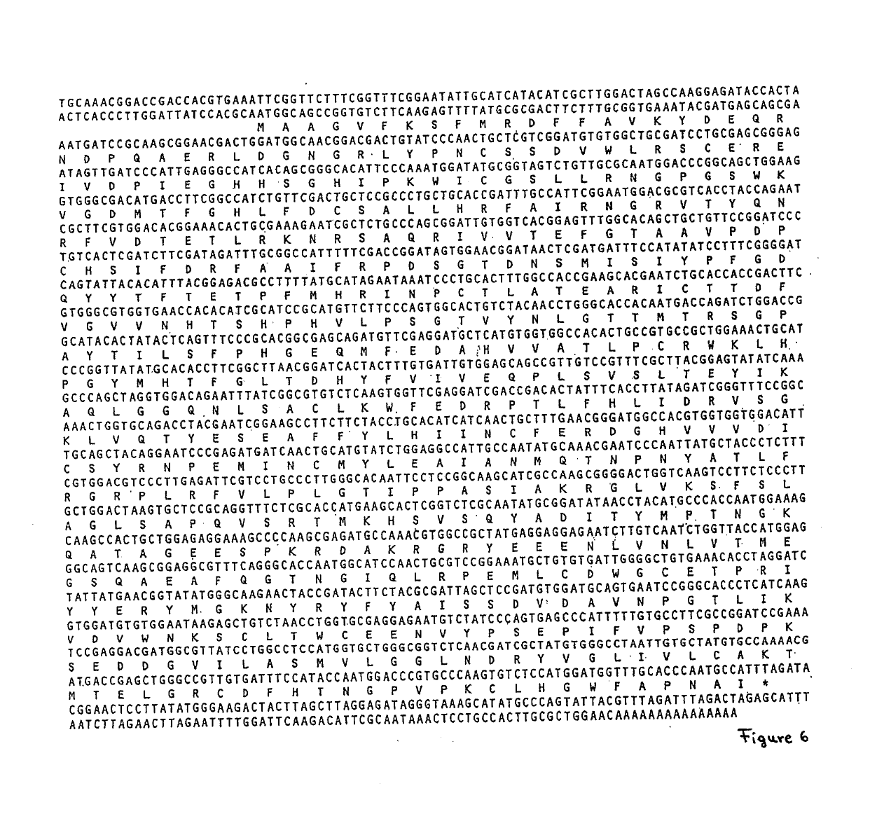

For further analysis of the enzymatic properties of β-diox I, the cDNA was cloned in the expression vector pGEX4T-1 and expressed as a fusion protein. To exclude that the N-terminal fusion to the gluthatione-S-transferase abolish the enzymatic activity, the construct (β-diox-gex) was transformed into the β-carotene synthesizing E. coli strain. Using the test described above, it could be shown that retinoids were formed to the same extent compared to the unfused β-diox I (data not shown). After expression of β-diox-gex in E. coli, the protein was subsequently purified by affinity-chromatography. The purification could be achieved without the addition of detergents indicating that the fusion-protein was soluble and not tightly associated to membranes. To test for enzymatic activity in vitro, 1 μg of the purified protein was incubated for 2 h in the presence of β-carotene in an assay containing 0.05% Triton-X-100. For the analyses of the products formed, the reaction was stopped by the addition of hydroxylamine/methanol and the products were analyzed by HPLC after extraction. The analyses revealed the formation of retinal (FIG. 5). The addition of FeSO4/ascorbate in the assays led to an increase in the formation of the cleavage product (FIG. 5A) while the conversion of β-carotene to retinal could be inhibited by the addition of EDTA (FIG. 5C). These results indicate that the enzymatic activity of the dioxygenase depend on iron as has been reported in several in vitro systems from animal origin. Taken together, the enzymatic activity of β-diox I characterized so far in the E. coli system could as well be measured in vitro with the purified protein and led to the formation of the same product.

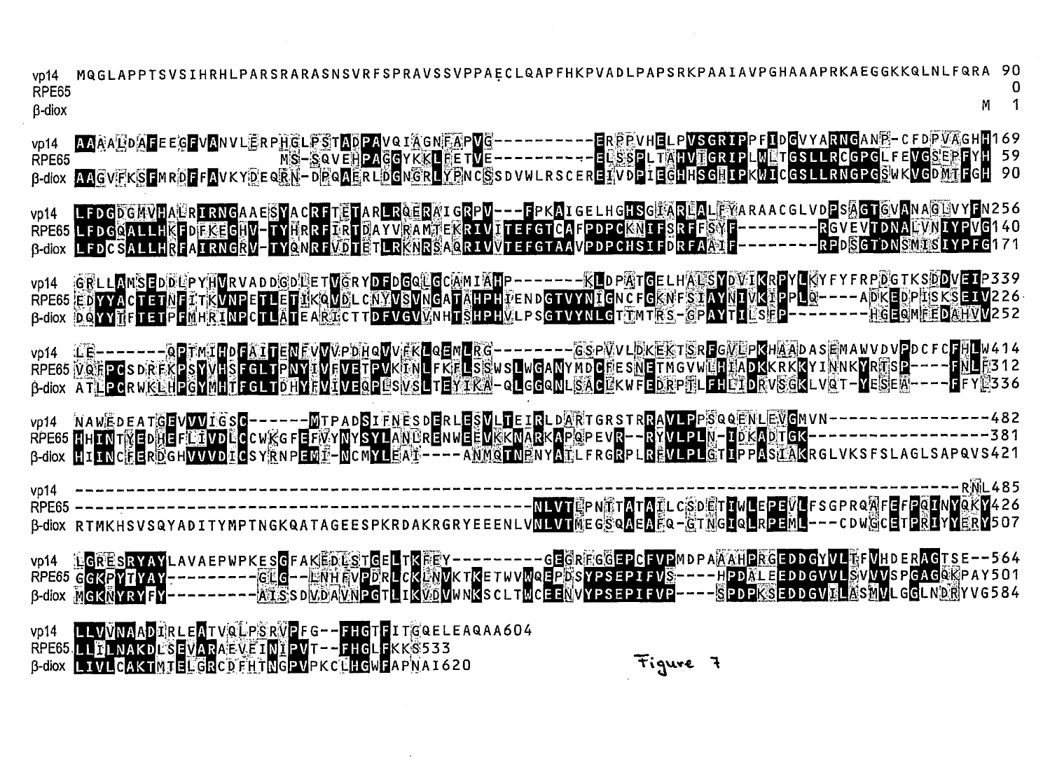

The sequence analyses revealed that the cDNA encoded a protein of 620 amino acids (SEQ ID No. 2) with a calculated molecular mass of 69.9 kDa (FIG. 6). The deduced amino acid sequence shares sequence homology to the plant carotenoid dioxygenase vp14, to lignostilbene synthase from Pseudomonas paucimobilis and to several proteins of unknown function in the Cyanobacterium Synechocystis. The highest sequence homology, however, was found to RPE65, a protein from the retinal pigment epithelium (RPE) in vertebrates, first described in bovine eyes. RPE65 and β-diox I exhibit 36.7% overall sequence identity. The alignment of the deduced amino acid sequences of β-diox I, RPE65 and vp14 performed with the program Map showed a distinct pattern of conserved regions (FIG. 7). Compared to RPE65 and vp14, the insect protein possesses a long extension close to the C-terminus. The N-terminal extension of the plant protein vp14 relative to its animal homologues is most probably due to a target sequence for plastid import The sequence homologies of β-diox II with bacterial and plant dioxygenases suggest that we are dealing with a new type of dioxygenases present in bacteria, plants and animals.

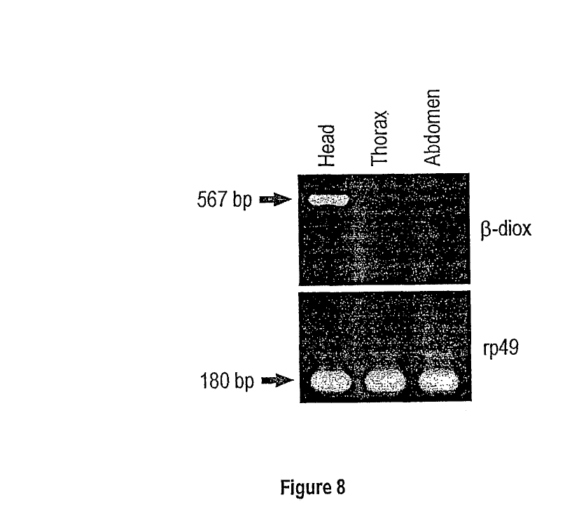

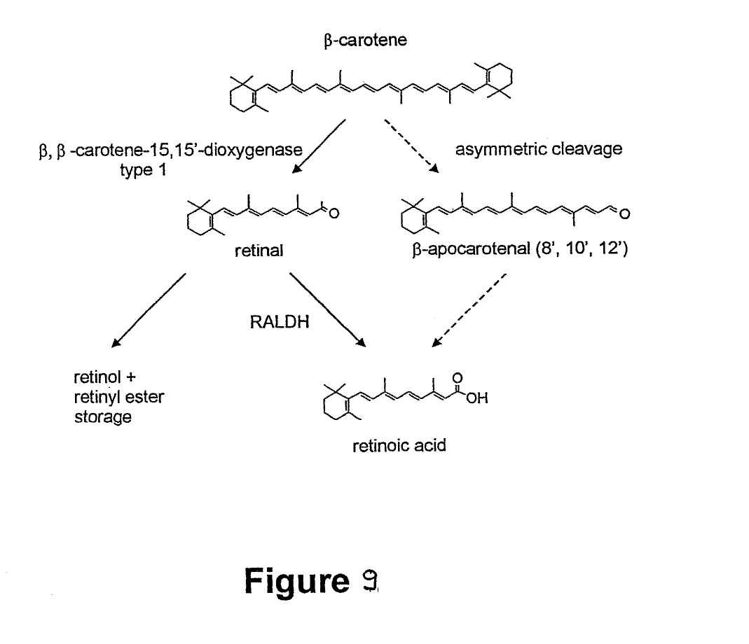

The expression pattern of β-diox I mRNA was investigated by RT-PCR As shown in FIG. 8 the mRNA was restricted exclusively to the head while in thorax and abdomen no β-diox I mRNA could be detected by this method. Although flies use 3-hydroxyretinals for vision, it has been shown that besides 3-hydroxycarotenoids (zeaxanthin and lutein) β-carotene can serve as suitable precursor. In addition, it has been demonstrated that flies are able to hydroxylate retinal at position 3 of the β-ionone ring and to form the unusual enantiomer (3S)-3-hydroxyretinal, which is the unique chromophore of cyclorrhaph flies. These results demonstrated that, in Drosophila, β-carotene cleavage and further metabolism of retinoids as well as the visual cycle are all located in the same part of the body.

Cloning of a cDNA Encoding a New Type of Carotene Dioxygenase (β-diox II)

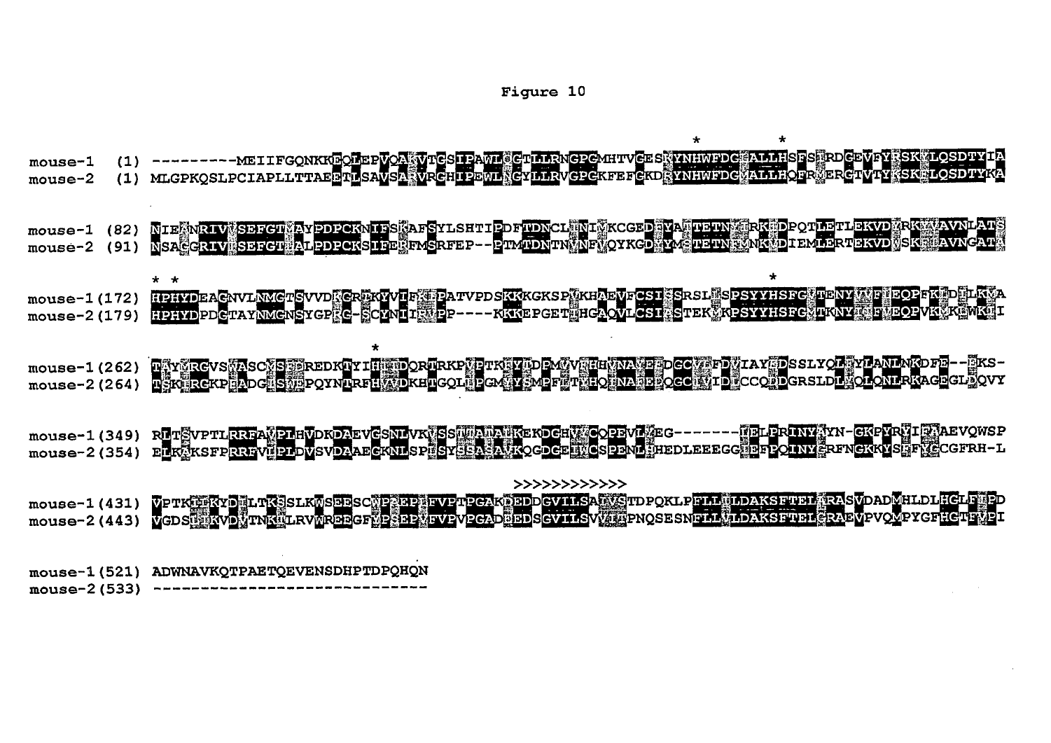

For the cloning of cDNAs encoding putative β-carotene dioxygenases, we searched mouse EST-data bases and found two EST-fragments with significant peptide sequence similarity to the so far characterized β-diox I from Drosophila. One EST-fragment (AW044715) encoded the mouse β-diox I (Redmond, T. M., Gentleman, S., Duncan, T., Yu, S., Wiggert, B., Gantt, E., and Cunningham, F. X. Jr. (2000) J. Biol Chem. online), while the other (AW611061) had significant similarity to the Drosophila, chicken, and mouse β-diox I as well as mouse RPE65. However, it was not identical and thus represented a new heretofore unknown representative of this type of dioxygenases. To obtain a full-length cDNA, we designed up-stream primers deduced from the EST-fragment. Then we performed RACE-PCR on a total RNA preparation derived from liver of a 7 week old. BALB/c male mouse. The PCR product was cloned into the vector pBAD-TOPO and sequence analyses were carried out. The cDNA (SEQ ID No. 16) encoded a protein of 532 amino acids. Sequence comparison revealed that the deduced amino acid sequence (SEQ ID No. 17) shared 39% sequence identity with the mouse β,β-carotene 15,15′-dioxygenase (β-diox I) (FIG. 10). Several highly conserved stretches of amino acids and six conserved histidines probably involved in binding the cofactor Fe²⁺ are found, indicating that the encoded proteins belong to the same type of enzymes. Thus, in mouse, besides the β-diox I and RPE65, a third type of polyene chain dioxygenase, β-diox II, exists.

The New Type of Carotene Dioxygenase Catalyzes the Asymmetric Cleavage of β-carotene Resulting in the Formation of β-10′-apo-carotenal and β-ionone

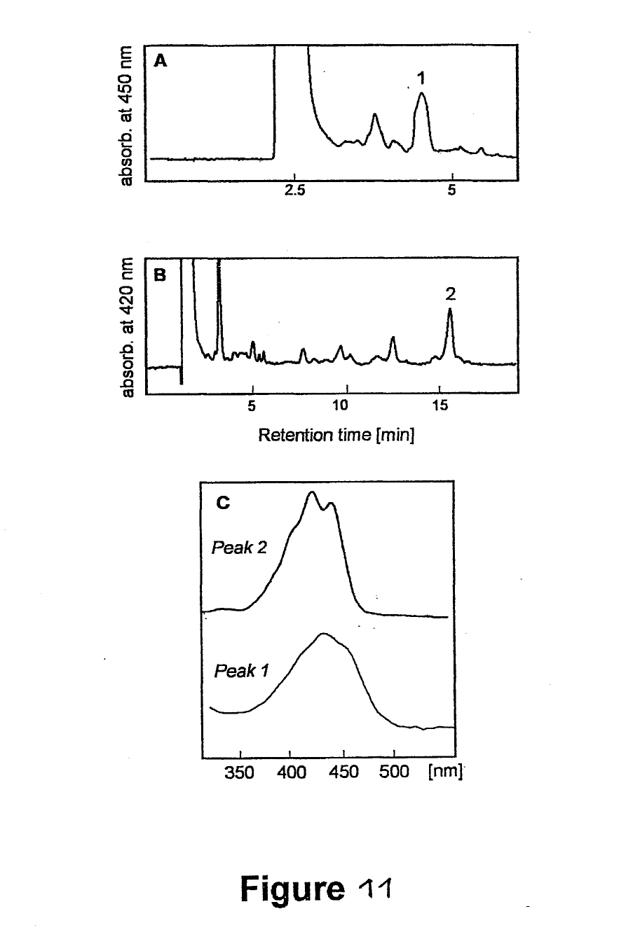

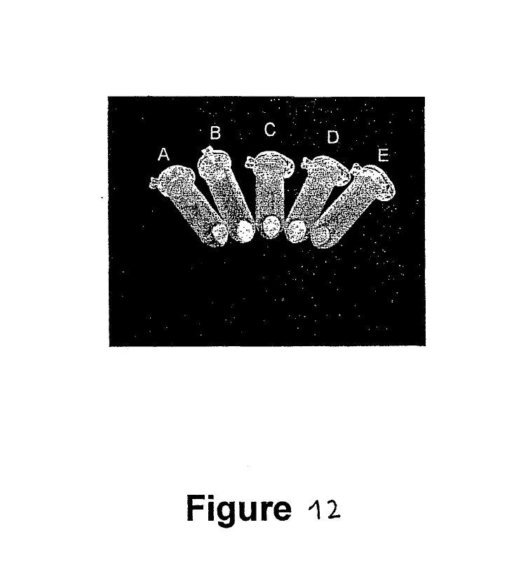

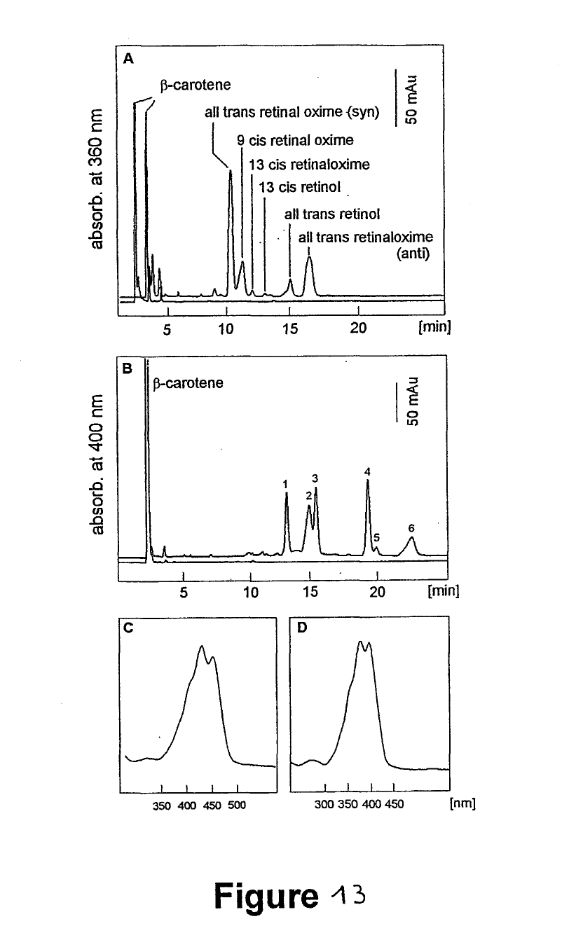

For functional characterization of β-diox II, we expressed it as a recombinant protein in E. coli and performed an in vitro test for enzymatic activity under the conditions described for β-diox I (Nagao, A., During, A., Hoshino, C., Terao, J., Olson, J. A. (1996) Arch. Biochem. Biophys. 328, 57-63). HPLC analysis revealed that no retinoids are formed from β-carotene. However, a compound with a retention of 4.6 min could be detected (FIG. 11A). In the presence of hydroxylamine during extraction, the retention time of this compound shifted from 4.6 min to 16 min, indicating that the compound has an aldehyde group from which the corresponding oxime can be formed (FIG. 11B). The increase of the putative β-carotene cleavage product catalyzed by the new type of β-carotene dioxygenase was linear up to two hours of incubation time. The UV/VIS absorption spectra of the compounds resembled those of β-apocarotenal or β-apocarotenaloxime (FIG. 11C). However, they were not identical with 8′-β-apocarotenal/oxime and 12′-β-apocarotenal/oxime, as judged by comparing the spectra of reference substances in stock in our laboratory. The UV/VIS spectra of these compounds resembled the spectra of β-10′-apocarotenal (424 nm) and β-10′-apocarotenaloxime (435 nm) as found in the literature (Barua, A. B., and Olson, J. A. (2000) J. Nutr. 130, 1996-2001). The turnover rates and, therefore, the amounts of cleavage product formed were quite low in vitro as already observed for the β-dioxs. To obtain large amounts of this substance for further chemical analysis, we decided to take advantage of an E. coli test system already successfully used to characterize the β-diox I from Drosophila. As a control we expressed the β-diox I from mouse. This test system offered the advantage to be able to visualize β-carotene cleavage by a color shift of the bacteria from yellow to almost white in the case of retinoid formation from β-carotene. While the E. coil strain expressing the β-diox I from mouse becomes white, in the E. coli strain expressing β-diox II no such pronounced color shift becomes visible, indicating that the enzyme catalyze β-apocarotenal formation in E. coli (FIG. 12). In the E. coli strains expressing β-diox II from mouse, the β-carotene content was significantly reduced (22.8 pmol/mg dry weight compared to 60.9 pmol/mg dry weight of the control strain). To identify these compounds, they were extracted and subjected to HPLC analyses as has been described above. Two classes of substances with absorption maxima at 424 nm and 386 nm, respectively, could be identified (FIGS. 13B and C). The occurrence of compounds with the same absorption spectra but different retention times could be due to the stereoisomeric composition of the products formed and/or due to the syn and anti configuration of the oximes formed. This result was already obtained upon analyzing β-diox I from the fly. Depending on the induction time, first the putative β-10′-apocarotenal and then the putative β-10′-apocarotenol becomes detectable, indicating that the aldehyde is converted to the corresponding alcohol in E. coli (data not shown). The conversion of retinal to the corresponding alcohol retinol in E. coli has been already found by expressing the β-diox I from Drosophila or from mouse as shown here (FIG. 13A). To positively identify the putative β-10′-apocarotenal formed, we converted it to the corresponding β-10′-apocarotenaloxime and subjected it to LC-MS analyses. Since the system was operated in the APcI⁺-mode, quasimolecular ions generally appear as [M+H]⁺ signals. 10′-β-apocarotenaloxime was identified by its quasimolecular ion at m/z 392 [M+H]⁺, being the base peak of the spectrum. The even-numbered [M+H]⁺ mass signal clearly proves the presence of a nitrogen in the compound and thus establishes the transformation of the aldehyde group into the corresponding oxime. Fragmentation of the polyene chain, yielding characteristic daughter ions, was not observed. Additionally, the characteristic UV spectrum, showing maxima at 405 nm (shoulder), 424 nm, and 446 nm, is in accordance with the chromophoric system of 10′-β-apocarotenaloxime and consistent with spectroscopic data reported previously (Barua, A. B., and Olson, J. A. (2000) J. Nutr. 130, 1996-2001).

Thus, from β-carotene β-10′apocarotenal is formed. However, the second compound which should result from the oxidative cleavage of β-carotene at the 9′,10′ double bond of β-carotene, β-ionone, was not detectable by HPLC. This could be either due to its volatility and/or its being partitioned to the medium. Therefore, we analyzed the bacterial growth medium after solid phase extraction of lipophilic compounds by GC-MS. In the medium of this E. coli strain, besides large amounts of indole, significant amounts of β-ionone could be detected which could be not found in the medium of the E. coli control strain. Taken together, the analyses demonstrated that β-diox II catalyzes the asymmetric cleavage of β-carotene at the 9′,10′ carbon double bond, resulting in the formation of β-10′-apocarotenal and β-ionone. Therefore, we have termed this enzyme β,β-carotene-9′,10′-dioxygenase (β-diox II). However, it should be noted that β-diox II from other sources not identified herein may alternatively attack other double bonds. Therefore, the activity of β-diox II, i.e. to cleave β-carotene asymmetrically, is not restricted to the 9′,10′ carbon double bond as disclosed above.

To test whether the enzyme catalyzes the oxidative cleavage of carotenes different from β-carotene, we transformed it into an E. coli strain able to synthesize and accumulate lycopene (FIG. 12). The experiment was performed as described above. In this strain significant amounts of putative apolycopenals become detectable. This could be shown by converting the aldehydes to the corresponding oximes (data not shown). Therefore, the new type of carotene dioxygenase catalyzes the oxidative cleavage of lycopene in the E. coli test system as well, resulting in the formation of apolycopenals being tentatively identified by their UV/VIS spectra

Cloning of cDNAs Encoding the New Type of Carotene Dioxygenase from Human and Zebrafish

To verify the existence of this second type of dioxygenase in other metazoan organisms, we searched for EST-fragments with sequence identity in the data base. We found EST-fragments from human and zebrafish. Then, we cloned and sequenced the full-length cDNAs. The cDNA (SEQ ID No. 20) cloned from total RNA derived from human liver encodes a protein of 556 amino acids (SEQ ID No. 21), while the cDNA (SEQ ID No. 18) isolated from zebrafish encodes a protein of 549 amino acids (SEQ ID No. 19). The deduced amino acid sequences share 72 and 49% sequence identity to the mouse β-diox II. We performed phylogenetic tree calculation based on a sequence distance method and utilizes neighbor joining algorithm with the deduced amino acid sequences of the metazoan polyene chain dioxygenases and the plant VP14. As shown in FIG. 15, in vertebrates three groups of polyene chain dioxygenases are found—the two different β-carotene dioxygenases (I and II) and RPE65. In Drosophila and Caenorhabditis elegans, only one type of dioxygenase (I) was found in the entire genome. As judged by the E. coli test system, the C. elegans dioxygenase catalyzes the symmetric clevage of β-carotene to form retinal. The sequence analysis revealed that the three vertebrate polyene chain dioxygenases emerged most probably from a common ancestor. Therefore, the occurrence of additional genes encoding this type of enzymes, the β-diox and the RPE65, is apparently related to vertebrate carotene/retinoid metabolism.

Tissue Specific Expression of the New Type of Carotene Dioxygenase

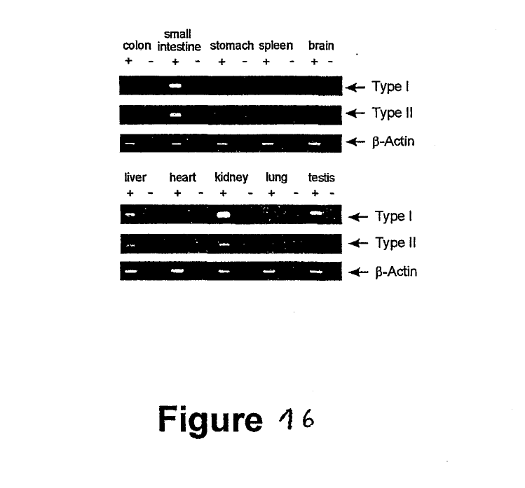

We analyzed total RNA from several tissues of 7 week old BALB/c mice (male and female) and estimated the steady-state mRNA levels of the two types of carotene dioxygenases by RT-PCR analyses. RT-PCR products of both types of carotene dioxygenase mRNAs became detectable in small intestine, liver, kidney and testis. The mRNA for the new type of carotene dioxygenase was additionally present in spleen and brain, while low abundance steady-state mRNA levels for both types of carotene dioxygenases were detectable in lung and heart (FIG. 16). The intactness of the RNA preparations was verified by analyzing the β-actin mRNA. By omitting the reverse transcriptase in the assays, it could be shown that the RT-PCR products derived from mRNA and not from DNA contaminations. By using a multiple tissue mRNA blot, analyzed with riboprobe of the human cDNA, we could find a 2.2 kb message in heart and liver for the new type of carotene dioxygenase while a transcript of 2.4 kb for the β-diox II was found mainly in kidney (data not shown).

Discussion Chronically Reflecting the Above Results

According to the invention Drosophila β-diox I has been the first β-carotene dioxygenase to be molecularly identified. In the course of the experiments leading to the principles of the present invention it could be proven that there are two alternative pathways starting from β-carotene as substrate being characterized by the different enzymatic activities of the homologous β-diox I and II gene types. The information disclosed herein provides the key to opening up a broad field for further investigation of carotenoid/retinoid metabolism in animals.

The β-diox I encodes a protein of 620 amino acids with a calculated molecular mass of 69.9 kDa. The sequence comparison revealed that β-diox I belongs to a new type of dioxygenases so far found only in bacteria and plants. Enzymatic activity of β-diox I could be measured under the same condition as has been reported for the plant carotenoid cleavage enzyme vp14 responsible for the cleavage of 9-cis-neoxhantin in the ABA biosynthetic pathway. In animals, it has been reported that β-carotene dioxygenase activity depends on iron. The addition of FeSO₄/ascorbat to the assay led to an increase of the enzymatic activity while the addition of EDTA decreased the formation of retinal significantly. Enzymatic activity could be measured without the addition of cofactors such as thiol reagents or electron acceptors. This indicates that β-diox depends on Fe²⁺ and that no other cofactors are required for enzymatic activity just as reported for the plant vp14. Since β-carotene is not soluble in an aqueous environment, tests for enzymatic activity were carried out in the presence of 0.05% Triton-X-100. In vivo β-carotene is not freely diffusible and must be associated with lipophilic structures such as membranes or binding proteins. Therefore, the question arose whether β-diox is bound to membranes to interact with its lipophilic substrate. The β-diox-fusion protein could be purified without the addition of detergents and this points to its soluble state rather than to its membrane bound topology. However, the glutathione-S-transferase part of the fusion protein may also contribute to its solubility. Since the visual chromophore of Drosophila is 3-hydroxy-retinal, we tested whether β-diox I was able to use zeaxhantin as a substrate to form directly this hydroxylated retinoid but under the conditions we applied the enzyme failed to catalyze this reaction. In addition, we expressed β-diox I in a zeaxhantin accumulating E. coli strain but only the formation of non-hydroxylated retinoids could be detected. In this E. coli strain significant amounts of β-carotene, the direct precursor of zeaxhantin, were found which can serve as a substrate for β-diox I. An explanation may be in the fact that Drosophila is able to hydroxylate retinal at position 3 of the β-ionone ring. Taken together, we could show that β-diox I catalyzes the symmetric cleavage of β-carotene.

The β-diox I gene is located at position 87F on chromosome 3 in the Drosophila genome. Precisely in this region a Drosophila mutant, ninaB, has been mapped by cytological methods (FlyBase Map section 87). The mutant phenotype has a reduced rhodopsin content in all photoreceptor classes. However, the mutant phenotype can be rescued by the dietary supplement of retinal but not by even high doses of β-carotene. Both, the availability of the visual pigment chromophores as well as the transcriptional regulation by retinoic acid of the protein moiety (opsin) of the visual pigment depend on β-diox enzymatic activity. Thus, it could be proven that the ninaB phenotype is caused by a mutation in β-diox I.

The highest sequence homology of β-diox I is found to RPE65, a protein first described in bovine eyes. Therefore the question arises whether RPE65 is the vertebrate equivalent to β-diox I. Although the exact function of RPE65 is not yet known, a role in vitamin A metabolism has been proposed, and recently, it was found that mutations in the gene are responsible for a severe form of early onset retinal dystrophy in humans. In the eyes of mice where the RPE65 gene has been disrupted, all-trans vitamin A accumulates. Therefore, it has been concluded that RPE65 takes part in the isomerization of all-trans to 11-cis vitamin A in the mammalian visual cycle. However, after removal of RPE65 from RPE-membrane fractions the isomerization of all-trans-retinol into 11-cis-retinol remained unaffected. To our knowledge a β-carotene dioxygenase activity has never been reported in the RPE nor have significant amounts of its substrate β-carotene been measured in vertebrate eyes. We expressed RPE65 cloned by RT-PCR from the bovine RPE in the test system described but neither the formation of retinoids nor the formation of eccentric cleavage products such as apocarotenals could be detected. Therefore, the exact function of RPE65 remains to be further investigated, and we propose that other, as yet undiscovered, members of this family with different tissue specificity (small intestine, liver) are responsible for the vertebrate β-carotene dioxygenase activity. The sequence homology of β-diox I with RPE65, as well as with plant and bacterial dioxygenases, suggests that we are dealing with a new type of dioxygenases catalyzing the cleavage of a conjugated carbon double bond. This reaction type is involved in the cleavage of carotenoids as well as in a variety of other compounds. The described E. coli test system provides a powerful tool to characterize new genes involved in retinoid formation and to screen for potential agonists or antagonists of the enzymes according to the invention. Furthermore, the retinoid producing E. coli strain was successfully used to identify further steps in carotene/retinoid metabolism.

According to a further aspect of the present invention we report on the cloning, characterization, and tissue specific expression of a second new type of carotene dioxygenase from mouse, human and zebrafish catalyzing the asymmetric cleavage of β-carotene. By expressing the enzyme in a β-carotene synthesizing E. coli strain, β-apocarotenal formation at the expense of β-carotene was shown. The cleavage products formed could be identified by their absorption spectra, by the conversion of the aldehyde to the corresponding oxime and by LC-MS or GC-MS as being β-10′-apocarotenal and β-ionone. In vitro, the enzyme catalyzed the same reaction as in the E. coli test system. Thus, the characterized enzyme catalyzed the oxidative cleavage at the 9′-10′ double bond in the polyene backbone of its substrate β-carotene.

Besides the overall sequence identity to the β-diox I discussed hereinabove, there is a distinct conserved pattern of histidine residues; which can be involved in the binding of the cofactor Fe²⁺. Thus, including RPE65, three different representatives of the polyene chain dioxygenase family are found in vertebrates. While the biochemical function of the RPE65 protein remains to be elucidated, we show that besides symmetrical cleavage of β-carotene asymmetric cleavage also occurs, resolving the controversial debate on the significance of this reaction positively. The analysis of the tissue specific expression showed that mRNAs for both enzymes are found together in several tissues, e.g. small intestine and liver. These findings verify biochemical results on the molecular level that both symmetric and asymmetric cleavage of β-carotene can be found in the same tissue. The expression patterns in mouse and human were not consistent. This could be either due to interspecies differences in carotene metabolism or reflect differences in the age and nutritional status of the individuals investigated, thus possibly presenting an additional factor to explain the conflicting results obtained in several investigations. In earlier studies conducted with tissue homogenates a variety of β-apocarotenals of different chain length resulting from asymmetric β-carotene cleavage could be found. Therefore, the term random cleavage was used for this reaction by several authors. Here we show that the enzyme β-diox II does not catalyze such side reactions instead being specific for the 9′,10′ double bond. The formation of β-apocarotenals different from 10′-β-apocarotenal found in vitro may be caused by further metabolism of the primary cleavage product or by additional yet unknown carotene dioxygenases. However, the in vitro activity of the metazoan polyene chain dioxygenases is difficult to obtain and β-apocarotenal formation from β-carotene by non-enzymatic degradation has been reported in an aqueous environment (Henry, L. K., Puspitasari-Nienaber, N. L., Jaren-Galan, M., van Breemen, R. B., Catignani, G. L., and Schwartz, S. J. (2000) J. Agric. Food Chem. 48, 5008-5013).

After the molecular identification of a cDNA encoding this new type of carotene dioxygenase (β-diox II), the question arose as to the physiological relevance in vertebrate carotene metabolism. It has been shown in rats and chicken that β-apocarotenals can be bioactive precursors for RA formation. After absorption of these compounds, first the corresponding acid is formed, then being shortened to yield retinoic acid. The same study also showed that only small proportions of β-apocarotenals are attacked by the β-diox to give retinal. This possibility could be of importance considering the co-expression of both dioxygenases in several tissues as shown here. It has further been found that several tissues are able to synthesize RA and that retinal, the primary product of the symmetric cleavage of β-carotene, was not found to be an intermediate. By analyzing RA formation from β-apocarotenals a mechanism similar to β-oxidation of fatty acids was proposed. In these studies, RA formation from β-apocarotenals was ensured by giving citral, a potent-inhibitor of retinalaldehyde dehydrogenases catalyzing the oxidation of retinal to RA. Therefore, the asymmetric cleavage reaction most likely represents the first step in an alternative pathway in the formation of RA and may contribute to RA homeostasis either of the body, certain tissues, or cells. The second product resulting from asymmetric cleavage β-ionone is known as a scent compound in plants. This short chain compound is volatile, and a putative physiological role in animals remains to be investigated.

In Drosophila vitamin A is exclusively formed by the symmetric cleavage reaction. In vertebrates the two different carotene dioxygenases β-diox I and β-diox II as well as RPE65 protein are found. Sequence comparison indicated that the vertebrate dioxygenases arose from a common ancestor. In contrast to Drosophila, in vertebrates RA plays an important role in development and cell differentiation. Thus, the existence of different β-carotene dioxygenases could be related to the emergence of RA effects. By in situ hybridization in zebrafish embryos, high steady state mRNA levels of the zebrafish homologue of the β-diox were found before gastrulation. The zebrafish homologue to the β-carotene-9′,10′-dioxygenase could only be detected after organogenesis. The finding of high steady state mRNA levels of the β-diox I at early times in development has been reported for mouse (Redmond, T. M., Gentleman, S., Duncan, T., Yu, S., Wiggert, B., Gantt, E., and Cunningham, F. X. Jr. (2000) J. Biol Chem. Online). This indicates that retinoid formation from β-carotene catalyzed by the symmetric oxidative cleavage reaction may contribute to the retinoid homeostasis of the embryo. Therefore, besides maternal preformed vitamin A de novo biosynthesis from the provitamin seems to be an important source for retinoids during development. However, the asymmetric cleavage reaction may contribute to RA formation in certain tissues during later stages of development. In this context, the expression of the β-diox II in brain and lung could be of relevance. In cell differentiation processes in the nervous system, RA plays an important role. In a ferret model, under certain conditions such as exposure to cigarette smoke β-carotene toxicity on lung has been reported. In this context asymmetric cleavage of β-carotene was discussed to be involved in these toxic effects (for review, Russell, R. M. (2000) Am. J. Clin. Nutr. 71, 878-884). Furthermore, RA formation from β-carotene has been found in vitro in the testis, small intestine, liver, kidney and lung. Here, we show that in all these tissues mRNA encoding the two different types of carotene dioxygenases are found. This indicates that besides small intestine and liver, several tissues may contribute to their own RA homeostasis by endogenous retinoid formation from β-carotene, until now an underestimated, unappreciated feature in retinoid homeostasis.

As judged in an E. coli test system, the enzyme was also able to catalyze the oxidative cleavage of lycopene. This indicates with respect to substrate specificity that the polyene chain backbone of carotenes plays an important role while the ionone ring structures of β-carotene seem to be of marginal relevance. This result was also obtained upon analyzing the mouse β-diox I. Favorable effects of lycopene on human health have been reported. Lycopene is accumulated primarily in liver but also in intestine, prostate and testis, tissues in which both β-diox I and β-diox II mRNAs are expressed. The cleavage of lycopene and the formation of apolycopenals are indicative of a putative role in vertebrate physiology. In vertebrates, several nuclear receptors with unknown ligands exist, e.g. orphan receptors. Besides being a putative precursor for RA formation in the case of β-carotene cleavage, it may be speculated that the compounds formed by the asymmetric cleavage reaction of β-carotene and/or lycopene could represent putative ligands for these receptors.

Taken together, the data presented here led to the molecular identification of an enzyme, β-carotene-9′,10′-dioxgenase, catalyzing the asymmetric cleavage of β-carotene. Thus, besides the symmetric cleavage of β-carotene a second enzymatic activity is present in vertebrates. The molecular identification of enzymes involved in the cleavage of β-carotene will open new avenues of research on the impact of metabolites derived from carotenes in animal physiology and human health.

In recent years there has been a tremendous increase in the understanding of retinoid receptors and their ligands, as well as their diverse roles in development and cell differentiation. With the present findings, the impact of the cleavage reaction on tissue distributions, the isomeric specificity of retinoids and the regulation of the vitamin A uptake may soon be further elucidated.

Furthermore, the identification of the cDNAs encoding the β-carotene dioxygenases I and II has a tremendous impact for medicine, pharmacological and biotechnological applications. In medicine, the cloning of the corresponding gene from humans or mammals allows the physiological characterization of mammal carotene/retinoid metabolism in more detail and will have impact of the multitude of effects caused by vitamin A and its derivatives and will therefore offer several therapeutical applications.

It is known that vitamin A deficiency is a serious problem. The cDNA equipped with the necessary regulatory sequences can be used for expressing it into retinoid free organisms such as most plants, most bacteria, and fungi. Therefore, vitamin A production in crops and in microorganisms used in food-technology or spoken more generally vitamin A production in as yet retinoid-free organism which are able to synthesize provitamin A (β-carotene) can be achieved according to the present invention.

Obviously, many modifications and variations of the present invention are possible in the light of the above teachings. It is, therefore, to be understood that within the scope of the appended claims, the invention may be practised otherwise than as specifically described.

[image]

1. An isolated β-carotene dioxygenase (β-diox II) polypeptide or functional fragment thereof having the biological activity of specifically cleaving β-carotene and lycopene to form β-apocarotenal and β-ionone, and apolycopenals, respectively.

2. The β-diox II polypeptide or functional fragment thereof according to claim 1 comprising one or more of the amino acid sequences selected from the group consisting of amino acid sequences extending from 39 to 47, 96 to 118, 361 to 368, and 466 to 487 of SEQ ID No. 17, from 55 to 63, 112 to 134, 378 to 385, and 482 to 503 of SEQ ID No. 19, and from 59 to 67, 116 to 138, 385 to 392, and 490 to 511 of SEQ ID No. 21.

3. The β-diox II polypeptide or functional fragment thereof according to claim 1 or 2 having an amino acid sequence which is at least 45% identical to the amino acid sequence as set out in SEQ ID Nos. 17, 19, or 21.

4. The β-diox II polypeptide or functional fragment thereof according to claim 1 or 2 having an amino acid sequence which is at least 60% identical to the amino acid sequence as set out in SEQ ID Nos. 17, 19, or 21.

5. The β-diox II polypeptide or functional fragment thereof according to claim 1 or 2 having an amino acid sequence which is at least 75% identical to the amino acid sequence as set out in SEQ ID Nos. 17, 19, or 21.

6. The β-diox II polypeptide or functional fragment thereof according to claim 1 or 2 having an amino acid sequence which is at least 90% identical to the amino acid sequence as set out in SEQ ID Nos. 17, 19, or 21.

7. The β-diox II polypeptide or functional fragment thereof according to claim 1 or 2 having the amino acid sequence as set out in SEQ ID Nos. 17, 19, or 21, or parts thereof.

8. The β-diox II polypeptide or functional fragment thereof according to claim 1 or 2 having an amino acid sequence as encoded by a DNA sequence selected from the group consisting of:
(a) the DNA sequence as set out in either SEQ ID No. 16 and/or SEQ ID No. 18 and/or SEQ ID No. 20, and complementary strands thereof; and
(b) the DNA sequences extending from position 115 to 141, 286 to 354, 1081 to 1104, and 1396 to 1461 of SEQ ID No. 16, or complementary strands thereof; and
(c) the DNA sequences extending from position 191 to 217, 362 to 430, 1160 to 1183, and 1472 to 1537 of SEQ ID No. 18, or complementary strands thereof; and
(d) the DNA sequences extending from position 175 to 201, 346 to 414, 1153 to 1176, and 1468 to 1533 of SEQ ID No. 20, or complementary strands thereof; and
(e) DNA sequences which hybridize under high-stringency conditions to the DNA sequences or complementary strands as defined in (a), (b) (c) and (d) or functional fragments thereof; and
(f) DNA sequences which would hybridize to the DNA sequences as defined in (a), (b), (c), (d) and (e) but for the degeneracy of the genetic code.

9. A DNA molecule comprising a DNA sequence encoding a β-diox II polypeptide or functional fragment thereof according to any of claims 1 through 8.

10. A DNA molecule comprising a DNA sequence for use in securing expression of a β-diox II polypeptide or functional fragment thereof having the biological activity of specifically cleaving β-carotene and lycopene to form β-apocarotenal and β-ionone, and apolycopenals, respectively, or for use in the determination of the presence of nucleic acid(s) being characteristic for said polypeptide or functional fragment thereof, which is selected from the group consisting of:
(a) the DNA sequence as set out in either SEQ ID No. 16 and/or SEQ ID No. 18 and/or SEQ ID No. 20, and complementary strands thereof, and
(b) the DNA sequences extending from position 115 to 141, 286 to 354, 1081 to 1104, and 1396 to 1461 of SEQ ID No. 16, or complementary strands thereof; and
(c) the DNA sequences extending from position 191 to 217, 362 to 430, 1160 to 1183, and 1472 to 1537 of SEQ ID No. 18, or complementary strands thereof; and
(d) the DNA sequences extending from position 175 to 201, 346 to 414, 1153 to 1176, and 1468 to 1533 of SEQ ID No. 20, or complementary strands thereof; and
(e) DNA sequences which hybridize under high-stringency conditions to the DNA sequences or complementary strands as defined in (a), (b) (c) and (d) or functional fragments thereof; and
(f) DNA sequences which would hybridize to the DNA sequences as defined in (a), (b), (c), (d) and (e) but for the degeneracy of the genetic code.

11. The DNA molecule according to claim 9 or 10 comprising a DNA sequence which is a cDNA, genomic or manufactured DNA sequence.

12. The DNA molecule according to any of claims 9 to 11, further comprising at least one selectable marker gene or cDNA operably linked to a constitutive, inducible or tissue-specific promoter sequence allowing its expression in bacteria, fungi including yeast, insect, animal or plant cells, seeds, tissues or whole organisms.

13. The DNA molecule according to any of claims 9 to 12, wherein the coding nucleotide sequence is fused with a suitable plastid transit peptide encoding sequence, both of which preferably are expressed under the control of a tissue-specific or constitutive promoter.

14. A plasmid or vector system comprising one or more DNA molecules according to any of claims 9 to 13.

15. A process for producing a β-diox II polypeptide comprising the steps of:
(a) expressing a polypeptide encoded by a DNA according to any of claims 9 to 14 in a suitable host, and
(b) isolating said β-diox II polypeptide.

16. A protein product obtained by the process of claim 15.

17. A procaryotic or eucaryotic host cell, seed, tissue or whole organism transformed or transfected with the DNA molecule according to any of claims 9 to 13 or with the plasmid or vector system according to claim 14 in a manner enabling said host cell, seed, tissue or whole organism to express a polypeptide or functional fragment thereof having the biological activity of specifically cleaving β-carotene and lycopene to form β-apocarotenal and β-ionone, and apolycopenals, respectively, and/or having the capability of specifically binding to antibodies raised against said polypeptide or functional fragment thereof.

18. The procaryotic or eucaryotic host cell, seed, tissue or whole organism according to claim 17 selected from the group consisting of bacteria, fungi including yeast, insect, animal and plant cells, seeds, tissues or whole organisms.

19. The procaryotic host cell or whole organism according to claim 18 being a bacterium selected from the group consisting of proteobacteria including members of the alpha, beta, gamma, delta and epsilon subdivision, gram-positive bacteria including Actinomycetes, Firmicutes, Clostridium and relatives, flavobacteria, cyanobacteria, green sulfur bacteria, green non-sulfur bacteria, and archaea.

20. The procaryotic host cell or whole organism according to claim 19 belonging to the group of proteobacteria selected from the group consisting of Agrobacterium, Rhodobacter, ammonia-oxidizing bacteria such as Nitrosomonas, Enterobacteriaceae, Myxobacteria such as Myxococcus, with Agrobacterium aureus, Rhodobacter capsulatus, Nitrosomonas sp. ENI-11, Escherichia coli and Myxococcus xanthus being preferred.

21. The procaryotic host cell or whole organism according to claim 19 belonging to the group of gram-positive bacteria selected from the group consisting of Actinomycetes and Firmicutes including Clostridium and relatives such as Bacillus and Lactococcus, with Bacillus subtilis and Lactococcus lactis being preferred.

22. The procaryotic host cell or whole organism according to claim 19 belonging to the group of flavobacteria selected from the group consisting of Bacteroides, Cytophaga and Flavobacterium, with Flavobacterium such as Flavobacterium ATCC21588 being preferred.

23. The procaryotic host cell or whole organism according to claim 19 belonging to the group of cyanobacteria selected from the group consisting of Chlorococcales including Synechocystis and Synechococcus, with Synechocystis sp. and Synechococcus sp. PS717 being preferred.

24. The procaryotic host cell or whole organism according to claim 19 belonging to the groups of green sulfur bacteria or green non-sulfur bacteria selected from Chlorobium or Chloroflexaceae such as Chloroflexus, respectively, with Chlorobium limicola f. thiosulfatophilum and Chloroflexus aurantiacus, respectively, being preferred.

25. The procaryotic host cell or whole organism according to claim 19 belonging to the group of archaea selected from Halobacteriaceae such as Halobacterium, with Halobacterium salinarum being preferred.

26. The eucaryotic host cell or whole organism according to claim 18 being fungi including yeast selected from the group consisting of Ascomycota including Saccharomycetes such as Pichia and Saccharomyces, and anamorphic Ascomycota including Aspergillus, with Saccharomyces cerevisiae and Aspergillus niger being preferred.

27. The eucaryotic host cell according to claim 18 being an insect cell selected from the group consisting of SF9, SF21, Trychplusiani and MB21.

28. The eucaryotic host cell according to claim 18 being an animal cell selected from the group consisting of Baby Hamster Kidney (BHK) cells, Chinese Hamster Ovarian (CHO) cells, Human Embryonic Kidney (HEK) cells and COS cells, with NIH 3T3 and 293 being most preferred.

29. The eucaryotic host cell, seed, tissue or whole organism according to claim 18 being a plant cell, seed, tissue or whole organism selected from the group consisting of eukaryotic alga, embryophytes comprising Bryophyta, Pteridophyta and Spermatophyta such as Gymnospermae and Angiospermae, the latter including Magnoliopsida, Rosopsida, and Liliopsida (“monocots”).

30. The eucaryotic host cell, seed, tissue or whole organism according to claim 29 selected from the group consisting of grain seeds, with rice, wheat, barley, oats, amaranth, flax, triticale, rye, and corn being preferred; oil seeds, with Brassica seeds, cotton seeds, soybean, safflower, sunflower, coconut, and palm being preferred; other edible seeds or seeds with edible parts selected from the group consisting of pumpkin, squash, sesame, poppy, grape, mung beans, peanut, peas, beans, radish, alfalfa, cocoa, coffee, hemp; tree nuts, with walnuts, almonds, pecans, and chick-peas being preferred; potatoes, carrots, sweet potatoes, sugar beets, tomato, pepper, cassava, willows, oaks, elm, maples, apples and bananas.

31. A method of transforming bacteria, yeast, fungi, insect, animal or plant cells, seeds, tissues or whole organisms in order to yield transformants capable of expressing a β-carotene dioxygenase (β-diox II) polypeptide or functional fragment thereof having the biological activity of specifically cleaving β-carotene and lycopene to form β-apocarotenal and β-ionone, and apolycopenals, respectively, and/or having the capability of specifically binding to antibodies raised against said polypeptide or functional fragment thereof, comprising the transformation of said bacteria, fungi including yeast, insect, animal or plant cells, seeds, tissues or whole organisms with a DNA molecule according to any of claims 9 to 13, or with a plasmid or vector system according to claim 14.

32. A transformed bacteria, fungi including yeast, insect, animal or plant cell, seed, tissue or whole organism represented by or regenerated from transformants yielded according to claim 31.

33. The transformed plant cell, seed, tissue or whole organism according to claim 32 selected from the group consisting of eukaryotic alga, embryophytes comprising Bryophyta, Pteridophyta and Spermatophyta such as Gymnospermae and Angiospermae, the latter including Magnoliopsida, Rosopsida, and Liliopsida (“monocots”).

34. The transformed plant cell, seed, tissue or whole organism according to claim 33, selected from the group consisting of grain seeds, with rice, wheat, barley, oats, amaranth, flax, triticale, rye, and corn being preferred; oil seeds, with Brassica seeds, cotton seeds, soybean, safflower, sunflower, coconut, and palm being preferred; other edible seeds or seeds with edible parts selected from the group consisting of pumpkin, squash, sesame, poppy, grape, mung beans, peanut, peas, beans, radish, alfalfa, cocoa, coffee, hemp; tree nuts, with walnuts, almonds, pecans, and chick-peas being preferred; potatoes, carrots, sweet potatoes, sugar beets, tomato, pepper, cassava, willows, oaks, elm, maples, apples and bananas.

35. An antibody specifically immunoreactive with a polypeptide of any claims 1 through 8 and 16.

36. Use of a DNA molecule according to any of claims 9 through 13 for diagnostic and/or therapeutic purposes.

37. Use of a polypeptide according to any of claims 1 through 8 and 16 for therapeutic purposes.

38. Use of an antibody according to claim 35 for the isolation and/or quantification of a polypeptide of any claims 1 through 8 and 16, and for diagnostic and/or therapeutic purposes.
